SEQUENCE LISTING (169 sequences)

SEQ ID NO: 1 - protein, 160 aa, Haemophilus influenzae
MKKIILTLSLGLLTACSAQIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 2 - protein, 160 aa, Haemophilus influenzae
MKKIILTLSLGLLTACSAQIQKAKQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 3 - protein, 160 aa, Haemophilus influenzae
MKKIILTLSLGLLTACSAQIQKAEQNDVKLAPPTDVRSGYIRLVKNVNYYIDSESIWVDNQEPQIVHFDAVVNLDRGLYVYPEPKRYARSVRQYKILNCANYHLTQIRTDFYDEFWGQGLRAAPKKQKKHTLSLTPDTTLYNAAQIICANYGKAFSVDKK

SEQ ID NO: 4 - protein, 160 aa, Haemophilus influenzae; identical to SEQ ID NO: 2

SEQ ID NO: 5 - protein, 160 aa, Haemophilus influenzae
MKKIILTLSLGLLTACSAQIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGKAFSVDKK

SEQ ID NO: 6 - protein, 160 aa, Haemophilus influenzae; identical to SEQ ID NO: 5

SEQ ID NO: 7 - protein, 160 aa, Haemophilus influenzae
MKKIILTLSLGLLTACSAQIQKAEQNDMKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGKAFSVDKK

SEQ ID NO: 8 - protein, 160 aa, Haemophilus influenzae; identical to SEQ ID NO: 2

SEQ ID NO: 9 - protein, 159 aa, Haemophilus influenzae
MKKIILTLSLGLLTACSAQIQKAEQNDMKLAPPTDVRSGYIRLVKNVNYYIDSESIVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGKAFSVDKK

SEQ ID NO: 10 - protein, 160 aa, Haemophilus influenzae
MKKIILTLSLGLLTACSAQIQKAEQNDVKLAPPTDVRSGYIRLVKNVNYYIDSESIWVDNQEPQIVHFDTVVNLDKGLYVYPEPKRYARSVRQYKILNCANYHLTQVRTDFYDEFWGQGLRAAPKKQKKHTLSLTPDTTLYNAAQIICANYGKAFSVDKK

SEQ ID NO: 11 - protein, 160 aa, Haemophilus influenzae; identical to SEQ ID NO: 5

SEQ ID NO: 12 - protein, 194 aa, Haemophilus influenzae
MKKIILTLSLGLLTACSAQTQKAEQNDVKLTPPTDVQSGYVRLVKNVNYYIDSESIWVDNQEPQIVHFDAVVNLDKGLYVYPEPKRYARSVRQYKILNCANYHLTQVRIDFYDEFWGQGLRAAPKKQKKHTLSLTPDTTLYNAAQIICANYGKAFSVDKNKKICTLISLNFIQLLGCREYSIFLQLLLFYCWHF

SEQ ID NO: 13 - protein, 195 aa, Haemophilus influenzae
MKKIILTLSLGLLTACSAQIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGKAFSVDKKIKKICTLISLNFIQLLGCREYSIFLQLLLFYCWHF

SEQ ID NO: 14 - protein, 160 aa, Haemophilus influenzae; identical to SEQ ID NO: 7

SEQ ID NO: 15 - protein, 160 aa, Haemophilus influenzae; identical to SEQ ID NO: 5

SEQ ID NO: 16 - protein, 160 aa, Haemophilus influenzae; identical to SEQ ID NO: 2

SEQ ID NO: 17 - protein, 160 aa, Haemophilus influenzae
MKKIILTLSLGLLTACSAQTQKVEQNDVKLTAPTDVRSGFVRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGKAFLVDKK

SEQ ID NO: 18 - protein, 160 aa, Haemophilus influenzae; identical to SEQ ID NO: 2

SEQ ID NO: 19 - protein, 160 aa, Haemophilus influenzae
MKKIILTLSLGLLTACSAQIQKAEQNDVKLAPPTDVRSGYIRLVKNVNYYIDSESIWVDNQEPQIVHFDAVVNLDKGLYVYPEPKRYARSVRQYKILNCANYHLTQIRTDFYDEFWGQGLRAAPKKQKKHTLSLTPDTTLYNAAQIICANYGKAFSVDKK

SEQ ID NO: 20 - protein, 160 aa, Haemophilus influenzae; identical to SEQ ID NO: 2

SEQ ID NO: 21 - protein, 160 aa, Artificial Sequence
  902550Z19
MKKIILTLSLGLLTACSAQTQKVEQNDVKLTPPTDVRSGYVRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGKAFSVDKK

SEQ ID NO: 22 - protein, 160 aa, Haemophilus influenzae; identical to SEQ ID NO: 2

SEQ ID NO: 23 - protein, 160 aa, Haemophilus influenzae
MKKIILTLSLGLLTACSAQTQKVEQNDVKLTAPTDVRSGYVRLVKNANYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGKAFSVDKK

SEQ ID NO: 24 - protein, 160 aa, Haemophilus influenzae
MKKIILTLSLGLLTACSAQIQKAEQNDVKLAPPTDVRSGYIRLVKNVNYYIDSESIWVDNQEPQIVHFDAVVNLDKGLYVYPEPKRYARSVRQYKILNCANYHLTQVRIDFYDEFWGQGLRAAPKKQKKHTLSLTPDTTLYNAAQIICANYGKAFSVDKK

SEQ ID NO: 25 - protein, 160 aa, Artificial Sequence; identical to SEQ ID NO: 5
  306543X4

SEQ ID NO: 26 - protein, 160 aa, Haemophilus influenzae; identical to SEQ ID NO: 10

SEQ ID NO: 27 - protein, 160 aa, Haemophilus influenzae; identical to SEQ ID NO: 5

SEQ ID NO: 28 - protein, 160 aa, Haemophilus influenzae; identical to SEQ ID NO: 2

SEQ ID NO: 29 - protein, 160 aa, Haemophilus influenzae; identical to SEQ ID NO: 5

SEQ ID NO: 30 - protein, 160 aa, Artificial Sequence; identical to SEQ ID NO: 21
  27W116791N

SEQ ID NO: 31 - protein, 160 aa, Haemophilus influenzae; identical to SEQ ID NO: 5

SEQ ID NO: 32 - protein, 160 aa, Haemophilus influenzae; identical to SEQ ID NO: 2

SEQ ID NO: 33 - protein, 160 aa, Artificial Sequence; identical to SEQ ID NO: 2
  N162

SEQ ID NO: 34 - protein, 160 aa, Haemophilus influenzae
MKKIILTLSLGLLTACSAQTQKAEQNDVKLAPPTDVRSGYIRLVKNVNYYIDSESIWVDNQEPQIVHFDAVVNLDKGLYVYPEPKRYARSVRQYKILNCANYHLTQIRTDFYDEFWGQGLRAAPKKQKKHTLSLTPDTTLYNAAQIICANYGKAFSVDKK

SEQ ID NO: 35 - protein, 160 aa, Haemophilus influenzae
MKKIILTLSLGLLTACSAQTQKVEQNDVKLTAPADVRSGYVRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGKAFSVDKK

SEQ ID NO: 36 - protein, 159 aa, Haemophilus influenzae
MKKIILTLSLGLLTACSAQIQKAKQNDVKLAPPTDVRSGYIRLVKNVNYYIDSESIWVDNQEPQIVHFDAVVNLDKGLYVYPEPKRYARSVRYKILNCANYHLTQVRTDFYDEFWGQGLRAAPKKQKKHTLSLTPDTTLYNAAQIICANYGEAFSVDKK

SEQ ID NO: 37 - protein, 159 aa, Haemophilus influenzae; identical to SEQ ID NO: 36

SEQ ID NO: 38 - protein, 160 aa, Haemophilus influenzae; identical to SEQ ID NO: 5

SEQ ID NO: 39 - protein, 160 aa, Haemophilus influenzae; identical to SEQ ID NO: 5

SEQ ID NO: 40 - protein, 160 aa, Haemophilus influenzae; identical to SEQ ID NO: 5

SEQ ID NO: 41 - protein, 160 aa, Haemophilus influenzae; identical to SEQ ID NO: 5

SEQ ID NO: 42 - protein, 160 aa, Haemophilus influenzae
MKKIILTLSLGLLTACSAQIQKAEQNDVKLAPPTDVRSGYIRLVKNVNYYIDSESIWVDNQEPQIVHFDTVVNLDKGLYVYPEPKRYARSVRQYKILNCANYHLTQVRTDFYDEFWGQSLRAAPKKQKKHTLSLTPDTTLYNAAQIICANYGKAFSVDKK

SEQ ID NO: 43 - protein, 160 aa, Haemophilus influenzae
MKKIILTLSLGLLTACSAQTQKVEQNDVKLTPPTDVRSGYVRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTVYNAAQIICANYGKAFSVDKK

SEQ ID NO: 44 - protein, 160 aa, Haemophilus influenzae; identical to SEQ ID NO: 43

SEQ ID NO: 45 - protein, 160 aa, Haemophilus influenzae; identical to SEQ ID NO: 2

SEQ ID NO: 46 - protein, 160 aa, Haemophilus influenzae; identical to SEQ ID NO: 10

SEQ ID NO: 47 - protein, 160 aa, Haemophilus influenzae; identical to SEQ ID NO: 10

SEQ ID NO: 48 - protein, 160 aa, Haemophilus influenzae
MKKIILTLSLGLLTACSAQTQKAEQNDVKLTPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGKAFSVDKK

SEQ ID NO: 49 - protein, 160 aa, Haemophilus influenzae
MKKIILTLSLGLLTACSAQIQKAKQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGKAFSVDKK

SEQ ID NO: 50 - protein, 160 aa, Haemophilus influenzae; identical to SEQ ID NO: 10

SEQ ID NO: 51 - protein, 160 aa, Haemophilus influenzae; identical to SEQ ID NO: 34

SEQ ID NO: 52 - protein, 160 aa, Haemophilus influenzae; identical to SEQ ID NO: 2

SEQ ID NO: 53 - protein, 160 aa, Haemophilus influenzae; identical to SEQ ID NO: 5

SEQ ID NO: 54 - protein, 159 aa, Haemophilus influenzae; identical to SEQ ID NO: 9

SEQ ID NO: 55 - protein, 141 aa, Haemophilus influenzae
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 56 - protein, 149 aa, Haemophilus influenzae
MKLTTQQTLKKGFTLIELMIVIAIIAILATIAIPSYQNYTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 57 - protein, 149 aa, Haemophilus influenzae
MKLTTQQTLKKGFTLIELMIVIAIIAILATIAIPSYQNYTKKAAVSELLQASAPYKADVELCVYSTNETTKCTGGKNGIAADITTAKGYVKSVTTSNGAITVAGNGTLDGMSYTLTAEGDSAKGVTWKTTCKGTDASLFPANFCGSVTQ

SEQ ID NO: 58 - protein, 149 aa, Haemophilus influenzae; identical to SEQ ID NO: 56

SEQ ID NO: 59 - protein, 149 aa, Haemophilus influenzae
MKLTTQQTLKKGFTLIELMIVIAIIAILATIAIPSYKNYTKKAAVSELLQASAPYKADVELCVYSTGKPSSCSGGSNGIAADITTAKGYVASVITQSGGITVKGDGTLANMEYILQAAGNAAAGVTWTTTCKGTDASLFPANFCGSVTQ

SEQ ID NO: 60 - protein, 149 aa, Haemophilus influenzae; identical to SEQ ID NO: 56

SEQ ID NO: 61 - protein, 149 aa, Haemophilus influenzae
MKLTTQQTLKKGFTLIELMIVIAIIAILATIAIPSYQNYTKKAAVSELLQASAPYKSDVELCVYSTGKPSTCSGGSNGIAADITTVKGYVKSVTTSNGAITVAGNGTLDGMSYTLTAEGDSAKGVTWTTTCKGTDASLFPANFCGSVTQ

SEQ ID NO: 62 - protein, 149 aa, Haemophilus influenzae; identical to SEQ ID NO: 56

SEQ ID NO: 63 - protein, 149 aa, Haemophilus influenzae; identical to SEQ ID NO: 56

SEQ ID NO: 64 - protein, 149 aa, Haemophilus influenzae; identical to SEQ ID NO: 56

SEQ ID NO: 65 - protein, 149 aa, Haemophilus influenzae
MKLTTQQTLKKGFTLIELMIVIAIIAILATIAIPSYKNYTKKAAVSELLQASAPYKADVELCVYSTNEITNCMGGKNGIAADITTAKGYVASVKTQSGGITVKGDGTLANMEYILQATGNAAAGVTWTTTCKGTDASLFPANFCGSITQ

SEQ ID NO: 66 - protein, 149 aa, Haemophilus influenzae
MKLTTQQTLKKGFTLIELMIVIAIIAILATIAIPSYQNYTKKASVSELLQASAPYKADVELCVYSTNETTNCTGGKNGIAADITTAKGYVASVKTQSGGITVKGNGTLANMEYILQAKGNATAGVTWTTTCKGTDASLFPANFCRSVTK

SEQ ID NO: 67 - protein, 149 aa, Haemophilus influenzae
MKLTTLQTLKKGFTLIELMIVIAIIAILATIAIPSYQNYTKKAAVSELLQASAPYKADVELCVYSTNETTSCTGGKNGIAADIKTAKGYVASVITQSGGITVKGNGTLANMEYILQAKGNAAAGVTWTTTCKGTDASLFPANFCGSVTK

SEQ ID NO: 68 - protein, 149 aa, Haemophilus influenzae
MKLTTQQTLKKGFTLIELMIVIAIIAILATIAIPSYQNYTKKAAVSELLQASAPYKADVELCVYSTGKPSSCSGGSNGIAADITTAKGYVKSVTTSNGAITVKGDGTLANMEYILQASGNAATGVTWTTTCKGTDASLFPANFCGSVTQ

SEQ ID NO: 69 - protein, 149 aa, Haemophilus influenzae; identical to SEQ ID NO: 67

SEQ ID NO: 70 - protein, 149 aa, Haemophilus influenzae
MKLTTLQTLKKGFTLIELMIVIAIIAILATIAIPSYQNYTKKAAVSELLQASAPYKADVELCVYSTGKPSTCSGGSNGIAADITTAKGYVASVKTQSGGITVKGNGTLANMEYILQATGNAATGVTWTTTCKGTDASLFPANFCGSVTQ

SEQ ID NO: 71 - protein, 149 aa, Haemophilus influenzae
MKLTTQQTLKKGFTLIELMIVIAIIAILATIAIPSYQNYTKKASVSELLQASAPYKSDVELCVYSTGKPSTCSGGSNGIAADITTAKGYVASVKTQSGGITVKGNGTLANMEYILQAKGNATAGVTWTTTCKGTDASLFPANFCRSVTK

SEQ ID NO: 72 - protein, 149 aa, Haemophilus influenzae; identical to SEQ ID NO: 56

SEQ ID NO: 73 - protein, 149 aa, Haemophilus influenzae
MKLTTQQTLKKGFTLIELMIVIAIIAILATIAIPSYQNYTKKAAVSELLQASAPYKADVELCVYSTNETTNCTGGKNGIAADITTAKGYVASVKTQSGGITVKGDGTLANMEYILQATGNAATGVTWTTTCKGTEASLFPANFCGSVTQ

SEQ ID NO: 74 - protein, 149 aa, Haemophilus influenzae; identical to SEQ ID NO: 66

SEQ ID NO: 75 - protein, 149 aa, Haemophilus influenzae; identical to SEQ ID NO: 70

SEQ ID NO: 76 - protein, 149 aa, Haemophilus influenzae
MKLTTQQTLKKGFTLIELMIVIAIIAILATIAIPSYQNYTKKAAVSELLQASAPYKADVELCVYSTGKPSTCSGGSNGIAADITTAKGYVASVKTQSGGITVKGNGTLANMEYILQAKGNATAGVTWTTTCKGTDASLFPANFCRSVTK

SEQ ID NO: 77 - protein, 149 aa, Haemophilus influenzae; identical to SEQ ID NO: 73

SEQ ID NO: 78 - protein, 149 aa, Haemophilus influenzae; identical to SEQ ID NO: 73

SEQ ID NO: 79 - protein, 149 aa, Haemophilus influenzae
MKLTTQQTLKKGFTLIELMIVIAIIAILATIAIPSYQNYTKKAAVSELLQASAPYKSDVELCVYSTGKPSTCSGGSNGIAADITTAKGYVKSVTTSNGAITVAGNGTLDGMSYTLTAEGDSAKGVTWKTTCKGTDASLFPANFCGSVTK

SEQ ID NO: 80 - protein, 149 aa, Haemophilus influenzae
MKLTTQQTLKKGFTLIELMIVIAIIAILATIAIPSYQNYTKKAAVSELLQASAPYKADVELCVYSTNEATKCTGGKNGIAADITTAKGYVKSVTTSNGAITVKGDGTLANMEYILQASGNAATGVTWTTTCKGTDASLFPANFCGSVTQ

SEQ ID NO: 81 - protein, 149 aa, Haemophilus influenzae; identical to SEQ ID NO: 80

SEQ ID NO: 82 - protein, 149 aa, Haemophilus influenzae; identical to SEQ ID NO: 56

SEQ ID NO: 83 - protein, 149 aa, Haemophilus influenzae
MKLTTQQTLKKGFTLIELMIVIAIIAILATIAIPSYQNYTKKAAISELLQASAPYKSDVELCVYSTGKPSTCSGGSNGIAADITTAKGYVASVKTQSGGITVKGNGTLANMEYILQAKGNATAGVTWTTTCKGTDASLFPANFCRSVTK

SEQ ID NO: 84 - protein, 149 aa, Haemophilus influenzae; identical to SEQ ID NO: 73

SEQ ID NO: 85 - protein, 149 aa, Haemophilus influenzae; identical to SEQ ID NO: 56

SEQ ID NO: 86 - protein, 149 aa, Haemophilus influenzae; identical to SEQ ID NO: 61

SEQ ID NO: 87 - protein, 149 aa, Haemophilus influenzae; identical to SEQ ID NO: 56

SEQ ID NO: 88 - protein, 149 aa, Haemophilus influenzae
MKLTTQQTLKKGFTLIELMIVIAIIAILATIAIPSYQNYTKKAAVSELLQASAPYKADVELCVYSTNETTNCTGGKNGIAADITTAKGYVASVKTQSGGITVKGNGTLANMEYILQATGNAATGVTWTTTCKGTDASLFPANFCGSVTQ

SEQ ID NO: 89 - protein, 149 aa, Haemophilus influenzae
MKLTTQQTLKKGFTLIELMIVIAIIAILATIAIPSYQNYTKKAAVSELLQASAPYKADVELCVYSTGKPSTCSGGSNGIAADITTAKGYVKSVTTSNGAITVAGNGTLDRMSYTLTAEGDSAKGVTWTTTCKGTDASLFPANFCGSVTQ

SEQ ID NO: 90 - protein, 149 aa, Haemophilus influenzae; identical to SEQ ID NO: 68

SEQ ID NO: 91 - protein, 149 aa, Haemophilus influenzae
MKLTTQTTLKKGFTLIELMIVIAIIAILATIAIPSYQNYTKKAAVSELLQASAPYKSDVELCVYSTNETTNCTGGKNGIAADITTAKGYVASVITQSGGITVKGNGTLTNMEYILQATGNAATGVTWTTTCKGTDASLFPANFCGSITQ

SEQ ID NO: 92 - protein, 149 aa, Haemophilus influenzae
MKLTTLQTLKKGFTLIELMIVIAIIAILATIAIPSYQNYTKKAAVSELLQASAPYKADVELCVYSTGKPSTCSGGNNGIAADIKTAKGYVASVKTQSGGITVKGDGTLANMEYILQATGNAATGVTWTTTCKGTDASLFPANFCGSVTQ

SEQ ID NO: 93 - protein, 149 aa, Haemophilus influenzae; identical to SEQ ID NO: 68

SEQ ID NO: 94 - protein, 149 aa, Haemophilus influenzae
MKLTTQQTLKKGFTLIELMIVIAIIAILATIAIPSYQNYTKKAAVSELLQASAPYKADVELCVYSTNEATKCTGGKNGIAADITTAKGYVKSVTTSNGAITVKGDGTLANMEYILQASGNAATGVTWTTTCKGTDTSLFPANFCGSVTQ

SEQ ID NO: 95 - protein, 149 aa, Haemophilus influenzae; identical to SEQ ID NO: 56

SEQ ID NO: 96 - protein, 149 aa, Haemophilus influenzae; identical to SEQ ID NO: 88

SEQ ID NO: 97 - protein, 149 aa, Haemophilus influenzae; identical to SEQ ID NO: 76

SEQ ID NO: 98 - protein, 149 aa, Haemophilus influenzae; identical to SEQ ID NO: 76

SEQ ID NO: 99 - protein, 149 aa, Haemophilus influenzae; identical to SEQ ID NO: 56

SEQ ID NO: 100 - protein, 149 aa, Haemophilus influenzae; identical to SEQ ID NO: 76

SEQ ID NO: 101 - protein, 149 aa, Haemophilus influenzae; identical to SEQ ID NO: 80

SEQ ID NO: 102 - protein, 149 aa, Haemophilus influenzae; identical to SEQ ID NO: 56

SEQ ID NO: 103 - protein, 149 aa, Haemophilus influenzae; identical to SEQ ID NO: 56

SEQ ID NO: 104 - protein, 149 aa, Haemophilus influenzae
  misc_feature (116)..(116): Xaa can be any naturally occurring amino acid
MKLTTQQTLKKGFTLIELMIVIAIIAILATIAIPSYQNYTKKAAVSELLQASAPYKADVELCVYSTNETTNCTGGKNGIAADITTAKGYVKSVTTSNGAITVKGDGTLANMEYILXATGNAATGVTWTTTCKGTDASLFPANFCGSVTQ

SEQ ID NO: 105 - protein, 149 aa, Haemophilus influenzae; identical to SEQ ID NO: 56

SEQ ID NO: 106 - protein, 149 aa, Haemophilus influenzae; identical to SEQ ID NO: 56

SEQ ID NO: 107 - protein, 149 aa, Haemophilus influenzae; identical to SEQ ID NO: 92

SEQ ID NO: 108 - protein, 149 aa, Haemophilus influenzae; identical to SEQ ID NO: 92

SEQ ID NO: 109 - protein, 149 aa, Haemophilus influenzae
MKLTTQQTLKKGFTLIELMIVIAIIAILATIAIPSYQNYTKKAAVSELLQASAPYKADVELCVYSTNETTSCTGGKNGIAADITTAKGYVKSVTTSNGAITVAGNGTLDGMSYTLTAEGDSAKGVTWKTTCKGTDASLFPANFCGSVTQ

SEQ ID NO: 110 - protein, 149 aa, Haemophilus influenzae; identical to SEQ ID NO: 109

SEQ ID NO: 111 - protein, 149 aa, Haemophilus influenzae; identical to SEQ ID NO: 92

SEQ ID NO: 112 - protein, 149 aa, Haemophilus influenzae; identical to SEQ ID NO: 92

SEQ ID NO: 113 - protein, 149 aa, Haemophilus influenzae; identical to SEQ ID NO: 92

SEQ ID NO: 114 - protein, 149 aa, Haemophilus influenzae; identical to SEQ ID NO: 73

SEQ ID NO: 115 - protein, 149 aa, Haemophilus influenzae; identical to SEQ ID NO: 92

SEQ ID NO: 116 - protein, 149 aa, Haemophilus influenzae; identical to SEQ ID NO: 76

SEQ ID NO: 117 - protein, 149 aa, Haemophilus influenzae; identical to SEQ ID NO: 80

SEQ ID NO: 118 - protein, 149 aa, Haemophilus influenzae; identical to SEQ ID NO: 70

SEQ ID NO: 119 - protein, 149 aa, Haemophilus influenzae; identical to SEQ ID NO: 71

SEQ ID NO: 120 - protein, 110 aa, Haemophilus influenzae
TKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 121 - protein, 275 aa, Haemophilus influenzae
MKYLLPTAAAGLLLLAAQPAMAIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 122 - protein, 253 aa, Haemophilus influenzae
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 123 - DNA, 361 bp, Artificial Sequence
  Antibody VH Chain
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttctctttggaaacctctgccagcactgcctatttgcagatcaacaacctcaaaaatgaggacacggctacatatttctgtgcaaggggagggtacccctcctcgcgggcgcccccttactggggccaagggactctggtcactgtctctgcag

SEQ ID NO: 124 - protein, 120 aa, Artificial Sequence
  Antybody VH Chain
QIQLVQSGPELKKPGETVKISCKASGYTFTNYGMNWVKQAPGKGLKWMGWINTYTGEPTYADDFKGRFAFSLETSASTAYLQINNLKNEDTATYFCARGGYPSSRAPPYWGQGTLVTVSA

SEQ ID NO: 125 - protein, 5 aa, Artificial Sequence
  Antibody VH Chain CDRs
NYGMN

SEQ ID NO: 126 - protein, 17 aa, Artificial Sequence
  Antibody VH Chain CDRs
WINTYTGEPTYADDFKG

SEQ ID NO: 127 - protein, 11 aa, Artificial Sequence
  Antibody VH Chain CDRs
GGYPSSRAPPY

SEQ ID NO: 128 - DNA, 321 bp, Artificial Sequence
  Antibody VL Chain
gacattgtgatgacccagtctcacaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatccattcacgttcggctcggggacaaagttggaaataaaa

SEQ ID NO: 129 - protein, 107 aa, Artificial Sequence
  Antibody VL Chain
DIVMTQSHKFMSTSVGDRVSITCKASQDVGTAVAWYQQKPGQSPKLLIYWASTRHTGVPDRFTGSGSGTDFTLTISNVQSEDLADYFCQQYSSYPFTFGSGTKLEIK

SEQ ID NO: 130 - protein, 11 aa, Artificial Sequence
  Antibody VL Chain CDRs
KASQDVGTAVA

SEQ ID NO: 131 - protein, 7 aa, Artificial Sequence
  Antibody VL Chain CDRs
WASTRHT

SEQ ID NO: 132 - protein, 9 aa, Artificial Sequence
  Antibody VL Chain CDRs
QQYSSYPFT

SEQ ID NO: 133 - protein, 9 aa, Haemophilus influenzae
IRLVKNVNY

SEQ ID NO: 134 - protein, 14 aa, Haemophilus influenzae
YNAAQIICANYGEA

SEQ ID NO: 135 - protein, 20 aa, Haemophilus influenzae
CVYSTNETTNCTGGKNGIAA

SEQ ID NO: 136 - protein, 25 aa, Haemophilus influenzae
PKRYARSVRQYKILNCANYHLTQVR

SEQ ID NO: 137 - protein, 28 aa, Haemophilus influenzae
IRLVKNVNYYIDSESIWVDNQEPQIVHF

SEQ ID NO: 138 - protein, 360 aa, Haemophilus influenzae
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFL

SEQ ID NO: 139 - protein, 348 aa, Haemophilus influenzae
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 140 - DNA, 989 bp, Haemophilus influenzae
gcgaatacccaaatgaaatcagacaaaatcattattgctcaccgtggtgctagcggttatttaccagagcatacgttagaatctaaagcacttgcgtttgcacaacacgcagattatttagagcaagatttagcaatgactaaggatggtcgtttagtggttattcacgatcactttttagatggcttgactgatgttgcgaaaaaattcccacatcgtcaccgtaaagatggtcgttactatgtcatcgactttaccttaaaagaaattcaaagtttagaaatgactgaaaactttgaaaccaaagacggcaaacaagcgcaagtttatcctaatcgtttcccactttggaaatcacattttagaattcacacctttgaagatgaaattgagtttatccaaggcttagaaaaatcgactggcagaaaagtagggatttatccagaaatcaaagcaccttggttccaccatcaaaatggcaaagatattgcagctgaaacgctcaaagtgttaaaaaaatatggctatgataagaaaaccgatatggtttacttacaaactttcgattttaatgaattaaaacgtatcaaaacggaattacttccacaaatgggaatggatttaaaattagttcaattaattgcttatacagattggaaagaaacacaagaaaaagacccaaagggttattgggtaaactataattacgattggatgtttaaacctggtgcaatggcagaagtggttaaatatgccgatggtgttggcccaggttggtatatgttagttaataaagaagaatccaaacctgataatattgtgtacactccgttggtaaaagaacttgcacaatataatgtggaagtgcatccttacaccgtgcgtaaagatgcactgcccgagtttttcacagacgtaaatcaaatgtatgatgccttattgaataaatcaggggcaacaggtgtatttactgatttcccagatactgg

SEQ ID NO: 141 - DNA, 989 bp, Haemophilus influenzae; identical to SEQ ID NO: 140

SEQ ID NO: 142 - DNA, 989 bp, Haemophilus influenzae
gcaaatacccaaatgaaatctgacaaaatcatcattgctcatcgtggtgctagcggttatttaccagagcatacgttagaatctaaagcacttgcgtttgcacagcacgctgattacttagagcaagatttagcaatgactaaggatggtcgtttagtggttattcacgatcactttttagatggcttgactgatgttgcgaaaaaattcccacatcgtcaccgtaaagatggtcgttactatgtcatcgactttaccttaaaagaaattcaaagtttagaaatgacagaaaactttgaaaccaaagatggcaaacagacacaagtttatcctaatcgtttccccctttggcaatcccatttccgtattcacacctttgaagatgaaattgaatttattcaaggtttagaaaaatcgacgggcaaaaaagtagggatttatccagaaatcaaagcaccttggttccaccatcaaaatggcaaagatattgctgctgaaacgctcaaagtgttaaaaaaatatggctatgataagaaaaccgatatggtttacttacaaactttcgattttaatgaattaaaacgtatcaaaacggaattacttccacaaatgggtatggatttgaaattagttcaattaattgcttatacagattggaaagaaacacaagaaaaagattcaaagggttattgggtaaactataattacgattggatgtttaaacctggtgcaatggcagaagtggttaaatatgccgatggtgttggcccaggttggtatatgttagttaataaagaagaatccaaacctgataatattgtgtacactccgttggtaaaagaacttgcacaatataatgtggaagtgcatccttacaccgtgcgtaaagatgcactacctgcgtttttcacagacgtaaatcaaatgtatgatgccttattgaataaatcaggggcaacaggtgtatttactgatttcccagatactgg

SEQ ID NO: 143 - DNA, 1259 bp, Haemophilus influenzae
acctacggtactaaataattagcttaaaaaaggcggcgggcaaattgcttagtcgccttttttgtaactaaaatctaaaaaaaaccataaaaatttaccgcactcttaaggagaaaatacttatgaaacttaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcaccgtaaagatggccgttactatgtcatcgactttaccttaaaagaaattcaaagtttagaaatgacagaaaactttgaaaccaaagatggcaaacaagcgcaagtttatcctaatcgtttcccactttggaaatcacattttagaattcacacctttgaagatgaaattgaatttatccaaggcttagaaaaatccactggcaaaaaagtagggatttatccagaaatcaaagcaccttggttccaccatcaaaatggtaaagatattgctgctgaaacgctcaaagtgttaaaaaaatatggctatgataagaaaaccgatatggtttacttacaaactttcgattttaatgaattaaaacgtatcaaaacggaattacttccacaaatgggtatggatttgaaattagttcaattaattgcttatacagattggaaagaaacacaagaaaaagatccaaagggttattgggtaaactataattacgattggatgtttaaacctggagcaatggcagaagtggttaaatatgccgatggtgttggtccaggttggtatatgttagttaataaagaagaatccaaacctgataatattgtgtacactccgttggtaaaagaacttgcacaatataatgtggaagtgcatccttacaccgtgcgtaaagatgcactacccgcgtttttcacagatgtaaatcaaatgtatgatgccttattgaataaatcaggggcaacaggtgtatttactgatttcccagatactggcgtggaattcttaaaaggaataaaataatatccctcacaaccgtgggtaaacatacccacgttaactagg

SEQ ID NO: 144 - DNA, 1258 bp, Haemophilus influenzae
acttacggtactaaataattagcttaaaaaaggcggtgggtaaattgcttagtcgccttttttgtaactaaaatctaaaaaaaccataaaaatttaccgcactcttaaggagaaaatacttatgaaacttaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcaccgtaaagatggtcgttactatgtcatcgactttaccttaaaagaaattcaaagtttagaaatgacagaaaactttgaaaccaaagacggcaaacaagcgcaagtttatcctaatcgtttcccactttggaaatcacattttagaattcatacctttgaagatgaaattgaatttatccaaggcttagaaaaatccactggcaaaaaagtagggatttatccagaaatcaaagcaccttggttccaccatcaaaatggtaaagatattgctgctgaaacgctcaaagtgttaaaaaaatatggctatgataagaaaaccgatatggtttacttacaaactttcgattttaatgaattaaaacgtatcaaaacggaattacttccacaaatggggatggatttgaaattagttcaattaattgcttatacagattggaaagaaacacaagaaaaagacccaaagggttattgggtaaactataattacgattggatgtttaaacctggagcaatggcagaagtggttaaatatgccgatggtgttggtccaggttggtatatgttagttaataaagaagaatccaaacctgataatattgtgtacactccgttggtaaaagaacttgcacaatataatgtggaagtgcatccttacaccgtgcgtaaagatgcactgcccgagtttttcacagacgtaaatcaaatgtatgatgtcttattgaataaatcaggggcaacaggtgtatttactgatttcccagatactggcgtggaattcttaaaaggaataaaataatatccctcacaaccgtgggtaaacatacccacgttaactagg

SEQ ID NO: 145 - DNA, 1252 bp, Haemophilus influenzae
acttacggtactaaataattagcttaaaaaaggcggtgggcaaattgcttagtcgccttttttgtaactaaaatctaaaaaaaccataaaaatttaccgcactttcaaggagaaaatacttatgaaacttaaaactttagccctttctttattagcagctggcgtactagcaggttgtagcagccattcatcaaatatggcgaaaacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcaccgtaaagatggtcgttactatgtcatcgactttaccttaaaagaaattcaaagtttagaaatgacagaaaactttgaaaccaaagacggcaaacaagcgcaagtttatcctaatcgtttccccctttggcaatcccatttccgtattcacacctttgaagatgaaattgaatttatccaaggcttagaaaaatcgactggcagaaaagtagggatttatccagaaatcaaagcaccttggttccaccatcaaaatggtaaagatattgctgctgaaacgctcaaagtgttgaaaaaatatggctatgataagaaaaccgatatggtttacttacaaactttcgactttaatgaattaaaacgtatcaaaacggaattacttccacaaatgggtatggatttgaaattagttcaattaattgcttatacagattggaaagaaacacaagaaaaagattcaaagggttattgggtaaactataattacgattggatgtttaaacctggtgcaatggcagaagtggttaaatatgccgatggtgttggcccaggttggtatatgttagttaataaagaagaatccaaacctgataatattgtgtacactccgttggtaaaagaacttgcaaaatataatgtggaagtgcatccttacaccgtgcgtaaagatgcactgcctgcgtttttcacagacgtaaatcaaatgtatgatgctttattgaataaatcaggggcaacaggtgtatttactgatttcccagatactggcgtggaattcttaaaaggaatagaataatatccctcacaaccgtgggtaaacatacccacggtt

SEQ ID NO: 146 - DNA, 1864 bp, Haemophilus influenzae
gatcggcggtggcgtattagcggtgttattactcttaatcgtaatggttgaagaaggaaaacacaaagcgaaattaggcgatacttacggtactaaataattagcttaaaaaaggcggtgggcaaattgcttagtcgccttttttgtaactaaaatctaaaaactctataaaaatttaccgcactcttaaggagaaaatacttatgaaacttaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccaaagatggcaaacaagcgcaagtttatcctaatcgtttccctctttggaaatcacattttagaattcatacctttgaagatgaaattgaatttatccaaggcttagaaaaatccactggcaaaaaagtagggatttatccagaaatcaaagcaccttggttccaccatcaaaatggtaaagatattgctgctgaaacgctcaaagtgttaaaaaaatatggctatgataagaaaaccgatatggtttacttacaaactttcgattttaatgaattaaaacgtatcaaaacggaattacttccacaaatgggaatggatttgaaattagttcaattaattgcttatacagattggaaagaaacacaagaaaaagacccaaagggttattgggtaaactataattacgattggatgtttaaacctggtgcaatggcagaagtggttaaatatgccgatggtgttggcccaggttggtatatgttagttaataaagaagaatccaaacctgataatattgtgtacactccgttggtaaaagaacttgcacaatataatgtggaagtgcatccttacaccgtgcgtaaagatgcactgcccgagtttttcacagacgtaaatcaaatgtatgatgccttattgaataaatcaggggcaacaggtgtatttactgatttcccagatactggcgtggaattcttaaaaggaataaaataatatccctcacaaccgtgggtaaacatacccacggttaactaggtttctatatcgtagaaactaaaaatctactctaacagagtaacatcataatcaatctaggtgttctaacctagaattcaaataaggaggctatttcaaaacactccgtattcttttttaataaattctcttccctttacttagggaaaacactcttcatttcaaccgcacttctaaggagtgctctatggataaatcattaaaagcgaactgtattggcgagtttttaggtacagccttattgattttctttggtgtgggctgcgttgcagcactaaaagtagcaggcgctagttttggcttgtgggaaatcagcattatgtgggggatgggcgttgcacttgcagtatatgcaacagcgggtttatctggcgcacatttaaaccctgcagtaaccattgccctttggaaatttgcttgctttgatggcaaaaaagtaattccttacatcatttcacaaatgctcggcgcattctttgctgccgcattagtttatgccttataccgcaatgtttttatcgatc

SEQ ID NO: 147 - protein, 10 aa, Haemophilus influenzae
SSHSSNMANT

SEQ ID NO: 148 - protein, 630 aa, Moraxella catarrhalis
MKTMKLLPLKIAVTSAMIIGLGAASTANAQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 149 - protein, 520 aa, Moraxella catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKASHHHHHH

SEQ ID NO: 150 - protein, 512 aa, Moraxella catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTK

SEQ ID NO: 151 - protein, 513 aa, Moraxella catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKH

SEQ ID NO: 152 - protein, 514 aa, Moraxella catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKHH

SEQ ID NO: 153 - protein, 499 aa, Moraxella catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSASHHHHHH

SEQ ID NO: 154 - protein, 491 aa, Moraxella catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKS

SEQ ID NO: 155 - protein, 544 aa, Moraxella catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAASHHHHHH

SEQ ID NO: 156 - protein, 538 aa, Moraxella catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 157 - protein, 537 aa, Moraxella catarrhalis
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 158 - protein, 536 aa, Moraxella catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAA

SEQ ID NO: 159 - protein, 519 aa, Moraxella catarrhalis
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKASHHHHHH

SEQ ID NO: 160 - DNA, 358 bp, Artificial Sequence
  PilA mAb VH Region
cagggtcagctgcagcagtctggacctgagctggtgaagcctggggcttcagtgaagatgtcctgcaaggcttctggatacacattcactgactatgttataagctgggtgaagcagagaattggacagggccttgagtggattggagagattcatcctggaagtggtagtattcactacaatgagaagttcaagggcaaggccacactgactgcagacaaatcctccaacacagcctacatgcagctcagcagcctgacatctgaggactctgcggtctatttctgtgcaagaagggggttacgacgtccctggtttgcttactggggccaagggactctggtcactgtctctgcag

SEQ ID NO: 161 - protein, 119 aa, Artificial Sequence
  PilA mAb VH Region
QGQLQQSGPELVKPGASVKMSCKASGYTFTDYVISWVKQRIGQGLEWIGEIHPGSGSIHYNEKFKGKATLTADKSSNTAYMQLSSLTSEDSAVYFCARRGLRRPWFAYWGQGTLVTVSA

SEQ ID NO: 162 - DNA, 333 bp, Artificial Sequence
  PilA mAb VL Region
gacattgtgctgacacagtctcctgcttccttagctgtatctctggggcagagggccaccatctcatgcagggccagcaaaagtgtcagtacatctggctatagttatatgcactggttccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgctcacgttcggtgctgggaccaagctggagctgaaa

SEQ ID NO: 163 - protein, 111 aa, Artificial Sequence
  PilA mAb VL Region
DIVLTQSPASLAVSLGQRATISCRASKSVSTSGYSYMHWFQQKPGQPPKLLIYLASNLESGVPARFSGSGSGTDFTLNIHPVEEEDAATYYCQHSRELPLTFGAGTKLELK

SEQ ID NO: 164 - protein, 5 aa, Artificial Sequence
  CDR-H1
DYVIS

SEQ ID NO: 165 - protein, 17 aa, Artificial Sequence
  CDR-H2
EIHPGSGSIHYNEKFKG

SEQ ID NO: 166 - protein, 10 aa, Artificial Sequence
  CDR-H3
RGLRRPWFAY

SEQ ID NO: 167 - protein, 15 aa, Artificial Sequence
  CDR-L1
RASKSVSTSGYSYMH

SEQ ID NO: 168 - protein, 7 aa, Artificial Sequence
  CDR-L2
LASNLES

SEQ ID NO: 169 - protein, 9 aa, Artificial Sequence
  CDR-L3
QHSRELPLT

(19) United States
(12) Patent Application Publication
(10) Pub. No.: US 2022/0221455 A1
(43) Pub. Date: Jul. 14, 2022

(54) ANTIGEN BINDING PROTEINS AND ASSAYS

(71) Applicant: GLAXOSMITHKLINE BIOLOGICALS SA, Rixensart (DE)

(72) Inventors: Nathalie NORAIS, Siena (IT); Silvia ROSSI PACCANI, Siena (IT); Simona RONDINI, Siena (IT)

(73) Assignee: GLAXOSMITHKLINE BIOLOGICALS SA, Rixensart (BE)

(21) Appl. No.: 17/604,008

(22) PCT Filed: Apr. 16, 2020
(86) PCT No.: PCT/EP2020/060647

§ 371 Date: Oct. 15, 2021

(30) Foreign Application Priority Data
Apr. 18, 2019 (EP) 19170340.4
Nov. 22, 2019 (EP) 19210869.4

(51) Int. Cl. G01N 33/569

(52) U.S. Cl. CPC G01N 33/56911; G01N 2800/14; G01N 2800/12; G01N 2333/285

(57) ABSTRACT

The present invention relates to the field of antigen binding proteins and the use of such antigen binding proteins in an assay. More particularly, it relates to antigen binding proteins which bind to an epitope of Protein E and antigen binding proteins which bind to an epitope of PilA. The present invention also relates to assays (particularly in vitro assays) for assessing binding to Protein E and/or PilA and the potency of vaccines containing Protein E and/or PilA. In particular the invention relates to in vitro relative potency assays used in the release of a vaccine to the public.

### TECHNICAL FIELD

The present invention relates to the field of antigen binding proteins and the use of such antigen binding proteins in an assay. More particularly, it relates to antigen binding proteins which bind to an epitope of Protein E and antigen binding proteins which bind to an epitope of PilA. The present invention also relates to assays (particularly in vitro assays) for assessing binding to Protein E and/or PilA and the potency of vaccines containing Protein E and/or PilA. In particular the invention relates to in vitro relative potency assays used in the release of a vaccine to the public.

### BACKGROUND

Protein E (PE) is an outer membrane lipoprotein with adhesive properties. It plays a role in the adhesion/invasion of non-typeable Haemophilus influenzae (NTHi) to epithelial cells [J. Immunology 183: 2593-2601 (2009); The Journal of Infectious Diseases 199:522-531 (2009), Microbes and Infection 10:87-96 (2008)]. It is highly conserved in both encapsulated Haemophilus influenzae and non-typeable H. influenzae and has a conserved epithelial binding domain [The Journal of Infectious Diseases 201:414-419 (2010)]. Thirteen different point mutations have been described in different Haemophilus species when compared with Haemophilus influenzae Rd as a reference strain. Its expression is observed on both logarithmic growing and stationary phase bacteria. [WO2007/084053].

Protein E is also involved in human complement resistance through binding to extracellular matrix proteins vitronectin and laminin [Immunology 183: 2593-2601 (2009)]. Protein E, by the binding domain of SEQ ID NO: 136 (corresponding to amino acids 84-108 of SEQ ID NO. 1), binds vitronectin which is an important inhibitor of the terminal complement pathway [J. Immunology 183:2593-2601 (2009)]. In addition, Protein E is able to directly interact with the extracellular matrix protein laminin which is present on lung epithelial cells. The laminin binding region, of SEQ ID NO: 137 (i.e. residues 41-68 of SEQ ID NO: 1) is substantially exposed whilst the vitronectin binding site is only partially accessible. A Protein E epitope is reported herein which differs from any epitopes previously reported and has been demonstrated to be functional in respect of inhibiting laminin binding.

Pilin A (PilA) is likely the major pilin subunit of H. influenzae Type IV Pilus (Tfp) involved in twitching motility [Infection and Immunity, 73: 1635-1643 (2005)]. NTHi PilA is a conserved adhesin expressed in vivo. It has been shown to be involved in NTHi adherence, colonization and biofilm formation. [Molecular Microbiology 65: 1288-1299 (2007)]. Novotny et al 2009 (Vaccine 28(1): 279-289) mapped immunodominant regions of PilA during the design of novel vaccine candidates, however the PilA epitope described herein differs from any epitopes previously reported and has been demonstrated to be functional in respect of biofilm inhibition.

Non-typeable Haemophilus influenzae is an important and common respiratory pathogen that causes otitis media in infants and children. NTHi is, after Streptococcus pneumoniae, the most common cause of acute otitis media in children [J. Immunology 183: 2593-2601 (2009), Pediatrics 113:1451-1465 (2004)]. It is an important cause of sinusitis in children and adults [Current Infectious Disease Reports 11:177-182 (2009)]. It has been associated with increased risk of exacerbations in chronic obstructive pulmonary disease (COPD) in adults [Journal of Chronic Obstructive Pulmonary Disease 3:109-115 (2006)]. In addition, non-typeable H. influenzae causes community-acquired pneumonia in adults and may cause pneumonia in children in developing countries [Current Infectious Disease Reports 11:177-182 (2009)].

Chronic Obstructive Pulmonary Disease (COPD), a common preventable disease, is characterised by persistent airflow limitation that is usually progressive. The airflow limitation is associated with an enhanced chronic inflammatory response in the airways and lungs to noxious particles of gases. It is a multi-component disease that manifests as an accelerated decline in lung function, with symptoms such as breathlessness on physical exertion, deteriorating health status and exacerbations.

Acute exacerbations and comorbidities contribute to the overall disease severity in individual COPD patients. An acute exacerbation of COPD (AECOPD) is an acute event characterised by a worsening of the patient's respiratory symptoms that is beyond normal day-to-day variations and leads to a change in medication [Perez A C, Murphy T F. Potential impact of a Moraxella catarrhalis vaccine in COPD. Vaccine. 2017]. AECOPD increases morbidity and mortality, leading to faster decline in lung function, poorer functional status [Sapey E, Stockley R A. COPD exacerbations. 2: aetiology. Thorax. 2006; 61(3):250-8)]. The lungs are known to be colonised with different species of bacteria [Erb-Downward J R, et al. PLoS One. 2011; 6(2):e16384 and Wilkinson T M A, et al. Thorax. 2017; 72(10):919-27]. In COPD patients, acquisition of new bacterial strains is believed to be an important cause of AECOPD [Sethi S, et al. N Engl J Med. 2002; 347(7):465-71]. Although estimates vary widely, Non-Typeable Haemophilus influenzae (NTHi) appears to be the main bacterial pathogen associated with AECOPD (11-38%), followed by Moraxella catarrhalis (3-25%) and Streptococcus pneumoniae (4-9%) [Alamoudi O S. et al. Respirology. 2007; 12(2):283-7, Bandi V, et al. FEMS Immunol Med MicrobioL 2003; 37(1):69-75, Beasley V, et al. Int J Chron Obstruct Pulmon Dis. 2012; 7:555-69].

Vaccines normally require the manufacturer to test each batch prior to its release for public use. It is desirable to provide an in vitro test since historically in vivo release assays were used which require immunization of many animals. Furthermore, in vitro assays are more sensitive (in terms of detecting marginal effects on vaccine batches) than in vivo studies. Suitable assessments may include potency, structure or immunogenicity. Suitably, such in vitro assay could be used to confirm that a particular vaccine will be expected to have in vivo activity in human recipients. Therefore, there is a need to provide an in vitro assay for assessing the potency of vaccines containing Protein E and/or PilA.

### SUMMARY OF THE INVENTION

The present invention provides antigen binding proteins which bind to an epitope of Protein E and antigen binding proteins which bind to an epitope of PilA. The present invention also relates to assays (particularly in vitro assays) for assessing binding to Protein E and/or PilA and the potency of vaccines containing Protein E and/or PilA. The assays use antigen binding proteins which bind to Protein E or PilA, in particular monoclonal antibodies which functionally inhibit Protein E or PilA function (i.e. inhibit laminin binding or biofilm formation respectively) and/or which recognise epitopes within the Protein E protein (e.g. a conformational epitope within the Protein E protein) or the PilA protein. By comparing the results of a test sample with those obtained using a standard or reference sample of known potency, it is possible to determine the relative potency of the test sample. This can be used for determining whether a manufactured batch of a vaccine is suitable for release to the public, or whether it has experienced a production failure and so should not be used.

Accordingly, in a first aspect of the invention there is provided an antigen binding protein which binds to Protein E at one or more of amino acid residues within I41 to Y49 (e.g. SEQ ID NO: 133) and Y141 to A154 (e.g. SEQ ID NO: 134) of Protein E.

According to a further aspect of the invention, there is provided an antigen binding protein which binds to PilA at one or more of amino acid residues within C62 to A81 (e.g. SEQ ID NO: 135) of PilA

According to a further aspect of the invention, there is provided an immunogenic composition comprising the PE antigen binding protein of the invention and/or the PilA antigen binding protein of the invention.

According to a further aspect of the invention, there is provided a vaccine comprising the PE antigen binding protein of the invention and/or the PilA antigen binding protein of the invention.

According to a further aspect of the invention, there is provided an antigen binding protein, an immunogenic composition or a vaccine of the invention for use in therapy.

According to a further aspect of the invention, there is provided an antigen binding protein, an immunogenic composition or a vaccine of the invention for use in treating or preventing an infection, disease or condition caused by H. influenzae.

According to a further aspect of the invention, there is provided an antigen binding protein, an immunogenic composition or a vaccine of the invention for use in treating or preventing an infection, disease or condition which is otitis media, pneumonia and/or acute exacerbations of chronic obstructive pulmonary disease (AECOPD).

According to a further aspect of the invention, there is provided an antigen binding protein, an immunogenic composition or a vaccine of the invention for use in treating or preventing an infection, disease or condition caused by H. influenzae, in a mammal, particularly a human.

According to a further aspect of the invention, there is provided an antigen binding protein, an immunogenic composition or a vaccine of the invention for use in the treatment or prevention of acute otitis media, pneumonia and/or acute exacerbations of chronic obstructive pulmonary disease (AECOPD).

According to a further aspect of the invention, there is provided an antigen binding protein, an immunogenic composition or a vaccine of the invention for use in the manufacture of a medicament for the treatment or prevention of an infection, disease or condition caused by H. influenzae.

According to a further aspect of the invention, there is provided an antigen binding protein, an immunogenic composition or a vaccine of the invention for use in the manufacture of a medicament for the treatment or prevention of pneumonia, otitis media and/or acute exacerbations of chronic obstructive pulmonary disease AECOPD.

According to a further aspect of the invention, there is provided a method of treatment or prevention of an infection, disease or condition caused by H. influenzae, in a subject in need thereof comprising administering to said subject a therapeutically effective amount of an antigen binding protein, immunogenic composition or vaccine of the invention.

According to a further aspect of the invention, there is provided a method of treatment or prevention of acute exacerbations of chronic obstructive pulmonary disease (AECOPD), pneumonia and/or otitis media in a subject in need thereof comprising administering to said subject a therapeutically effective amount of an antigen binding protein, immunogenic composition or vaccine of the invention.

According to a further aspect of the invention, there is provided the use of the PE antigen binding protein of the invention in the detection of, or measurement of a change in, the conformation of Protein E.

According to a further aspect of the invention, there is provided the use of the PilA antigen binding protein of the invention in the detection of, or measurement of a change in, the conformation of PilA.

According to a further aspect of the invention, there is provided an assay comprising exposing a sample of a test antigen to an antigen binding protein of the invention and measuring the amount of antigen binding protein bound to the test antigen.

According to a further aspect of the invention, there is provided a kit to (i) detect, measure the levels of, and/or measure a change in the conformation of a test antigen or (ii) determine potency of a test antigen, comprising: reagents for preparing an assay mixture, an antigen binding protein of the invention, and optionally instructions for use thereof.

According to a further aspect of the invention, there is provided a method for in vitro analysis of a test antigen, comprising steps of: (i) performing the assay of the invention on a test antigen and a reference sample of known potency; and (ii) comparing the results from step (i) to determine the potency of the test antigen relative to the reference sample.

According to a further aspect of the invention, there is provided a method for analysing a batch of vaccine, comprising steps of: (i) assaying a test antigen taken from a batch of vaccine by the method of the invention; and, if the results of step (i) indicate an acceptable relative potency, (ii) releasing vaccine from the batch for in vivo use.

### DESCRIPTION OF FIGURES

FIG. 1: Biofilm Inhibition. Two anti-PilA monoclonal antibodies (PEPilA/3 and PEPilA/4), the anti-PE ProtE/5 mAb and the positive control (rabbit anti-PilA serum) were shown to prevent biofilm formation. No inhibition of biofilm formation was observed with anti-PE ProtE/3 and the negative control anti-PS19A from Streptococcus pneumoniae mAbs.

FIG. 2: Binding of Protein E, PilA and PE-PilA DSs (Drug Substances) to vitronectin and laminin by SPR (Biacore) assay. Only Protein E binds to vitronectin (A) and laminin (B). No binding was observed with either PilA or the PE-PilA fusion protein LVL-735 (SEQ ID NO: 122).

FIG. 3: Binding of ProtE/5 mAb to Bound Protein E by SPR (Biacore) assay. The ProtE/5 mAb was able to bind to Protein E even once Protein E was already bound to both vitronectin (top) and laminin (bottom).

FIG. 4: ProtE/5 mAb inhibits binding of PE to laminin. ProtE/5 mAb complexes were formed in solution and used as analytes on immobilized ECM proteins. A clear reduction of PE binding activity on Laminin was observed in the presence of ProtE/5 mAb.

FIG. 5: PE-PilA peptide map used for HDX-MS epitope mapping. 41 pepsin peptides which (taking into account any overlapping regions which are highlighted) corresponds to 96% of the PE-PilA sequence which was considered for HDX-MS analysis.

FIG. 6: Difference in deuterium incorporation generated from the antigen (PE-PilA) alone or bound to the PE/5 mAb. Peptides 22-30 and 122-135 showed a significant difference in deuterium uptake in presence of the mAb.

FIG. 7: Mapping of PE-PilA peptides 22-30 and 122-135 on the 3D structure of the PE-PilA fusion protein LVL-735. Peptides 22-30 and 122-135 (i.e. the binding region of the ProtE/5 mAb) are surface exposed and structurally close, confirming the presence of a conformational epitope. Peptide residues 22-30 and 122-135 are numbered according to the LVL-735 fusion protein of SEQ ID NO: 122. Residues 22-30 and 122-135 also correspond to I41 to Y49 and Y141 to A154 of Protein E of SEQ ID NO: 1.

FIG. 8: Different in deuterium incorporation generated from the antigen (PE-PilA) alone or bound to the PE-PilA/3 mAb. Peptide 166-185 showed a significant difference in deuterium uptake in presence of the mAb.

FIG. 9: Mapping of PE-PilA peptides 166-185 on the 3D structure of the PE-PilA fusion protein LVL-735). Peptides 166-185 (i.e. C62 to A81 of PilA of SEQ ID NO: 56) of the PE-PilA fusion protein LVL-735 (SEQ ID NO: 122) are surface exposed.

FIG. 10: Graphical representation of PE-PilA antigenicity decrease in PE-PilA drug substance thermally stressed at +60° C. Lines and data points correspond to raw data shown in Table 3. Eight samples of PE-PilA fusion protein LVL-735 were selected to be thermally stressed.

FIG. 11: Antigenic activity of PE-PilA measured by IVRP ELISA after exposure of PE-PilA Drug Substance to thermal stress. PE-PilA drug substance (ENHPGPA009) was thermally stressed at +60° C. for up to 6-hours in different containers.

FIG. 12: Antigenic activity of PE-PilA measured by IVRP ELISA after exposure of PE-PilA Drug Substance to long-term thermal stress. PE-PilA drug substance (ENHPGPA009) was thermally stressed at +50° C. for up 7 days (RP=Relative potency).

### DETAILED DESCRIPTION

Terminology

To facilitate review of the various embodiments of this disclosure, the following explanations of terms are provided. Additional terms and explanations are provided in the context of this disclosure.

Unless otherwise explained or defined herein, all technical and scientific terms used herein have the same meaning as commonly understood by one of ordinary skill in the art to which this disclosure belongs. For example, definitions of common terms in molecular biology can be found in Benjamin Lewin, Genes V, published by Oxford University Press, 1994 (ISBN 0-19-854287-9); Kendrew et al. (eds.), The Encyclopaedia of Molecular Biology, published by Blackwell Science Ltd., 1994 (ISBN 0-632-02182-9); and Robert A. Meyers (ed.), Molecular Biology and Biotechnology: A Comprehensive Desk Reference, published by VCH Publishers, Inc., 1995 (ISBN 1-56081-569-8).

All references or patent applications cited within this patent specification are incorporated by reference herein.

Amino acids refers to an amino acid selected from the group consisting of alanine (ala, A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), valine (val, V).

Although methods and materials similar or equivalent to those described herein can be used in the practice or testing of this disclosure, suitable methods and materials are described below.

The abbreviation, “e.g.” is derived from the Latin exempli gratia, and is used herein to indicate a non-limiting example. Thus, the abbreviation “e.g.” is synonymous with the term “for example.”

As used herein, the term “epitope” refers to the portion of a macromolecule (antigen) which is specifically recognised by a component of the immune system e.g. an antibody or a T-cell antigen receptor. The term epitope may refer to that portion of the antigen that makes contact with a particular binding domain of the antigen binding protein. An epitope may be linear or conformational/discontinuous (see definition below). Particular residues comprised within an epitope can be determined through computer modelling programs or via three-dimensional structures obtained through methods known in the art, such as X-ray crystallography. An epitope may reside within the consensus sequence of the invention.

As used herein, the term “conformational epitope (or discontinuous epitope)” refers to an epitope which comprises amino acid residues that are separated by other sequences, i.e. not in a continuous sequence in the antigen's primary sequence. Although the residues may be from different regions of the peptide chain, they are in close proximity in the three-dimensional structure of the antigen. In the case of multimeric antigens, a conformational or discontinuous epitope may include residues from different peptide chains. A conformational epitope may be formed when amino acid residues (to which antibodies can bind) are formed as a result of the polypeptides three-dimensional conformation. The amino acids are located at distinct sites along the linear length of a polypeptide but are co-localised in the 3-D crystal structure. Identifying conformational epitopes can be achieved by numerous methods known in the art. For example, conformational epitopes can be identified using epitope mapping techniques such as Hydrogen Deuterium Exchange Mass Spectrometry (HDX-MS). Instances where a single monoclonal antibody binds to two or more distinct areas of the same linear chain suggest the presence of a conformational epitope. This can be further analysed by mapping said distinct areas onto the 3D crystal structure of a polypeptide molecule. Close structural localisation of the distinct epitopes confirms the presence of a conformational epitope. Conformational epitopes might be preferred for applications involving protein targets in their native state, such as therapeutic applications or flow cytometry. On the other hand, linear epitopes might be preferred for applications in which the protein target is wholly or partially denatured during the sample preparation prior to the immuno assay, such as in Western blot (WB), immunohistochemistry (IHC) or immunofluorescence-based confocal microscopy [Forsstrom et al 2015, PloS One 10(3) e0121673].

A “subject” as used herein is a mammal, including humans, non-human primates, and non-primate mammals. In one aspect, a subject is a human.

As used herein, “immune response” means the sequence of events occurring at the molecular, cellular or tissue level (i.e. at any level of biological organisation) in response to an antigen. In the context of the present disclosure, “immune response” may be the sequence of cellular (cell mediated) and/or humoral (antibody mediated) events occurring in response to an antigen (e.g. antigens on the surface of bacteria, viruses, fungi etc. or in response to antigens presented in the form of an immunogenic fragment, immunogenic composition or vaccine).

As used herein, “immunogenicity” means the ability of an antigen to elicit an immune response.

As used herein, “adjuvant” means a compound or substance (or combination of compounds or substances) that, when administered to a subject in conjunction with an antigen or antigens, for example as part of an immunogenic composition or vaccine, increases or enhances the subject's immune response to the administered antigen or antigens (compared to the immune response obtained in the absence of adjuvant).

As used herein the term “protect or treat” in the context of infection, diseases or conditions caused by H. influenzae means either to protect via prophylaxis or treat via administration post-infection any H. influenzae causing symptom, effect or phenotype. Protection and treatment of an infection, disease or condition caused by H. influenzae includes amelioration of H. influenzae related effects. Treatment or prevention may for example relate to a reduction in the incidence of an infection, disease or condition caused by H. influenzae or a reduction in the number of hospitalizations required as a result of an infection, disease or condition caused by H. influenzae. For the purposes of this invention, “treatment or prevention of exacerbations of COPD” or “or prevention of AECOPD” refers to a reduction in incidence or rate of COPD exacerbations (for instance a reduction in rate of 0.1, 0.5, 1, 2, 5, 10, 20% or more) or a reduction in severity of COPD exacerbations (e.g. airflow obstruction, chronic bronchitis, bronchiolitis or small airways disease and emphysema), for instance within a patient treatment group immunized with the antigen binding proteins, immunogenic compositions or vaccines of the invention.

As used herein, the term “effective amount” in the context of administering a therapy (e.g. an immunogenic composition or vaccine of the invention) to a subject refers to the amount of a therapy which has a prophylactic and/or therapeutic effect(s). In certain embodiments, an “effective amount” refers to the amount of a therapy which is sufficient to achieve one, two, three, four, or more of the following effects: (i) reduce or ameliorate the severity of a bacterial infection or symptom associated therewith; (ii) reduce the duration of a bacterial infection or symptom associated therewith; (iii) prevent the progression of a bacterial infection or symptom associated therewith; (iv) cause regression of a bacterial infection or symptom associated therewith; (v) prevent the development or onset of a bacterial infection, or symptom associated therewith; (vi) prevent the recurrence of a bacterial infection or symptom associated therewith; (vii) reduce organ failure associated with a bacterial infection; (viii) reduce hospitalization of a subject having a bacterial infection; (ix) reduce hospitalization length of a subject having a bacterial infection; (x) increase the survival of a subject with a bacterial infection; (xi) eliminate a bacterial infection in a subject; (xii) inhibit or reduce a bacterial replication in a subject; and/or (xiii) enhance or improve the prophylactic or therapeutic effect(s) of another therapy.

As used herein the term “amino acid modification” relates to any modification which alters the amino acid sequence of a polypeptide. Modifications may include (but is not limited to) polymorphisms, DNA mutations (including single nucleotide polymorphisms), post-translational modifications etc. Modifications include additions/insertions, deletions, point mutations, substitutions etc. Amino acid substitutions may be conservative or non-conservative. In some embodiments, amino acid substitution is conservative. Substitutions, deletions, additions or any combination thereof may be combined in a single variant so long as the variant is an immunogenic polypeptide. Modifications to the amino acid sequence of a polypeptide may be introduced to the DNA, RNA or protein.

As used herein, the term “conservative amino acid substitution” involves substitution of a native amino acid residue with a non-native residue such that there is little or no effect on the size, polarity, charge, hydrophobicity, or hydrophilicity of the amino acid residue at that position, and without resulting in decreased immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics like those of a parental polypeptide.

Embodiments herein relating to “vaccine compositions” of the invention are also applicable to embodiments relating to “immunogenic compositions” of the invention, and vice versa.

As used herein, the term “deletion” is the removal of one or more amino acid residues from the protein sequence. Typically, no more than about from 1 to 6 residues (e.g. 1 to 4 residues) are deleted at any one site within the protein molecule.

As used herein, the term “insertion” is the addition of one or more non-native amino acid residues in the protein sequence. Typically, no more than about from 1 to 10 residues, (e.g. 1 to 7 residues, 1 to 6 residues, or 1 to 4 residues) are inserted at any one site within the protein molecule.

As used herein “signal peptide” refers to a short (less than 60 amino acids, for example, 3 to 60 amino acids) polypeptide present on precursor proteins (typically at the N terminus), and which is typically absent from the mature protein. The signal peptide (sp) is typically rich in hydrophobic amino acids. The signal peptide directs the transport and/or secretion of the translated protein through the membrane. Signal peptides may also be called targeting signals, transit peptides, localization signals, or signal sequences. For example, the signal sequence may be a co-translational or post-translational signal peptide.

As used herein the term “antigen binding protein” refers to antibodies and other protein constructs, such as domains, which are capable of binding to an antigen (for example Protein E or PilA). As used herein the term “PE antigen binding protein of the invention” refers to an an antigen binding protein which binds to Protein E at one or more of amino acid residues within I41 to Y49 (e.g. SEQ ID NO: 133) and Y141 to A154 (e.g. SEQ ID NO: 134) of Protein E or any embodiments thereof. As used herein the term “PilA antigen binding protein of the invention” refers to an antigen binding protein which binds to PilA at one or more of amino acid residues within C62 to A81 (e.g. SEQ ID NO: 135) of PilA or any embodiments thereof.

As used herein the term “antibody” is used in the broadest sense to refer to molecules with an immunoglobulin-like domain (for example IgG, IgM, IgA, IgD or IgE) and includes monoclonal (mAb), recombinant, polyclonal (pAB), chimeric, human, humanised, multispecific antibodies, including bispecific antibodies, and heteroconjugate antibodies; a single variable domain (e.g., VH, VHH, VL, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing (for a summary of alternative “antibody” formats see [Holliger P, Hudson P J. Engineered antibody fragments and the rise of single domains. Nat Biotechnol. 2005; 23(9):1126-36]). Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer or an EGF domain. In one aspect the antibody is a monoclonal antibody (mAB).

As used herein the term “potency” relates to a measure of biological activity using a suitably quantitative biological assay (also called a potency assay or bioassay), based on the attribute of the product which is linked to the relevant biological properties. A relevant, validated potency assay should be part of the specifications for a biotechnological or biological drug substance and/or drug product. Potency is thus the ability of a biologic to exert its desired effect in patients. It will be acknowledged by those of skill in the art however that “potency” in terms of a vaccine potency assay may be a measure which estimates/predicts whether the biologic will elicit the desired effect in patients and such an assay may be used in releasing a vaccine lot to the market. As such “potency” is a relative term, since potency may be determined by reference to a reference standard or an internal standard. The goal of measuring potency in a release assay format is to ensure lot-to-lot (otherwise termed batch-to-batch) consistency.

Identity between polypeptides may be calculated by various algorithms. For example, the Needle program, from the EMBOSS package (Free software; EMBOSS: The European Molecular Biology Open Software Suite (2000). Trends in Genetics 16(6): 276-277) and the Gap program from the GCG® package (Accelrys Inc.) may be used. This Gap program is an implementation of the Needleman-Wunsch algorithm described in: [Needleman, S. B. and Wunsch, C. D. (1970) J. Mol. Biol. 48, 443-453]. The BLOSUM62 scoring matrix can be used, and the gap open and extension penalties were respectively 8 and 2. Identity between two polypeptides is calculated across the entire length of both sequences and is expressed as a percentage of the reference sequence.

Protein E

As used herein “Protein E”, “protein E”, “Prot E”, and “PE” mean Protein E from H. influenzae. Protein E may consist of or comprise the amino acid sequence of SEQ ID NO. 1 (corresponding to SEQ ID NO. 4 of WO2012/139225A1) as well as sequences with at least or exactly 75%, 77%, 80%, 85%, 90%, 95%, 97%, 99% or 100% identity, over the entire length, to SEQ ID NO. 1. In an aspect of the invention, the immunogenic composition comprises Protein E or an immunogenic fragment thereof, suitably an isolated immunogenic polypeptide with at least 70%, 80%, 85%, 90%, 91%, 92%, 93%, 94%, 95%, 96%, 97%, 98%, 99%, or 100% to SEQ ID NO. 1.

(SEQ ID NO: 1)
MKKIILTLSL GLLTACSAQI QKAEQNDVKL APPTDVRSGY
IRLVKNVNYY IDSESIWVDN QEPQIVHFDA VVNLDKGLYV
YPEPKRYARS VRQYKILNCA NYHLTQVRTD FYDEFWGQGL
RAAPKKQKKH TLSLTPDTTL YNAAQIICAN YGEAFSVDKK

Comparison of 53 sequences of Protein E from Haemophilus influenzae (SEQ ID NO. 2-SEQ ID NO. 54; corresponding to SEQ ID Nos. 5 to 57 of WO2012/139225 A1) demonstrated approximately 77% to approximately 100% identity to Protein E as set forth in SEQ ID NO. 1. For example, in the amino acid sequence of Protein E, amino acid #20 may be isoleucine (I) or threonine (T); amino acid #23 may be alanine (A) or valine (V); amino acid #24 may be lysine (K) or glutamic acid (E); amino acid #31 may be alanine (A) or threonine (T); amino acid #32 may be proline (P) or alanine (A); amino acid #34 may be threonine (T) or alanine (A); amino acid #37 may be arginine (R) or glutamine (Q); amino acid #47 may be valine (V) or alanine (A); amino acid #57 may be tryptophan (W) or may be absent (-); amino acid #70 may be alanine (A) or threonine (T); amino acid #93 may be glutamine (Q) or absent (-); amino acid #109 may be threonine (T) or isoleucine (I); amino acid #119 may be glycine (G) or serine (S); amino acid #153 may be glutamic acid (E) or lysine (K); amino acid #156 may be serine (S) or leucine (L); amino acid #160 may be lysine (K) or asparagine (N); amino acid #161 may be lysine (K), isoleucine (I) or absent (-); amino acids #162-#195 may be absent, or as set forth in SEQ ID NO. 15 (with (-) indicating amino acid #166 is absent) or as set forth in SEQ ID NO. 16; or any combination thereof.

Protein E may consist of or comprise an amino acid sequence that differs from SEQ ID NO. 1 at any one or more amino acid selected from the group consisting of: amino acid #20, amino acid #23, amino acid #24, amino acid #31, amino acid #32, amino acid #34, amino acid #37, amino acid #47, amino acid #57, amino acid #70, amino acid #93, amino acid #109, amino acid #119, amino acid #153, amino acid #156, amino acid #160, amino acid #161 and amino acids #162-#195, wherein amino acid #20 is threonine (T); amino acid #23 is valine (V); amino acid #24 is lysine (K); amino acid #31 is threonine (T); amino acid #32 is alanine (A); amino acid #34 is alanine (A); amino acid #37 is glutamine (Q); amino acid #47 is alanine (A); amino acid #57 is absent (-); amino acid #70 is threonine (T); amino acid #93 is absent (-); amino acid #109 is isoleucine (I); amino acid #119 is serine (S); amino acid #153 is lysine (K); amino acid #156 is leucine (L); amino acid #160 is asparagine (N); amino acid #161 is lysine (K) or isoleucine (I); or amino acids #162-#195 are as set forth in SEQ ID NO. 15 (with (-) indicating amino acid #166 is absent) or as set forth in SEQ ID NO. 16.

Protein E may be Protein E from H. influenzae strain 3224A, RdKW20, 86-028NP, R2846, R2866, 3655, PittAA, PittEE, PittHH, PittII, R3021, 22.4-21, 3219C, 3185, 3241A, 038144S1, 810956, 821246, 840645, 902550Z19, A840177, A860514, A950014, 306543X4, A930105, 901905U, A920030, 3221B, 27W116791N, N218, N163, N162, N107, N91, D211PG, D211PD, D201PG, D201PD, D198PG, D198PD, D195PD, D189PG, D189PD, D129CG, D124PG, D124PD, D58PG, D330D, BS433, BS432, 1714, 1128 or BS430. Protein E may be Protein E as set forth in any of SEQ ID NO. 2-SEQ ID NO. 54.

Protein E may be a sequence with at least 95% identity, over the entire length, to SEQ ID NO. 1. Protein E may be a sequence with at least 95% identity, over the entire length, to any of SEQ ID NO. 2-SEQ ID NO. 54.

Immunogenic fragments of Protein E comprise immunogenic fragments of at least 7, 10, 15, 20, 25, 30 or 50 contiguous amino acids of SEQ ID NO. 1. The immunogenic fragments may elicit antibodies which can bind SEQ ID NO. 1. Immunogenic fragments of Protein E comprise immunogenic fragments of at least 7, 10, 15, 20, 25, 30 or 50 contiguous amino acids of SEQ ID NO. 2-SEQ ID NO. 54. The immunogenic fragments may elicit antibodies which can bind the full-length sequence from which the fragment is derived.

For example, there is provided an immunogenic composition comprising an immunogenic fragment of Protein E, suitably an isolated immunogenic polypeptide with at least 70%, 80%, 85%, 90%, 91%, 92%, 93%, 94%, 95%, 96%, 97%, 98%, 99%, or 100% identity to SEQ ID NO. 55 (corresponding to SEQ ID NO: 125 of WO2012/139225A1):

(SEQ ID NO: 55 - Amino acids 20-160 of Protein E)
I QKAEQNDVKL APPTDVRSGY IRLVKNVNYY IDSESIVVVDN
QEPQIVHFDA VVNLDKGLYV YPEPKRYARS VRQYKILNCA
NYHLTQVRTD FYDEFWGQGL RAAPKKQKKH TLSLTPDTTL
YNAAQIICAN YGEAFSVDKK

PilA

As used herein “PilA”, “Pil A” means Pilin A from H. influenzae. PilA may consist of or comprise the protein sequence of SEQ ID NO. 56 (corresponding to SEQ ID NO. 58 from WO2012/139225A1) (MKLTTQQTLK KGFTLIELMI VIAIIAILAT IAIPSYQNYT KKAAVSELLQ ASAPYKADVE LCVYSTNETT NCTGGKNGIA ADITTAKGYV KSVTTSNGAI TVKGDGTLAN MEYILQATGN AATGVTWTTT CKGTDASLFP ANFCGSVTQ) as well as sequences with 80% to 100% identity to SEQ ID NO. 56. For example, PilA may be at least 80%, 85%, 90%, 95%, 97% or 100% identical to SEQ ID NO. 58. Full length comparison of 64 sequences of PilA from Haemophilus influenzae (SEQ ID NO. 56-SEQ ID NO. 119, corresponding to SEQ ID NO. 58-SEQ ID NO. 121 of WO20012/139225A1) demonstrated approximately 80% to 100% identity to PilA as set forth in SEQ ID NO. 56.

For example, in the amino acid sequence of PilA, amino acid #6 may be glutamine (Q) or leucine (L); amino acid #7 may be glutamine (Q) or threonine (T); amino acid #37 may be glutamine (Q) or lysine (K); amino acid #44 may be alanine (A) or serine (S); amino acid #57 may be alanine (A) or serine (S); amino acid #67 may be asparagine (N) or glycine (G); amino acid #68 may be glutamic acid (E) or lysine (K); amino acid #69 may be threonine (T) or proline (P); amino acid #71 may be lysine (K), asparagine (N), serine (S) or threonine (T); amino acid #73 may be threonine (T), serine (S) or methionine (M); amino acid #76 may be lysine (K), serine (S) or asparagine (N); amino acid #84 may be threonine (T) or lysine (K); amino acid #86 may be alanine (A) or valine (V); amino acid #91 may be lysine (K) or alanine (A); amino acid #94 may be threonine (T), isoleucine (I) or lysine (K); amino acid #96 may be serine (S) or glutamine (Q); amino acid #97 may be asparagine (N) or serine (S); amino acid #99 may be alanine (A) or glycine (G); amino acid #103 may be alanine (A) or lysine (K); amino acid #109 may be aspartic acid (D), alanine (A) or threonine (T); amino acid #110 may be glycine (G), asparagine (N), or arginine (R); amino acid #112 may be serine (S) or glutamic acid (E); amino acid #114 may be threonine (T) or isoleucine (I); amino acid #116 may be threonine (T) or glutamine (Q); amino acid #118 may be glutamic acid (E), threonine (T), alanine (A), lysine (K) or serine (S); amino acid #121 may be serine (S) or alanine (A); amino acid #122 may be alanine (A) or threonine (T); amino acid #123 may be lysine (K), threonine (T) or alanine (A); amino acid #128 may be lysine (K) or threonine (T); amino acid #135 may be aspartic acid (D) or glutamic acid (E); amino acid #136 may be alanine (A) or threonine (T); amino acid #145 may be glycine (G) or arginine (R); amino acid #149 may be glutamine (Q) or lysine (K); or any combination thereof.

PilA may consist of or comprise an amino acid sequence that differs from SEQ ID NO. 56 at any or more amino acid selected from the group consisting of amino acid #6, amino acid #7, amino acid #37, amino acid #44, amino acid #57, amino acid #67, amino acid #68, amino acid #69, amino acid #71, amino acid #73, amino acid #76, amino acid #84, amino acid #86, amino acid #91, amino acid #94, amino acid #96, amino acid #97, amino acid #99, amino acid #103, amino acid #109, amino acid #110, amino acid #112, amino acid #114, amino acid #116, amino acid #118 amino acid, #121, amino acid #122, amino acid #123, amino acid #128, amino acid #135, amino acid #136, amino acid #145 and amino acid #149, wherein amino acid #6 is leucine (L); amino acid #7 is threonine (T); amino acid #37 is lysine (K); amino acid #44 is serine (5); amino acid #57 is serine (5); amino acid #67 is glycine (G); amino acid #68 is lysine (K); amino acid #69 is proline (P); amino acid #71 is lysine (K), serine (S) or threonine (T); amino acid #73 is serine (S) or methionine (M); amino acid #76 is serine (S) or asparagine (N); amino acid #84 is lysine (K); amino acid #86 is valine (V); amino acid #91 is alanine (A); amino acid #94 is isoleucine (I) or lysine (K); amino acid #96 is glutamine (Q); amino acid #97 is serine (5); amino acid #99 is glycine (G); amino acid #103 is alanine (A); amino acid #109 is aspartic acid (D) or threonine (T); amino acid #110 is glycine (G) or arginine (R); amino acid #112 is serine (5); amino acid #114 is threonine (T); amino acid #116 is threonine (T); amino acid #118 is glutamic acid (E), alanine (A), lysine (K) or serine (5); amino acid #121 is serine (5); amino acid #122 is threonine (T); amino acid #123 is lysine (K) or alanine (A); amino acid #128 is lysine (K); amino acid #135 is glutamic acid (E); amino acid #136 is threonine (T); amino acid #145 is arginine (R); amino acid #149 is lysine (K).

PilA may be PilA from H. influenzae strain NTHi3219C, NTHi3224A, NTHi12, NTHi44, NTHi67, 1054MEE, 1729MEE, 1728MEE, 1885MEE, 1060MEE, RdKW20, 214NP, 1236MEE, 1714MEE, 1128MEE, 86-028NP, R2846, R2866, 3655, PittAA, PittGG, PittII, R3021, 22.4-21, 3185A, 3221B, 3241A, 038144S1, 821246, 840645, 902550Z19, A840177, A920030, A950014, 901905U, A920029, A930105, 306543X4, N218, N163, N162, N120, N107, N92, N91, D219PG, D211PG, D211PD, D204CD, D198PG, D198PD, D195PD, D195CD, D189PG, D189PD, D124PG, D124PD, D124CG, D58PG, BS433, BS432, BS430, 1714 or 1128. An amino acid sequence for PilA from H. influenzae strain D204CD is set forth in SEQ ID NO. 104, wherein X at position #116 is either glutamine (Q) or leucine (L); ambiguity as to the amino acid at position #116 could be cleared up by technical resolution of the second nucleotide encoding amino acid #116, clarifying the PilA sequence for strain D204CD. PilA may be PilA as set forth in any of SEQ ID NO. 56-SEQ ID NO. 119.

PilA may be a sequence with at least 95% identity, over the entire length, to any of SEQ ID NO. 56-SEQ ID NO. 119.

Immunogenic fragments of PilA comprise immunogenic fragments of at least 7, 10, 15, 20, 25, 30 or 50 contiguous amino acids of SEQ ID NO. 56-SEQ ID NO. 119. The immunogenic fragments may elicit antibodies which can bind the full-length sequence from which the fragment is derived. For example, immunogenic fragments of PilA comprise immunogenic fragments of at least 7, 10, 15, 20, 25, 30 or 50 contiguous amino acids of SEQ ID NO. 56. The immunogenic fragments may elicit antibodies which can bind SEQ ID NO. 56.

In another aspect of the invention, the immunogenic composition comprises an immunogenic fragment of PilA, suitably an isolated immunogenic polypeptide with at least 70%, 80%, 85%, 90%, 91%, 92%, 93%, 94%, 95%, 96%, 97%, 98%, 99%, or 100% to SEQ ID NO: 56 or SEQ ID NO: 57-119. For example, immunogenic fragments of PilA comprise immunogenic fragments of at least 7, 10, 15, 20, 25, 30 or 50 contiguous amino acids of SEQ ID NO. 56. The immunogenic fragments may elicit antibodies which can bind SEQ ID NO. 56.

In another aspect of the invention, the immunogenic composition comprises an immunogenic fragment of PilA, suitably an isolated immunogenic polypeptide with at least 70%, 80%, 85%, 90%, 91%, 92%, 93%, 94%, 95%, 96%, 97%, 98%, 99%, or 100% to SEQ ID NO. 120 (corresponding to SEQ ID NO: 127 of WO2012/139225A1):

SEQ ID NO. 120: Amino acids 40-149 of PilA from
H. influenzae strain 86-028NP
T KKAAVSELLQ ASAPYKADVE LCVYSTNETT NCTGGKNGIA
ADITTAKGYV KSVTTSNGAI TVKGDGTLAN MEYILQATGN
AATGVTVVTTT CKGTDASLFP ANFCGSVTQ.

PEPilA Fusion Protein

A PE-PilA fusion protein comprises an immunogenic fragment of Protein E and an immunogenic fragment of PilA in the form of a fusion protein (PE-PilA). Suitable fusions are disclosed in WO2012/139225A1) and a preferred fusion is LVL-735 of SEQ ID NO:121 (corresponding to SEQ ID NO: 194 of WO2012/139225 A1). In particular embodiment of the invention, the signal peptide has been removed as demonstrated in SEQ ID NO. 122 (corresponding to SEQ ID No. 219 of WO2012/139225A1).

Thus, in particular embodiments of the invention, the immunogenic composition comprises both Protein E and PilA in the form of a fusion protein, suitably an isolated immunogenic polypeptide with at least 70%, 80%, 85%, 90%, 91%, 92%, 93%, 94%, 95%, 96%, 97%, 98%, 99%, or 100% to LVL-735, with the signal peptide, SEQ ID NO. 121 (Corresponding to SEQ ID No. 194 of WO2012/139225A1).

SEQ ID NO. 121: LVL735 (protein):
(pelB sp)(ProtE aa 20-160)(GG)(PilA aa40-149):
MKYLLPTAAA GLLLLAAQPA MAIQKAEQND VKLAPPTDVR
SGYIRLVKNV NYYIDSESIW VDNQEPQIVH FDAVVNLDKG
LYVYPEPKRY ARSVRQYKIL NCANYHLTQV RTDFYDEFWG
QGLRAAPKKQ KKHTLSLTPD TTLYNAAQII CANYGEAFSV
DKKGGTKKAA VSELLQASAP YKADVELCVY STNETTNCTG
GKNGIAADIT TAKGYVKSVT TSNGAITVKG DGTLANMEYI
LQATGNAATG VTWTTTCKGT DASLFPANFC GSVTQ

Thus, in particular embodiments of the invention, the immunogenic composition comprises both Protein E and PilA in the form of a fusion protein, suitably an isolated immunogenic polypeptide with at least 70%, 80%, 85%, 90%, 91%, 92%, 93%, 94%, 95%, 96%, 97%, 98%, 99%, or 100% to LVL-735, with the signal peptide, SEQ ID NO. 122 (Corresponding to SEQ ID No. 219 of WO2012/139225A1).

SEQ ID NO. 122: PE-PilA fusion protein without
signal peptide
IQKAEQND VKLAPPTDVR SGYIRLVKNV NYYIDSESIW
VDNQEPQIVH FDAVVNLDKG LYVYPEPKRY ARSVRQYKIL
NCANYHLTQV RTDFYDEFWG QGLRAAPKKQ KKHTLSLTPD
TTLYNAAQII CANYGEAFSV DKKGGTKKAA VSELLQASAP
YKADVELCVY STNETTNCTG GKNGIAADIT TAKGYVKSVT
TSNGAITVKG DGTLANMEYI LQATGNAATG VTWTTTCKGT
DASLFPANFC GSVTQ

The immunogenicity of Protein E (PE) and Pilin A (PilA) polypeptides may be measured as described in WO2012/139225A1. Essentially, the immune response directed against PE and PilA (or a PE-PilA fusion protein) can be evaluated in vivo, for example in Balb/c mice (although other pre-clinical species could be used). Animals are immunized, for example by the intramuscular route with PE, PilA (or a PE-PilA fusion protein) with and without a suitable adjuvant. A control group can be vaccinated with adjuvant alone. Antibody response directed against each antigen can be determined in individual sera by measuring IgG antibody titers using ELISA. Serum Bactericidal Assays can also be performed. All such assays to test the immunogenicity of antigen(s) are within the realm of the person skilled in the art.

Antigen Binding Proteins

Protein E Antigen Binding Protein

The present invention provides an antigen binding protein which binds Protein E (“PE antigen binding protein”). Unless otherwise stated, amino acid numbering in relation to Protein E is in respect of Protein E of SEQ ID NO: 1.

In particular, the present invention provides an antigen binding protein which binds to Protein E at one or more of amino acid residues within I41 to Y49 (e.g. SEQ ID NO: 133) and Y141 to A154 (e.g. SEQ ID NO: 134) of Protein E. Reference to SEQ ID NO: 133 and SEQ ID NO: 134 are exemplar only thus, in an embodiment the PE antigen binding protein of the invention binds to Protein E at one or more of amino acid residues within I41 to Y49 and Y141 to A154 of Protein E wherein said regions differ by 1, 2, 3, 4 or 5 amino acid modifications to SEQ ID NO: 133 or SEQ ID NO: 134.

In an embodiment, the PE antigen binding protein of the invention binds to Protein E at one or more of amino acid residues within I41 to Y49 of Protein E (e.g. SEQ ID NO: 133) and at one or more of amino acid residues within Y141 to A154 of Protein E (e.g. SEQ ID NO: 134). In an embodiment, the PE antigen binding protein of the invention binds to an epitope within or comprising amino acid residues I41 to Y49 and Y141 to A154 of Protein E (e.g. the amino acid residue of SEQ ID NO: 133 and SEQ ID NO: 134).

In an embodiment, the PE antigen binding protein of the invention binds to an epitope comprising or consisting of amino acid residues 141 to Y49 (e.g. SEQ ID NO: 133) and Y141 to A154 (e.g. SEQ ID NO: 134) of Protein E. In an embodiment, the PE antigen binding protein of the invention binds to an epitope consisting of amino acid residues I41 to Y49 (e.g. SEQ ID NO: 133) and Y141 to A154 (e.g. SEQ ID NO: 134) of Protein E. Reference to amino acid residues I41 to Y49 and Y141 to A154 of Protein E relate to the Protein E sequence as defined in SEQ ID NO:1.

Amino acid residue ranges referred to herein (e.g. I41 to Y49 and Y141 to A154) includes the “end” amino acid residues I41 and Y49 and Y141 and A154 as well as any (or all) residues within said ranges. In an embodiment the PE antigen binding protein of the invention may bind to any residues within regions I41 to Y49 and Y141 to A154 of Protein E.

In an embodiment the PE antigen binding protein of the invention binds to an epitope comprising or consisting of i) SEQ ID NO: 133 and SEQ ID NO: 134 or ii) variants of SEQ ID NO: 133 and 134, wherein said variants comprise 1, 2 or 3 amino acid modifications. Said amino acid modifications are single amino acid modifications, i.e. 1 single amino acid modification, 2 single acid modifications or 3 single amino acid modifications. SEQ ID NO: 133 and SEQ ID NO: 134 correspond to amino acid residues I41 to Y49 [SEQ ID NO: 133] and Y141 to A154 [SEQ ID NO: 134] of Protein E.

In a further embodiment the PE antigen binding protein of the invention is capable of binding to Protein E when Protein E is present as a fragment or fusion protein. For example, the PE antigen binding protein of the invention may bind to the PE-PilA fusion protein of SEQ ID NO: 121 (LVL-735 including signal peptide) and SEQ ID NO: 122 (LVL-735 minus signal peptide) or sequences with at least 80% identity to SEQ ID NO: 121 or SEQ ID NO: 122. In an embodiment the PE antigen binding protein of the invention binds to Protein E at one or more of amino acid residues within the regions I22 to Y30 and Y122 to A135 of the PE-PilA fusion protein of SEQ ID NO: 122 (i.e. LVL-735 fusion protein lacking signal peptide) or a sequence with at least 70%, at least 75%, at least 80%, at least 85%, at least 90%, at least 95% or at least 100% identity to SEQ ID NO: 122.

It will be understood by a person skilled in the art, that reference to amino acid residues within I41 to Y49 and Y141 to A154 of Protein E is referring to the full length Protein E as defined in SEQ ID NO:1. Furthermore, reference to amino acid residues within I41 to Y49 and Y141 to A154 of Protein E is referring to the amino acid number counting consecutively from the N-terminus of the amino acid sequence, of SEQ ID NO. 1. Amino acid residues within I41 to Y49 and Y141 to A154 refers to the amino acids from the 41^{st} and 49^{th} along with the 141^{st} to 154^{th} amino acid of SEQ ID NO. 1.

As used herein, the amino acid residues within I41 to Y49 of Protein E refers to the amino acid numbers of SEQ ID NO: 1 i.e. IRLVKNVNY (SEQ ID NO: 133). As used herein, the amino acid residues within Y141 to A154 of Protein E refers to the amino acid numbers of SEQ ID NO: 1 i.e. YNAAQIICANYGEA (SEQ ID NO: 134)

A person skilled in the art will understand that when the Protein E amino acid sequence is a variant and/or fragment of an amino acid sequence of SEQ ID NO. 1, such as an amino acid sequence at least 80%, 85%, 90%, 92%, 95%, 96%, 97%, 98% or 99% identical to SEQ ID NO. 1, the reference to “I41 to Y49 and Y141 to A154” refers to a the position that would be equivalent to the defined position, if this sequence was lined up with an amino acid sequence of SEQ ID NO. 1 in order to maximise the sequence identity between the two sequences (Sequence alignment tools are not limited to Clustal Omega (www(.)ebi(.)ac(.)ac(.)uk) MUSCLE (www(.)ebi(.)ac(.)uk), or T-coffee (www(.)tcoffee(.)org). In one aspect, the sequence alignment tool used is Clustal Omega (www(.)ebi(.)ac(.)ac(.)uk).

Variants of SEQ ID NO.1 could lead to a difference in the actual amino acid position of the consensus sequence in the sequence, however, by lining the sequence up with the reference sequence, the amino acid in in an equivalent position to the corresponding amino acid in the reference sequence can be identified and hence the appropriate amino acids identified. For example, I41 numbered with respect to SEQ ID NO. 1 encompasses V41 of SEQ ID NO. 21 or SEQ ID NO: 30 (examples only) as well as I41 of SEQ ID NO. 1. The terminology “Numbered with respect to” is used herein to reference a location in an amino acid sequence while not being limited to that referenced amino acid sequence.

In an embodiment the PE antigen binding protein of the invention binds to Protein E at one or more of amino acid residues within the region 41-49 and 141-154 of Protein E (of SEQ ID NO: 1). In an embodiment the PE antigen binding protein of the invention binds to Protein E at one or more of amino acid residues within the region 41-49 and 141-154 of Protein E (of SEQ ID NO: 1) or sequence with at least 70%, at least 80%, at least 90%, at least 95% or 100% identity to SEQ ID NO: 1. In an embodiment the PE antigen binding protein of the invention binds to an epitope within or comprising amino acid residues 41-49 and 141-154 of Protein E (of SEQ ID NO: 1). In an embodiment the PE antigen binding protein of the invention binds to an epitope within or comprising amino acid residues 41-49 and 141-154 of Protein E (of SEQ ID NO: 1) or sequence with at least 70%, at least 80%, at least 90%, at least 95% or 100% identity to SEQ ID NO: 1.

Reference to amino acid residues within I41 to Y49 and Y141 to A154 may also be referring to the corresponding residues within a fragment of Protein E (such as the fragment described in SEQ ID NO: 55). This is only to the extent that the corresponding amino acids of I41 to Y49 and Y141 to A154 remain present in the sequence i.e. wherein said fragment retains the epitope of the invention. For example, within the fragment of Protein E of SEQ ID NO: 55 (corresponding to amino acids 20-160 of SEQ ID NO: 1), the corresponding residues are I22 to Y30 and Y122 to A135.

Reference to amino acid residues within I41 to Y49 and Y141 to A154 may also be referring to those corresponding residues within a fusion protein comprising Protein E (wherein Protein E may be fused to any heterologous polypeptide (for example PilA)). This is only to the extent that the amino acids corresponding to I41 to Y49 and Y141 to A154 remain present in the fusion protein i.e. wherein said fusion retains the epitope of the invention. For example, within the PE-PilA fusion protein of SEQ ID NO: 122 (LVL-735 minus signal peptide), the corresponding residues are I22 to Y30 and Y122 to A135.

In an embodiment, the PE antigen binding protein of the invention, binds to an epitope wherein the epitope is a conformational epitope. In an embodiment the PE antigen binding protein of the invention binds to an epitope within or comprising amino acid residues I41 to Y49 and Y141 to A154 of Protein E wherein said epitope is a conformational epitope. In an embodiment the PE antigen binding protein of the invention binds to a conformational epitope wherein said conformational epitope is formed by the close proximity of amino acid residues I41 to Y49 and Y141 to A154 in the three-dimensional structure of Protein E.

In an embodiment the PE antigen binding protein of the invention binds to an epitope that is associated with an immunogenically active form of Protein E. In an embodiment the PE antigen binding protein of the invention binds to an epitope that is associated with Protein E in a conformation where it is immunogenically active. In an embodiment the PE antigen binding protein of the invention binds to an epitope that is associated with an immunogenically active form of a PE-PilA fusion protein (e.g. LVL-735 of SEQ ID NO: 122). In an embodiment the PE antigen binding protein of the invention binds to an epitope within or comprising amino acid residues I41 to Y49 and Y141 to A154 of Protein E wherein said epitope is associated with an immunogenically active form of Protein E. In an embodiment the PE antigen binding protein of the invention binds (or preferentially binds) to a Protein E antigen which is capable of eliciting an immune response in a mammal, preferably in a human being. In an embodiment, the PE antigen binding protein of the invention binds (or preferentially binds) to a Protein E antigen which is protective against diseases associated with H. influenzae. In an embodiment, the PE antigen binding protein of the invention binds (or preferentially binds) to a Protein E antigen which is protective against AECOPD.

In an embodiment the PE antigen binding protein of the invention binds to an epitope that is associated with the native conformation of Protein E. In an embodiment the PE antigen binding protein of the invention binds to an epitope within or comprising amino acid residues I41 to Y49 and Y141 to A154 of Protein E wherein said epitope is associated with the native conformation of Protein E. In an embodiment the PE antigen binding protein of the invention binds to Protein E in its native conformation with a higher specificity and/or than to Protein E in a non-native conformation. In an embodiment, the PE antigen binding protein of the invention binds to Protein E in its native conformation with higher affinity than to Protein E in a non-native conformation. In an embodiment, the PE antigen binding protein of the invention binds to Protein E in its native conformation with higher specificity than to Protein E in a non-native conformation

In an embodiment the PE antigen binding protein of the invention is unable to bind to Protein E in its non-native (or significantly non-native) conformation or less PE antigen binding protein of the invention is capable of binding Protein E in its non-native conformation. In an embodiment the PE antigen binding protein of the invention binds to Protein E in its non-native (or significantly non-native) conformation with less specificity and/or affinity than to Protein E in its native conformation. For example, the PE antigen binding protein of the invention binds to Protein E in its native (or substantially native) conformation with higher specificity and/or higher affinity than to a Protein E which is denatured.

In an embodiment Protein E may be denatured or adopt a non-native conformation for example via thermal stress, freeze-thawing, pH alterations, oxidation, enzymatic digestion (e.g. trypsin digestion), mishandling or process errors. In an embodiment Protein E may be denatured or adopt a non-native conformation for example via reduction (e.g. methionine reduction) or via light exposure. In an embodiment Protein E may be denatured via thermal stress. In an embodiment, Protein E may be denatured following exposure to temperatures greater than room temperature (i.e. greater than approximately 20° C. to 22° C.), greater than 30° C., greater than 40° C., greater than 50° C., greater than 60° C. or greater than 70° C. In an embodiment, Protein E may be denatured at 65° C.±5° C. In an embodiment, Protein E may be denatured via thermal stress for up to 1 hour, up to 2 hours, up to 4 hours, up to 6 hours, up to 8 hours, up to 12 hours, up to 24 hours. In an embodiment, Protein E may be denatured via thermal stress for greater than 24 hours.

In an embodiment the PE antigen binding protein of the invention binds to a vaccine sample comprising a Protein E in its native conformation with a higher specificity and/or affinity as compared to a vaccine sample comprising a Protein E immunogen which has lost the relevant epitope. In an embodiment, the Protein E immunogen has lost the relevant epitope for example due to denaturation, aggregation or breakdown during storage or by mishandling. In an embodiment, the Protein E immunogen has lost the epitope within or comprising amino acid residue I41 to Y49 and Y141 to A154 of Protein E due to denaturation, aggregation or breakdown during storage or mishandling.

In an embodiment the PE antigen binding protein of the invention inhibits vitronectin binding. Protein E binds to vitronectin by the binding domain PKRYARSVRQ YKILNCANYH LTQVR (SEQ ID NO: 136 which corresponds to amino acids 84-108 of SEQ ID NO: 1). Vitronectin is an important inhibitor of the terminal complement pathway [J. Immunology 183:2593-2601 (2009)]. In an embodiment the PE antigen binding protein of the invention is capable of inhibiting vitronectin despite binding to an epitope located elsewhere on the protein to the vitronectin binding domain of SEQ ID NO: 1.

In an embodiment the PE antigen binding protein of the invention inhibits laminin binding. Protein E binds to laminin by the laminin binding domain of SEQ ID NO: 137 (i.e. residues 41-68 of SEQ ID NO: 1). Laminin is a major glycoprotein component of basement membranes and functions as an adhesion molecule. Cell attachment to laminin initiates physiological responses such as cell growth and motility, epithelial cell differentiation and leukocyte phagocytosis. As described in [Singh et al 2013 Infection and Immunity 81(3): 801-814], the region comprising Protein E amino acids 41 to 68 of SEQ ID NO: 1 (SEQ ID NO: 137) interacts with laminin, an abundant extracellular matrix protein in the basement membrane, and this interaction leads to better adhesion of NTHI to host tissues. The laminin and vitronectin binding sites on the PE molecule are completely separate and do not interfere with each other during binding. In an embodiment the PE antigen binding protein of the invention inhibits both vitronectin and laminin binding. In an embodiment, the PE antigen binding protein of the invention inhibits only laminin binding. In an embodiment the PE antigen binding protein of the invention competes for binding to Protein E with laminin. In an embodiment the PE antigen binding protein of the invention competes with laminin for binding to Protein E, specifically at the laminin binding site of SEQ ID NO: 137 (i.e. at amino acids 41-68 of SEQ ID NO: 1).

In an embodiment the PE antigen binding protein of the invention inhibits biofilm formation. Inhibition of biofilm formation can be measured for example using the methodology described in Example 2.

In an embodiment the PE antigen binding protein of the invention is an antibody. In an embodiment the PE antigen binding protein (or antibody) of the invention is a monoclonal antibody (mAb), optionally an IgG2a monoclonal antibody, optionally ProtE/5. In an embodiment the isotype of the mAb is a mouse IgG2A. In an embodiment, the mAb is an anti-PE mAb. In an embodiment the mAb is ProtE/5. In an embodiment the antibody of the invention is produced by the Repetitive Immunisation Multiple Sites (RIMMS) method is described in [Eric P. Dixon, Cell Biology (Third Edition) A Laboratory Handbook: Chapter 58—Rapid Development of Monoclonal Antibodies Using Repetitive Immunizations, Multiple Sites. Academic Press. 2006; 1:483-90] which is incorporated herein by reference.

In an embodiment the PE antigen binding protein of the invention comprises: a variable heavy (VH) region comprising a sequence at least 80% identical to the sequence of SEQ ID NO: 124; and/or a variable light (VL) region comprising a sequence at least 80% identical to the sequence of SEQ ID NO: 129. In an embodiment the PE antigen binding protein of the invention comprises: a variable heavy (VH) region comprising a sequence at least 80% identical to the sequence of SEQ ID NO: 124; and a variable light (VL) region comprising a sequence at least 80% identical to the sequence of SEQ ID NO: 129. For example, an antigen binding comprising: a variable heavy (VH) region comprising a sequence at least 80%, at least 85%, at least 90%, at least 91%, at least 92%, at least 93%, at least 94%, at least 95%, at least 96%, at least 97%, at least 98%, at least 99% or 100% identical to the sequence of SEQ ID NO: 124; and a variable light (VL) region comprising a sequence at least 80%, at least 85%, at least 90%, at least 91%, at least 92%, at least 93%, at least 94%, at least 95%, at least 96%, at least 97%, at least 98%, at least 99% or 100% identical to the sequence of SEQ ID NO: 129.

The PE antigen binding protein sequence may be a variant sequence with up to 3 amino acid modifications. For example, the modification is a substitution, addition or deletion. For example, the variant sequence may have up to 3, 2 or 1 amino acid substitution(s), addition(s) and/or deletion(s). The sequence variation may exclude the CDR(s), for example the CDR(s) is intact, and the variation is in the remaining portion of the VH or VL (or HC or LC) sequence, so that the CDR sequence is fixed. The variant sequence substantially retains the biological characteristics of the unmodified antigen binding protein.

As used herein the term “VH Region” or “VL Region” refers to the variable portions of the heavy (VH) and light (VL) chains respectively. These regions form the binding pocket, which binds the specific antigens, and contains the major diversity of the immunoglobulin.

In an embodiment the PE antigen binding protein of the invention comprises: a VH region comprising SEQ ID NO: 124; and/or a VL region comprising SEQ ID NO: 129. In an embodiment the PE antigen binding protein of the invention comprises: a VH region comprising SEQ ID NO: 124; and a VL region comprising SEQ ID NO: 129. In an embodiment the PE antigen binding protein of the invention comprises: a VH region consisting of SEQ ID NO: 124; and/or a VL region consisting of SEQ ID NO: 129. In an embodiment the PE antigen binding protein of the invention comprises: a VH region consisting of SEQ ID NO: 124; and a VL region consisting of SEQ ID NO: 129.

In an embodiment the PE antigen binding protein of the invention comprises a VH region encoded by SEQ ID NO: 123 and/or a VL region encoded by SEQ ID NO: 128.

There is also provided an antigen binding protein comprising any one or a combination of CDRs selected from CDR-H1, CDR-H2, CDR-H3 from SEQ ID NO: 124 , and/or CDR-L1, CDR-L2, CDR-L3 from SEQ ID NO: 125; or (ii) a CDR variant of (i), wherein the variant has 1, 2, or 3 amino acid modifications in each CDR, which is able to bind to Protein E at one or more of amino acid residues within 141 to Y49 and Y141 to A154 of Protein E (e.g. SEQ ID NO: 133 and SEQ ID NO: 134). In an embodiment the PE antigen binding protein of the invention comprises any one ora combination of CDRs selected from CDR-H1 (SEQ ID NO: 125), CDR-H2 (SEQ ID NO: 126) or CDR-H3 (SEQ ID NO: 127), and/or CDR-L1 (SEQ ID NO: 130), CDR-L2 (SEQ ID NO: 131) or CDR-L3 (SEQ ID NO: 132) or (ii) a CDR variant of (i), wherein the variant has 1, 2, or 3 amino acid modifications in each CDR, which is able to bind to Protein E at one or more of amino acid residues within 141 to Y49 and Y141 to A154 of Protein E (e.g. SEQ ID NO: 133 and SEQ ID NO: 134).

The CDR regions may be identified using any method known to those of skill in the art. The CDR regions of the VH chain (of SEQ ID NO: 124) and VL chain (of SEQ ID NO: 129) are shown in Table 1 below.

TABLE 1
CDR Regions of the VH (SEQ ID NO: 124) and VL
(SEQ ID NO: 129) regions of the ProtE/5 mAb
of the invention.

| CDR | SEQ ID NO | Sequence |
|---|---|---|
| CDR-H1 | 125 | NYGMN |
| CDR-H2 | 126 | WINTYTGEPTYADDFKG |
| CDR-H3 | 127 | GGYPSSRAPPY |
| CDR-L1 | 130 | KASQDVGTAVA |
| CDR-L2 | 131 | WASTRHT |
| CDR-L3 | 132 | QQYSSYPFT |

In a further aspect, there is provided an antigen binding protein that binds to Protein E and competes for binding at one or more of amino acid residues within I41 to Y49 (e.g. SEQ ID NO: 133) and Y141 to A154 (e.g. SEQ ID NO: 134) of Protein E with reference to the antigen binding protein with a VH region comprising SEQ ID NO: 124 and a VL region comprising SEQ ID NO: 129. Suitable assays to analyse whether antibodies compete for binding are well known in the art (for example see Kwak & Yoon et al 1996, J Immunol Methods 191(1): 49-54).

The binding of the antibody of the invention to protein Eat one or more of amino acid residues within I41 to Y49 and Y141 to A154 of Protein E, can be determined using Hydrogen-Deuterium exchange coupled with Mass Spectrometry (HDX-MS). Briefly, HDX-MS detects structural changes of a protein due to ligand binding, protein-protein interaction, post-translational modifications and others (the method is described in Example 3). The epitope region on the Protein E which is targeted by mAb ProtE/5 will display reduced exchange rates in the presence of ProtE/5 relative to Protein E alone which can be identified by HDX-MS. Following the exchange, the reaction is quenched with an acidic pH and low temperature. The proteins are digested with pepsin or other acidic proteases and analysed via mass spectrometry.

The present invention also provides a nucleic acid sequence which encodes the antigen binding protein as defined herein.

The present invention also provides an expression vector comprising the nucleic acid sequence as defined herein. The present invention also provides a recombinant host cell comprising the nucleic acid sequence as defined herein, or the expression vector as defined herein.

The present invention also provides a method for the production of the antigen binding protein as defined herein, which method comprises culturing the host cell as defined herein under conditions suitable for expression of said nucleic acid sequence or vector, whereby the antigen binding protein is expressed and purified.

The present invention also provides an antigen binding protein produced by the method described herein.

The present invention also provides a pharmaceutical composition comprising the antigen binding protein as defined herein, and one or a combination of pharmaceutically acceptable carriers, excipients or diluents.

PilA Antigen Binding Protein

The present invention provides an antigen binding protein which binds PilA (“PilA antigen binding protein”). According to a further aspect of the invention, there is provided an antigen binding protein which binds to PilA at one or more of amino acid residues within C62 to A81 (e.g. SEQ ID NO:135) of PilA. Reference to SEQ ID NO: 135 is exemplar only thus, in an embodiment the PilA antigen binding protein of the invention binds to PilA at one or more of amino acid residues within C62 to A81 wherein said regions differ by 1, 2, 3, 4 or 5 amino acid modifications to SEQ ID NO: 135.

In an embodiment, the PilA antigen binding protein of the invention binds to an epitope within or comprising amino acid residues C62 to A81 of PilA (e.g. SEQ ID NO: 135). In an embodiment, the PilA antigen binding protein of the invention binds to an epitope comprising or consisting of amino acid residues C62 to A81 of PilA (e.g. SEQ ID NO: 135). Reference to amino acid residues C62 to A81 of PilA corresponds to the corresponding residues in SEQ ID NO: 56. Amino acid residues C62 to A81 thus corresponds to CVYSTNETTNCTGGKNGIAA (SEQ ID NO: 135).

Amino acid residue ranges referred to (e.g. C62 to A81) includes the “end” amino acid residues C62 and A81 as well as any (or all) residues within said ranges. In an embodiment the PilA antigen binding protein of the invention may bind to any residues within regions C62 to A81.

In an embodiment the PilA antigen binding protein of the invention binds to an epitope comprising or consisting of i) SEQ ID NO: 135 or ii) variants of SEQ ID NO: 135, wherein said variants comprise 1, 2 or 3 amino acid modifications. Said amino acid modifications are single amino acid modifications, i.e. 1 single amino acid modification, 2 single amino acid modifications or 3 single amino acid modifications. In an embodiment, the epitope is a conformational epitope. Said conformational epitope is formed as a result of a disulphide bond formed between the two cysteine residues in SEQ ID NO: 135 (i.e. C62 and C72), specifically between the thiol groups of C62 and C72 (of SEQ ID NO: 135) by oxidative folding.

In a further embodiment the PilA antigen binding protein is capable of binding to PilA when PilA is present as a fragment or fusion protein. For example, the PilA antigen binding protein of the invention may bind to the PE-PilA fusion protein of SEQ ID NO: 121 (LVL-735 including signal peptide) and SEQ ID NO: 122 (LVL-735 minus signal peptide) or sequences with at least 80% similarity to SEQ ID NO: 121 or 122. In an embodiment the antigen binding protein of the invention binds to Protein Eat one or more of amino acid residues within the regions I22 to Y30 and Y122 to A135 of the PE-PilA fusion protein of SEQ ID NO: 122 (i.e. LVL-735 fusion protein lacking signal peptide) or a sequence with at least 70%, at least 75%, at least 80%, at least 85%, at least 90%, at least 95% or at least 100% identity to SEQ ID NO: 122.

It will be understood by a person skilled in the art, that reference to amino acid residues within C62 to A81 of PilA is referring to the full-length PilA as defined in SEQ ID NO:56. Furthermore, reference to amino acid residues within C62 to A81 of PilA is referring to the amino acid number counting consecutively from the N-terminus of the amino acid sequence, of for example SEQ ID NO. 56. Amino acid residues within C62 to A81 refers to the amino acids from the 62^{nd} and 81^{st} amino acid of SEQ ID NO. 56.

A person skilled in the art will understand that when the PilA amino acid sequence is a variant and/or fragment of an amino acid sequence of SEQ ID NO. 56, such as an amino acid sequence at least 80%, 85%, 90%, 92%, 95%, 96%, 97%, 98% or 99% identical to SEQ ID NO. 56, the reference C62 to A81 refers to a the position that would be equivalent to the defined position, if this sequence was lined up with an amino acid sequence of SEQ ID NO. 56 in order to maximise the sequence identity between the two sequences.

Variants of SEQ ID NO.56 (e.g. SEQ ID NO: 57-119) could lead to a difference in the actual amino acid position of the consensus sequence in the sequence, however, by lining the sequence up with the reference sequence, the amino acid in in an equivalent position to the corresponding amino acid in the reference sequence can be identified and hence the appropriate amino acids identified.

In an embodiment the PilA antigen binding protein of the invention binds to PilA at one or more of amino acid residues within the region 62-81 (of SEQ ID NO: 56). In an embodiment the PilA antigen binding protein of the invention binds to PilA at one or more of amino acid residues within the region 62-81 of PilA (of SEQ ID NO: 56) or sequence with at least 70%, at least 80%, at least 90%, at least 95% or 100% identity to SEQ ID NO: 56. In an embodiment the PilA antigen binding protein of the invention binds to an epitope within or comprising amino acid residues 62-81 of PilA (of SEQ ID NO: 56). In an embodiment the PilA antigen binding protein of the invention binds to an epitope within or comprising amino acid residues 62-81 of PilA (of SEQ ID NO: 56) or sequence with at least 70%, at least 80%, at least 90%, at least 95% or 100% identity to SEQ ID NO: 56.

Reference to amino acid residues within C62 to A81 may also be referring to the corresponding residues within a fragment of PilA (such as the fragment described in SEQ ID NO: 120). This is only to the extent that the corresponding amino acids remain present in the sequence i.e. wherein said fragment retains the epitope of the invention. For example, within the fragment of PilA of SEQ ID NO: 120 (corresponding to amino acids 40-109 of SEQ ID NO: 56), the corresponding residues are C23 to A42.

Reference to amino acid residues C62 to A81 of PilA may also be referring to those corresponding residues within a fusion protein comprising PilA (wherein PilA may be fused to any heterologous polypeptide (for example Protein E)). This is only to the extent that the corresponding amino acids remain present in the fusion protein i.e. wherein said fusion retains the epitope of the invention. For example, within the PE-PilA fusion protein of SEQ ID NO: 122 (LVL-735 minus signal peptide), the corresponding residues are C166 to A185.

In an embodiment the PilA antigen binding protein of the invention binds to an epitope that is associated with an immunogenically active form of PilA. In an embodiment the PilA antigen binding protein of the invention binds to an epitope that is associated with PilA in a conformation where it is immunogenically active. In an embodiment the PilA antigen binding protein of the invention binds to an epitope that is associated with an immunogenically active form of a PE-PilA fusion protein (e.g. LVL-735 of SEQ ID NO: 122). In an embodiment the PilA antigen binding protein of the invention binds to an epitope within or comprising amino acid residues C62 to A81 of PilA wherein said epitope is associated with an immunogenically active form of PilA. In an embodiment the PilA antigen binding protein of the invention binds (or preferentially binds) to a PilA antigen which is capable of eliciting an immune response in a mammal, preferably in a human being. In an embodiment, the PilA antigen binding protein of the invention binds (or preferentially binds) to a PilA antigen which is protective against diseases associated with H. influenzae. In an embodiment, the PilA antigen binding protein of the invention binds (or preferentially binds) to a PilA antigen which is protective against AECOPD

In an embodiment the PilA antigen binding protein of the invention binds to an epitope that is associated with the native conformation of PilA. In an embodiment the PilA antigen binding protein of the invention binds to PilA in its native conformation with a higher specificity and/or higher affinity than to PilA in its non-native conformation. For example, the antigen binding protein binds to PilA in its native (or substantially native) conformation with higher specificity and/or higher affinity than to a PilA which is denatured. In an embodiment PilA may be denatured or adopt a non-native conformation for example via thermal stress, freeze-thawing, pH alterations, oxidation, enzymatic digestion (e.g. trypsin digestion), mishandling or process errors. In an embodiment PilA may be denatured or adopt a non-native conformation for example via reduction (e.g. methionine reduction) or via light exposure. In an embodiment PilA may be denatured via thermal stress. In an embodiment, PilA may be denatured following exposure to temperatures greater than room temperature (i.e. greater than approximately 20° C. to 22° C.), greater than 30° C., greater than 40° C., greater than 50° C., greater than 60° C. or greater than 70° C. In an embodiment, PilA may be denatured at 65° C.±5° C. In an embodiment, PilA may be denatured via thermal stress for up to 1 hour, up to 2 hours, up to 4 hours, up to 6 hours, up to 8 hours, up to 12 hours or up to 24 hours. In an embodiment, PilA may be denatured via thermal stress for greater than 24 hours.

In an embodiment the antigen binding protein binds to PilA in its native conformation with a higher specificity and/or affinity as compared to a vaccine sample comprising a PilA immunogen which has lost the relevant epitope (and thus function). In an embodiment, the PilA immunogen has lost the relevant epitope for example due to denaturation, aggregation or breakdown during storage or by mishandling.

In an embodiment the PilA antigen binding protein of the invention inhibits biofilm formation. Inhibition of biofilm formation can be measured for example using the methodology described in Example 2.

In an embodiment the PilA antigen binding protein of the invention is an antibody. In an embodiment the PilA antigen binding protein of the invention is a monoclonal antibody, optionally an IgG2A monoclonal antibody, optionally PEPilA/3 mAb. In an embodiment the PilA antigen binding protein of the invention is referred to as PEPILA/3 mAb.

In an embodiment the PilA antigen binding protein of the invention comprises: a variable heavy (VH) region comprising a sequence at least 80% identical to the sequence of SEQ ID NO: 161; and/or a variable light (VL) region comprising a sequence at least 80% identical to the sequence of SEQ ID NO: 163. In an embodiment the PilA antigen binding protein of the invention comprises: a variable heavy (VH) region comprising a sequence at least 80% identical to the sequence of SEQ ID NO: 161; and a variable light (VL) region comprising a sequence at least 80% identical to the sequence of SEQ ID NO: 163. For example, an antigen binding comprising: a variable heavy (VH) region comprising a sequence at least 80%, at least 85%, at least 90%, at least 91%, at least 92%, at least 93%, at least 94%, at least 95%, at least 96%, at least 97%, at least 98%, at least 99% or 100% identical to the sequence of SEQ ID NO: 161; and a variable light (VL) region comprising a sequence at least 80%, at least 85%, at least 90%, at least 91%, at least 92%, at least 93%, at least 94%, at least 95%, at least 96%, at least 97%, at least 98%, at least 99% or 100% identical to the sequence of SEQ ID NO: 163.

The PilA antigen binding protein sequence may be a variant sequence with up to 3 amino acid modifications. For example, the modification is a substitution, addition or deletion. For example, the variant sequence may have up to 3, 2 or 1 amino acid substitution(s), addition(s) and/or deletion(s). The sequence variation may exclude the CDR(s), for example the CDR(s) is intact, and the variation is in the remaining portion of the VH or VL (or HC or LC) sequence, so that the CDR sequence is fixed. The variant sequence substantially retains the biological characteristics of the unmodified antigen binding protein.

In an embodiment the PilA antigen binding protein of the invention comprises: a VH region comprising SEQ ID NO: 161; and/or a VL region comprising SEQ ID NO: 163. In an embodiment the PilA antigen binding protein of the invention comprises: a VH region comprising SEQ ID NO: 161; and a VL region comprising SEQ ID NO: 163. In an embodiment the PilA antigen binding protein of the invention comprises: a VH region consisting of SEQ ID NO: 161; and/or a VL region consisting of SEQ ID NO: 163. In an embodiment the PilA antigen binding protein of the invention comprises: a VH region consisting of SEQ ID NO: 161; and a VL region consisting of SEQ ID NO: 163.

In an embodiment the PilA antigen binding protein of the invention comprises a VH region encoded by SEQ ID NO: 160 and/or a VL region encoded by SEQ ID NO: 162.

There is also provided an antigen binding protein comprising any one or a combination of CDRs selected from CDR-H1, CDR-H2, CDR-H3 from SEQ ID NO: 161 , and/or CDR-L1, CDR-L2, CDR-L3 from SEQ ID NO: 163; or (ii) a CDR variant of (i), wherein the variant has 1, 2, or 3 amino acid modifications in each CDR, which is able to bind to PilA at one or more of amino acid residues within C62 to A81 of PilA (e.g. SEQ ID NO: 135). In an embodiment the PilA antigen binding protein of the invention comprises any one or a combination of CDRs selected from CDR-H1 (SEQ ID NO: 164), CDR-H2 (SEQ ID NO: 165) or CDR-H3 (SEQ ID NO: 166), and/or CDR-L1 (SEQ ID NO: 167), CDR-L2 (SEQ ID NO: 168) or CDR-L3 (SEQ ID NO: 169) or (ii) a CDR variant of (i), wherein the variant has 1, 2, or 3 amino acid modifications in each CDR, which is able to bind to PilA at one or more of amino acid residues within C62 to A81 of PilA (e.g. SEQ ID NO: 135).

The CDR regions may be identified using any method known to those of skill in the art. The CDR regions of the VH chain (of SEQ ID NO: 161) and VL chain (of SEQ ID NO: 163) are shown in Table 4 below.

TABLE 4
CDR Regions of the VH (SEQ ID NO: 161) and VL
(SEQ ID NO: 163) regions of the PilA antigen
binding protein of the invention.

| CDR | SEQ ID NO | Sequence |
|---|---|---|
| CDR-H1 | 164 | DYVIS |
| CDR-H2 | 165 | EIHPGSGSIHYNEKFKG |
| CDR-H3 | 166 | RGLRRPWFAY |
| CDR-L1 | 167 | RASKSVSTSGYSYMH |
| CDR-L2 | 168 | LASNLES |
| CDR-L3 | 169 | QHSRELPLT |

Immunogenic Compositions

The present invention further provides an immunogenic composition comprising the PE antigen binding protein of the invention and/or the PilA antigen binding protein of the invention. In an embodiment the immunogenic composition comprises the PE antigen binding protein of the invention and the PilA antigen binding protein of the invention. For example, the present invention provides an immunogenic composition comprising a PE antigen binding protein which binds to Protein E at one or more of amino acid residues within I41 to Y49 and Y141 to A154 of Protein E and a PilA antigen binding protein which binds to PilA at one or more of amino acid residues within C62 to A81 of PilA. For example, the present invention provides an immunogenic composition comprising a PE antigen binding protein which binds to Protein E at one or more of amino acid residues within I41 to Y49 and Y141 to A154 of Protein E. For example the present invention provides an immunogenic composition comprising a PilA antigen binding protein which binds to PilA at one or more of amino acid residues within C62 to A81 of PilA.

In a preferred embodiment, polysorbate 80 (for example, TWEEN (a US registered trademark) 80) is included within the immunogenic composition of the invention. In a further embodiment, a final concentration of about 0.03% to about 0.06% is used. Specifically, a final concentration of about 0.03%, 0.04%, 0.05% or 0.06% polysorbate 80 (w/v) may be used.

Formulations comprising the immunogenic compositions of the invention may be adapted for administration by an appropriate route, for example, by the intramuscular, sublingual, transcutaneous, intradermal or intranasal route. Such formulations may be prepared by any method known in the art.

In an embodiment the immunogenic composition of the invention may be administered with other antigens. For example, the present immunogenic composition may be administered with antigens from H. influenzae. For example, the present immunogenic composition may be administered with Protein D from H. influenzae.

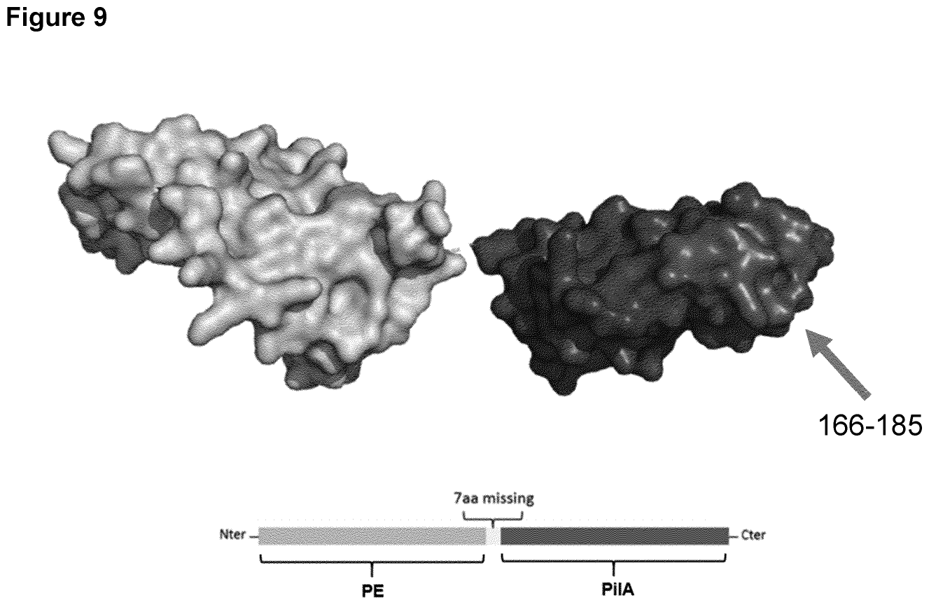

The immunogenic composition of the invention may further comprise protein D or an immunogenic fragment thereof from Haemophilus influenzae. Protein D (PD) is a highly conserved 42 kDa surface lipoprotein found in all Haemophilus influenzae, including nontypeable Haemophilus influenzae. Inclusion of this protein in the immunogenic composition may provide a level of protection against Haemophilus influenzae related otitis media [Wilkinson et al. Thorax. 2017; 72(10):919-27]. Suitable amino acid sequences for PD include, for example, the protein D sequence from FIG. 9 of EP0594610 (FIGS. 9a and 9b together, 364 amino acids) and as described in WO91/18926 or WO00/56360 (disclosed herein as SEQ ID NO: 138 and SEQ ID NO: 139).

Other suitable proteins may be encoded by, for example, GenBank accession numbers: X90493 (SEQ ID NO:140), X90489 (SEQ ID NO:141), X90491 (SEQ ID NO:142), Z35656 (SEQ ID NO:143), Z35657 (SEQ ID NO:144), Z35658 (SEQ ID NO:145), M37487 (SEQ ID NO:146). Protein D may be used as a full-length protein or as a fragment. For example, a protein D sequence may comprise (or consist) of the protein D fragment described in EP0594610 which begins at amino acid 20 of SEQ ID NO: 138 (i.e. the sequence SSHSSNMANT (SerSerHisSerSerAsnMetAlaAsnThr) (SEQ ID NO. 147), and lacks the 19 N-terminal amino acids from SEQ ID NO: 138, optionally with the tripeptide MDP from NS1 fused to the N-terminal of said protein D fragment (348 amino acids) (SEQ ID NO: 139). In one aspect, the protein D or fragment of protein D is unlipidated.

One skilled in the art will further recognise that immunogenic compositions may comprise polypeptides having sequence identity to Protein D provided that such polypeptides are capable of generating an immune response to Protein D, for example, they comprise one or more epitopes of protein D. Thus, immunogenic compositions may comprise an isolated immunogenic polypeptide having sequence identity of at least 70%, 80%, 85%, 90%, 91%, 92%, 93%, 94%, 95%, 96%, 97%, 98%, 99%, or 100% to SEQ ID NO:139. In an embodiment, the isolated immunogenic polypeptide is capable of eliciting an immune response against SEQ ID NO:139, particularly an immune response that results in the formation of antibodies that bind to SEQ ID NO:139. In an embodiment the immunogenic composition of the invention comprises SEQ ID NO. 139 which corresponds to the protein D sequence from FIG. 9 of EP0594610 (FIGS. 9a and 9b together, 364 amino acids).

The amount of the immunogenic composition which is required to achieve the desired therapeutic or biological effect will depend on a number of factors such as the use for which it is intended, the means of administration, the recipient and the type and severity of the condition being treated and will be ultimately at the discretion of the attendant physician or veterinarian. In general, a typical dose for the treatment of a condition caused in whole or in part by H. influenzae in a human, for instance, may be expected to lie in the range of from about 0.001 mg-0.120 mg. More specifically, a typical dose for the treatment of a condition caused wholly or in part by H. influenzae in a human may lie in the range of from about 0.003 mg to about 0.03 mg of protein. The present invention provides an immunogenic composition comprising the antigen binding protein of the invention for use in the treatment or prevention of a condition or disease caused wholly or in part by H. influenzae. The immunogenic composition may contain additional antigens; a typical dose for the treatment of a condition caused wholly or in part by H. influenzae in a human may lie in the range of from about 0.005 mg to about 0.05 mg for each additional antigen. This dose may be administered as a single unit dose. Several separate unit doses may also be administered. For example, separate unit doses may be administered as separate priming doses within the first year of life or as separate booster doses given at regular intervals (for example, every 1, 5 or 10 years). The present invention also provides an immunogenic composition comprising the antigen binding protein of the invention or a for use in the treatment or prevention of a condition or disease caused wholly or in part by Haemophilus influenzae in combination with at least one antigen from Moraxella catarrhalis.

In an embodiment, the immunogenic composition of the invention may further comprise an antigen from Moraxella catarrhalis. In an embodiment said antigen from Moraxella catarrhalis is UspA2. In an embodiment said antigen is a fragment of UspA2.

As used herein “UspA2” means Ubiquitous surface protein A2 from Moraxella catarrhalis. UspA2 may consist of or comprise the amino acid sequence of SEQ ID NO: 148 (UspA2 from ATCC 25238) as well as sequences with at least or exactly 63%, 66%, 70%, 72%, 74%, 75%, 77%, 80%, 84%, 85%, 90%, 95%, 96%, 97%, 98%, 99% or 100% identity, over the entire length, to SEQ ID NO: 148.

Immunogenic fragments of UspA2 may comprise immunogenic fragments of at least 450, 490, 511, 534 or 535 contiguous amino acids of SEQ ID NO: 148. Immunogenic fragments of UspA2 may comprise immunogenic fragments of UspA2, for example any of the UspA2 constructs shown in Table 2 below (and as disclosed in WO2015/125118 A1). Immunogenic fragments of UspA2 may comprise a methionine at the amino terminal and/or 0, 1, 2, 3, 4, 5, 6 histidine tag residues. The immunogenic fragments may elicit antibodies which can bind the full-length sequence from which the fragment is derived.

TABLE 2
Immunogenic Fragments/Constructs of
UspA2 from Moraxella catarrhalis.

| | | Construct (UspA2 numbering |
|---|---|---|
| Fragment | SEQ ID NO: | according to SEQ ID NO: 1) |
| MC-001 | SEQ ID NO: 149 | (M)(UspA2 amino acids 30-540) |
| | | (ASHHHHHH) |
| MC-002 | SEQ ID NO: 150 | (M)(UspA2 amino acids 30-540) |
| MC-003 | SEQ ID NO: 151 | (M)(UspA2 amino acids 30-540)(H) |
| MC-004 | SEQ ID NO: 152 | (M)(UspA2 amino acids 30-540)(HH) |
| MC-005 | SEQ ID NO: 153 | (M)(UspA2 amino acids 30-519) |
| | | (ASHHHHHH) |
| MC-006 | SEQ ID NO: 154 | (M)(UspA2 amino acids 30-519) |
| MC-007 | SEQ ID NO: 155 | (M)(UspA2 amino acids 30-564) |
| | | (ASHHHHHH) |
| MC-008 | SEQ ID NO: 156 | (M)(UspA2 30-564)(HH) |
| MC-009 | SEQ ID NO: 157 | (M)(UspA2 31-564)(HH) |
| MC-010 | SEQ ID NO: 158 | (M)(UspA2 amino acids 30-564) |
| MC-011 | SEQ ID NO: 159 | (M)(UspA2 amino acids 31-540) |
| | | (ASHHHHHH) |

(M) = Methionine,
(ASHHHHHH) = Ala, Ser, His, His, His, His, His, His,
(H) = His,
(HH) = His His

In another aspect of the invention, the immunogenic composition comprises an immunogenic fragment of UspA2, suitably an isolated immunogenic polypeptide with at least 70%, 80%, 85%, 90%, 91%, 92%, 93%, 94%, 95%, 96%, 97%, 98%, 99%, or 100% identity to a polypeptide selected from the group consisting of SEQ ID NO: 149 to SEQ ID NO: 159. In an embodiment the immunogenic composition of the invention comprises an isolated immunogenic polypeptide with at least 70%, 80%, 85%, 90%, 91%, 92%, 93%, 94%, 95%, 96%, 97%, 98%, 99%, or 100% identity to MC-009 (SEQ ID NO. 157) which corresponds to SEQ ID NO: 69 of WO2015/125118 A1. In an embodiment the immunogenic composition of the invention comprises MC-009 (SEQ ID NO. 157) which corresponds to SEQ ID NO: 69 of WO2015/125118 A1.

Vaccines

The invention further provides a vaccine comprising the PE antigen binding protein of the invention and/or the PilA antigen binding protein of the invention. The invention further provides a vaccine comprising the PE antigen binding protein of the invention and the PilA antigen binding protein of the invention For example, in an embodiment the present invention provides a vaccine comprising a PE antigen binding protein which binds to Protein E at one or more of amino acid residues within I41 to Y49 and Y141 to A154 of Protein E and a PilA antigen binding protein which binds to PilA at one or more of amino acid residues within C62 to A81 of PilA.

In an embodiment the vaccine of the invention further comprises an adjuvant (e.g. AS01E). Immunogenic compositions and vaccines of the invention will generally comprise one or more adjuvants.

Suitable adjuvants include an aluminium salt such as aluminium hydroxide gel or aluminium phosphate or alum, but may also be a salt of calcium, magnesium, iron or zinc, or may be an insoluble suspension of acylated tyrosine, or acylated sugars, cationically or anionically derivatized saccharides, or polyphosphazenes. In one embodiment, the protein may be adsorbed onto aluminium phosphate. In another embodiment, the protein may be adsorbed onto aluminium hydroxide. In a third embodiment, alum may be used as an adjuvant.

Suitable adjuvant systems which promote a predominantly Th1 response include: non-toxic derivatives of lipid A, Monophosphoryl lipid A (MPL) or a derivative thereof, particularly 3-de-O-acylated monophosphoryl lipid A (3D-MPL) (for its preparation see GB2220211A); and a combination of monophosphoryl lipid A, preferably 3-de-O-acylated monophosphoryl lipid A, together with either an aluminium salt (for instance aluminium phosphate or aluminium hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen [Thoelen et al. Vaccine. 1998; 16(7):708-14 (also see EP689454B1)].

AS01 is an Adjuvant System containing MPL (3-O-desacyl-4′-monophosphoryl lipid A), QS21 ((Quillaja saponaria Molina, fraction 21) Antigenics, New York, NY, USA) and liposomes. AS01B is an Adjuvant System containing MPL, QS21 and liposomes (50 μg MPL and 50 μg QS21). AS01E is an Adjuvant System containing MPL, QS21 and liposomes (25 μg MPL and 25 μg QS21). In one embodiment, the immunogenic composition or vaccine of the invention comprises AS01. In another embodiment, the immunogenic composition or vaccine of the invention comprises AS01B or AS01E. In an embodiment, the immunogenic composition or vaccine comprises AS01E.

AS02 is an Adjuvant System containing MPL and QS21 in an oil/water emulsion. AS02V is an Adjuvant System containing MPL and QS21 in an oil/water emulsion (50 μg MPL and 50 μg QS21).

AS03 is an Adjuvant System containing α-Tocopherol and squalene in an oil/water (o/w) emulsion. AS03A is an Adjuvant System containing α-Tocopherol and squalene in an o/w emulsion (11.86 mg tocopherol). AS03B is an Adjuvant System containing α-Tocopherol and squalene in an o/w emulsion (5.93 mg tocopherol). AS03C is an Adjuvant System containing α-Tocopherol and squalene in an o/w emulsion (2.97 mg tocopherol). In one embodiment, the immunogenic composition or vaccine comprises AS03.

AS04 is an Adjuvant System containing MPL (50 μg MPL) adsorbed on an aluminium salt (500 μg Al3+). In one embodiment, the immunogenic composition or vaccine comprises AS04.

A system involving the use of QS21 and 3D-MPL is disclosed in WO 94/00153. A composition wherein the QS21 is quenched with cholesterol is disclosed in (49). An additional adjuvant formulation involving QS21, 3D-MPL and tocopherol in an oil in water emulsion is described in WO 95/17210. In one embodiment the immunogenic composition additionally comprises a saponin, which may be QS21. The formulation may also comprise an oil in water emulsion and tocopherol WO 95/17210. Unmethylated CpG containing oligonucleotides (WO 96/02555) and other immunomodulatory oligonucleotides (WO 0226757 and WO 03507822) are also preferential inducers of a TH1 response and are suitable for use in the present invention.

Additional adjuvants are those selected from the group of metal salts, oil in water emulsions, Toll like receptor agonists, (in particular Toll like receptor 2 agonist, Toll like receptor 3 agonist, Toll like receptor 4 agonist, Toll like receptor 7 agonist, Toll like receptor 8 agonist and Toll like receptor 9 agonist), saponins or combinations thereof.

In an embodiment the vaccine comprises at least one additional antigen from H. influenzae e.g. protein D or an immunogenic fragment thereof. In an embodiment the vaccine comprises excipients. Possible excipients include arginine, pluronic acid and/or polysorbate.

Uses and Methods of Treatment

Another aspect of the invention provides an antigen binding protein, immunogenic composition or vaccine of the invention for use in therapy.

Another aspect of the invention provides an antigen binding protein, immunogenic composition or vaccine of the invention for use in treating or preventing an infection, disease or condition caused by H. influenzae.

Another aspect of the invention provides an antigen binding protein, immunogenic composition or vaccine of the invention for use in treating or preventing an infection, disease or condition which is otitis media, pneumonia and/or acute exacerbations of chronic obstructive pulmonary disease (AECOPD).

Another aspect of the invention provides an antigen binding protein, immunogenic composition or vaccine of the invention for use in treating or preventing an infection, disease or condition caused by H. influenzae, in a mammal, particularly a human.

Another aspect of the invention provides an antigen binding protein, immunogenic composition or vaccine of the invention, for use in the treatment or prevention of acute otitis media, pneumonia and/or acute exacerbations of chronic obstructive pulmonary disease (AECOPD).

Another aspect of the invention provides an antigen binding protein, immunogenic composition or vaccine of the invention for use in the manufacture of a medicament for the treatment or prevention of an infection, disease or condition caused by H. influenzae.

Another aspect of the invention provides an antigen binding protein, immunogenic composition or vaccine of the invention for use in the manufacture of a medicament for the treatment or prevention of pneumonia, otitis media and/or acute exacerbations of chronic obstructive pulmonary disease AECOPD.

Another aspect of the invention provides a method of treatment or prevention of an infection, disease or condition caused by H. influenzae, in a subject in need thereof comprising administering to said subject a therapeutically effective amount of an antigen binding protein, immunogenic composition or vaccine of the invention.

Another aspect of the invention provides a method of treatment of a disease or condition caused by/attributable to, resulting from a H. influenzae infection, in a subject at risk for or having a H. influenzae infection, comprising administering to said subject a therapeutically effective amount of an antigen binding protein, immunogenic composition or vaccine of the invention.

Another aspect of the invention provides a method of treatment or prevention of acute exacerbations of chronic obstructive pulmonary disease (AECOPD), pneumonia and/or otitis media in a subject in need thereof comprising administering to said subject a therapeutically effective amount of an antigen binding protein, immunogenic composition or vaccine of the invention.

Otitis Media

Otitis media is a major cause of morbidity in 80% of all children less than 3 years of age [Expert Rev. Vaccines 5:517-534 (2006)]. More than 90% of children develop otitis media before age 7 [Current Opinion in Investigational Drugs 4:953-958 (2003)]. In 2000, there were 16 million visits made to office-based physicians for otitis media in the United States and approximately 13 million antibacterial prescriptions dispensed [Pediatrics 113:1451-1465 (2004)]. In European countries, the reported acute otitis media rates range between 0.125 to 1.24 per child-year [Expert Review of Vaccines 8:1479-1500 (2009)]. Otitis media is a costly infection and the most common reason children receive antibiotics [Current Infectious Disease Reports 11:177-182 (2009)]. Bacteria are responsible for approximately 70% of cases of acute otitis media, with Streptococcus pneumoniae, non-typeable Haemophilus influenzae, and Moraxella catarrhalis predominating as the causative agents [Expert Review of Vaccines 5:517-534 (2006)]. A subset of children experience recurrent and chronic otitis media and these otitis prone children have protracted middle-ear effusions that are associated with hearing loss and delays in speech and language development [Current Infectious Disease Reports 11:177-182 (2009)].

Following the introduction of the heptavalent pneumococcal vaccine in many countries, some studies have demonstrated a significant increase in the proportion of acute otitis media caused by H. influenzae, with H. influenzae becoming the predominant pathogen [Pediatric Infectious Disease Journal 23:824-828; Pediatric Infectious Disease Journal 23:829-833 (2004)].

Since otitis media is a multifactorial disease, the feasibility of preventing otitis media using a vaccination strategy has been questioned [Current Infectious Disease Reports 11:177-182 (2009)]. However, the results from one study suggest that it is possible for an antigen to induce at least partial protection against non-typeable H. influenzae [Lancet 367:740-748 (2006)]. One approach to developing vaccine antigens is to use antigenically conserved regions of genetically heterogeneous but abundantly expressed surface molecules. Another approach is to identify surface proteins that demonstrate sequence or functional epitope conservation. A third consideration for a vaccine antigen could be to select an antigen that is expressed during infection and colonization in a human host. Murphy and colleague state that, despite the existence of several potential non-typeable H. influenzae candidate antigens, one cannot predict with certainty whether the candidate antigen will be effective [Current Infectious Disease Reports 11:177-182 (2009)]. Some of the proteins described as potential vaccine antigens are: Haemophilus adhesin protein (Hap), High molecular-weight (HMW) proteins 1 and 2, H. influnzae adhesin (Hia), D15 protein, HtrA heat shock protein, P2 surface protein, lipoprotein D, P5 fimbrin derived peptides, outer membrane protein P4, outer membrane protein (OMP) 26 (OMP26), P6 protein, Protein E, Type IV pilus, lipooligosaccharide and phosphoryl choline [Current Infectious Disease Reports 11:177-182 (2009); Expert Review of Vaccines 5:517-534 (2006)].

The chinchilla model is a robust and validated animal model of otitis media and its prevention [Expert Review of Vaccines 8:1063-1082 (2009)]. While the chinchilla model may mimic the natural course of human infection, others have suggested that results in the chinchilla model may vary from one laboratory to the next [Current Opinion in Investigational Drugs 4:953-958 (2003)].

Various other rodents have also been used for the induction of otitis media and are summarized in [Vaccine 26:1501-1524 (2008)]. The murine animal model is often studied in otitis media research.

The presence of bactericidal antibody is associated with protection from otitis media due to non-typeable H. influenzae [Current Opinion in Infectious Disease 16:129-134 (2003)]. However, an immune response need not be bactericidal to be effective against NTHi. Antibodies that merely react with NTHi surface adhesins can reduce or eliminate otitis media in the chinchilla [Current Opinion in Investigational Drugs 4:953-958 (2003)].

Thus, in an embodiment the present invention provides a method of treatment or prevention of otitis media in a subject in need thereof comprising administering to said subject a therapeutically effective amount of an antigen binding protein, immunogenic composition or vaccine of the invention.

AECOPD

Chronic Obstructive Pulmonary Disease (COPD), a common preventable disease, is characterised by persistent airflow limitation that is usually progressive. The airflow limitation is associated with an enhanced chronic inflammatory response in the airways and lungs to noxious particles of gases. The most important environmental risk factor for COPD is tobacco smoking, even though other factors, such as occupational exposure, may also contribute to the development of the disease. It is a multi-component disease that manifests as an accelerated decline in lung function, with symptoms such as breathlessness on physical exertion, deteriorating health status and exacerbations.

The prevalence of COPD is increasing: worldwide, COPD (GOLD grade II and above) affects 10.1±4.8% of the population ≥40 years of age [Buist A S et al. Lancet. 2007; 370(9589):741-50]. COPD is most prevalent in adults/elderly with a history of smoking [Mannino D M et al. Respir Care. 2002; 47(10):1184-99]. COPD affects 24 million Americans and is the third leading cause of death in the US and the world [Decramer et al. 2012; Burney Eur Respir J 2015; GBD 2015 Chronic Respiratory Disease Collaborators. Lancet Respir Med 2017; López-Campos J L et al. Respirology 2016]. Recent papers report that in 2015, COPD ranked third among the global age-standardised death rates for both sexes, with about 3.2 million patients dying of the disease [Lancet. 2016; 388(10053):1459-544].

Acute exacerbations and comorbidities contribute to the overall disease severity in individual COPD patients. An acute exacerbation of COPD (AECOPD) is an acute event characterised by a worsening of the patient's respiratory symptoms that is beyond normal day-to-day variations and leads to a change in medication. AECOPD increases morbidity and mortality, leading to faster decline in lung function, poorer functional status [Sapey E, Stockley R A. aetiology. Thorax. 2006; 61(3):250-8].

The lungs are known to be colonised with different strains of bacteria [Erb-Downward J R et al. PLoS One. 2011; 6(2) and Wilkinson T M et al. Chest. 2006; 129(2):317-24]. In COPD patients, acquisition of new bacterial strains is believed to be an important cause of AECOPD [Sethi et al N Engl J Med. 2002; 347(7):465-71]. Although estimates vary widely, Non-Typeable Haemophilus influenzae (NTHi) appears to be the main bacterial pathogen associated with AECOPD (11-38%), followed by Moraxella catarrhalis (3-25%) and Streptococcus pneumoniae (4-9%) [Alamoudi O S et al. Respirology. 2007; 12(2):283-7, Bandi Vet al. FEMS Immunol Med Microbiol. 2003; 37(1):69-75 and Beasley V et al. Int J Chron Obstruct Pulmon Dis. 2012; 7:555-69].

Thus, in an embodiment the present invention provides a method of treatment or prevention of AECOPD in a subject in need thereof comprising administering to said subject a therapeutically effective amount of an antigen binding protein, immunogenic composition or vaccine of the invention.

Pneumonia

Community-acquired pneumonia (CAP) has been described as the leading cause of death from infectious disease and the six-ranked cause of death overall in the United States. Moraxella catarrhalis is one of the pathogens associated with CAP in North America [Alcon A, Fabregas N, Torres A. Pathophysiology of pneumonia. Clin Chest Med. 2005; 26(1):39-46] and is one of the pathogens associated with moderate to severe community acquired pneumonia in Japan [Takaki M et al. Jpn J Infect Dis. 2014; 67(4):269-75]. Moraxella catarrhalis may be especially likely to cause pneumonia, endocarditis, septicaemia and meningitis in immunocompromised subjects. As well as being a primary causative, in some instance pneumonia in both adults and children can be exacerbated by Moraxella catarrhalis infection and pneumonia in children can be complicated by bacteraemia (presence of bacteria in blood) following bacterial infection of Moraxella catarrhalis. In addition to M. catarrhalis, non-typeable H. influenzae (NTHI) is regarded the most common cause of invasive haemophilus infection in all ages. NTHI are a recognized cause of bacteraemic and non-bacteraemic pneumonia in children and in adults [Mary P E Slack, Pneumonia (2017) 9:9].

Thus, in an embodiment the present invention provides a method of treatment or prevention of pneumonia in a subject in need thereof comprising administering to said subject a therapeutically effective amount of an antigen binding protein, immunogenic composition or vaccine of the invention.

Assay

The role of a potency assay is to ensure that an antigen contains the appropriate biochemical properties to elicit the needed immune response. The in vitro relative potency assay described herein may be used for drug-product release and stability testing of an NTHi-Mcat vaccine.

In a further aspect of the invention there is provided the use of the PE antigen binding protein of the invention in the detection of, or measurement of a change in, the conformation of Protein E. In an embodiment said PE antigen binding protein is used in the detection of, or measurement of a change in the conformation of Protein E by measuring the binding of the PE antigen binding protein of the invention to an epitope within the region I41 to Y49 and Y141 to A154 of Protein E. In a further aspect of the invention there is provided the use of the PilA antigen binding protein of the invention in the detection of, or measurement of a change in, the conformation of PilA. In a further aspect there is provided the use of both the PE antigen binding protein of the invention and the PilA antigen binding protein of the invention in the detection of, or measurement of a change in, the conformation of a test antigen, optionally a PE-PilA fusion protein, optionally the PE-PilA fusion protein of SEQ ID NO: 122 (LVL-735). In an embodiment there is provided the use of both the PE antigen binding protein of the invention and the PilA antigen binding protein of the invention in the detection of, or measurement of a change in, the conformation of a PE-PilA fusion protein with at least 80% (e.g. at least 85%, at least 90%, at least 95% or at least 100%) identity to SEQ ID NO: 122.

Because detection of, or measurement of a change in the conformation of Protein E and PilA relies upon the PE and PilA antigen binding proteins of the invention, said use is considered to be a good predictor of in vivo potency as said antigen binding proteins comprise biologically relevant, functional epitopes. In an embodiment, determining or measuring the presence of Protein E and/or PilA in its native conformation involves determining or measuring the presence of Protein E and/or PilA in a form which is suitable for administration to a patient (e.g. as a component of an immunogenic composition).

In a further aspect, there is provided an assay to determine or measure potency with respect to Protein E using the PE antigen binding protein of the invention. In a further aspect, there is provided an assay to determine or measure potency with respect to PilA using the PilA antigen binding protein of the invention. In a further aspect there is provided an assay to determine potency with respect to PE-PilA using the PE antigen binding protein of the invention and the PilA antigen binding protein of the invention.

In such cases where neutralizing or functional antibodies (e.g. mAb) have been identified, an in vitro relative potency assay can be developed in which capture and detection antibodies against two different epitopes are used in a sandwich ELISA. For a Sandwich ELISA IVRP assay to function, the target antigen must contain at least two antigenic sites capable of binding to antibodies. In an embodiment the target antigen is a PE-PilA fusion protein wherein one antigenic site is on Protein E and one antigenic site is on PilA. Monoclonal or polyclonal antibodies can be used as the capture and detection antibodies in sandwich ELISA although monoclonal antibodies may allow quantification of smaller differences. If the antigen is not present in the sample (or if the antigen is suboptimal or denatured and has thus not bound to the capture antibody), then the detection antibody will not have anything to bind to and no signal will be produced. Dilution curves of vaccine test samples are compared against a reference curve by parallel-line analysis. The relative potency could then be determined by multiplying the reference standard potency by the ratio of the sample ED₅₀ versus the reference ED₅₀. Generally, a low sample ED₅₀ indicates lower vaccine potency as more vaccine antigens were needed to achieve the same assay signal.

The potency of the test antigen (i.e. PE-PilA fusion protein, for example SEQ ID NO: 122) is therefore determined relative to a reference sample, wherein the reference sample is a sample which has been tested in vivo (specifically in humans) and which has shown clinicial efficacy. For example the reference sample may be a clinical lot of PE-PilA fusion protein (for example SEQ ID NO: 122) that was tested and demonstrated to be efficacious in the proof-of-concept clinical study to determine vaccine efficacy. The test antigen may be a PE-PilA fusion protein (e.g. SEQ ID NO: 122) which has been newly manufacturerd and is thus ready for release to the public subject to passing the IVRP assay of the invention.

A further aspect of the invention therefore provides an assay comprising exposing a sample of a test antigen to an antigen binding protein of the invention and measuring the amount of antigen binding protein bound to the test antigen. In an embodiment the antigen binding protein of the invention is the PE antigen binding protein of the invention. In an embodiment the antigen binding protein of the invention is the PilA antigen binding protein of the invention. In an embodiment there is provided an assay comprising exposing a sample of a test antigen to both the PE antigen binding protein of the invention and the PilA antigen binding protein of the invention and measuring the amount of antigen binding protein bound to the test antigen.

In an embodiment the test sample comprises Protein E and/or PilA. In an embodiment the test sample is a sample containing Protein E, a fragment of Protein E or a fusion protein comprising Protein E and/or a sample containing PilA, a fragment of PilA or a fusion protein comprising PilA. In an embodiment said test antigen (i.e. said sample containing Protein E and/or PilA) is an immunologically active sample. In an embodiment said test antigen (e.g. said sample containing Protein E and/or PilA) is capable of eliciting an immune response when administered to a human.

In an embodiment the assay comprises exposing a sample of a test antigen to a PE or PilA antigen binding protein of the invention and measuring the specificity of antigen binding protein bound to the test antigen.

In an embodiment the assay of the invention is an in vitro assay. In an embodiment the assay of the invention is an enzyme linked immunosorbent assay (ELISA), optionally a sandwich ELISA. In an embodiment the assay of the invention is a sandwich ELISA. The sandwich ELISA assay uses antigen specific antibodies to measure the concentration of immune-dominaint functional epitopes in the vaccine sample. The assay of the invention is intended for use in measuring the potency of vaccine material for human use (e.g. of clinical trial material as well as commercial use).

In an embodiment the assay of the invention is a sandwich ELISA using the PE antigen binding protein of the invention and the PilA antigen binding protein of the invention. In an embodiment the assay of the invention is a sandwich ELISA using an antigen binding protein which binds to Protein E at one or more of the amino acid residues within I41 to Y49 and Y141 to A154 and an antigen binding protein which binds to PilA at one or more of the amino acid residues within C62 to A81 of PilA. In an embodiment the assay of the invention is a sandwich ELISA using the ProtE/5 mAb and the PE-PilA/3 mAb.

In an embodiment the binding of the test antigen to the antigen binding protein of the invention may be achieved following pre-coating of a microtiter plate with a capture antibody.

In one aspect, the assay of the invention is an in vitro Relative Potency (IVRP) assay of Protein E and/or PilA. In an embodiment the assay of the invention is a sandwich ELISA based IVRP which is used to test the potency of the PE-PilA fusion protein of SEQ ID NO: 122. In an embodiment, the assay of the invention is a sandwich ELISA using two antibodies, one antibody specific for a functional epitope on PE and one antibody specific for a functional epitope on PilA.

In an embodiment the PE antigen binding protein of the invention (e.g. ProtE/5 mAb) is the capture antibody. In an embodiment, the PE antigen binding protein of the invention (e.g. ProtE/5 mAb) is the detection antibody. In an embodiment the PilA antigen binding protein of the invention is the capture antibody. In an embodiment the PilA antigen binding protein of the invention is the detection antibody.

In an embodiment the PE antigen binding protein of the invention is the capture antibody and the PilA antigen binding protein of the invention is the detection antibody. Reference to capture/detection antibodies may also relate to capture/detection antigen binding proteins.

In an embodiment, the detection antibody is biotinylated. As used herein the term “biotinylated” refers to a protein, nucleic acid or other molecule (e.g. antibody or secondary antibody) which has undergone a process wherein biotin is covalently attached to it. Biotin can be bound by avidins and streptavidin with high affinity. Streptavidin can be conjugated to a detection system (e.g. peroxidase-conjugated streptavidin) enabling quantification of bound antibody. For example, peroxidase-conjugated streptavidin binds to a biotinylated secondary antibody and the conjugated peroxidase (e.g. horseradish peroxidase) provides enzyme activity for detection using an appropriate substrate system.

In an embodiment the detection antibody is labeled with an enzyme wherein the amount of test antigen bound to the detection antibody is determined by measuring the conversion of a substrate into a detectable product by said enzyme. In an embodiment the detection antibody is labeled with an enzyme that is conjugated to streptavidin. In an embodiment the enzyme that is conjugated to streptavidin is peroxidase. In an embodiment a substrate is utilized which, upon interaction with peroxidase, causes a detectable change, optionally a change in absorbance or fluorescence. In an embodiment the substrate is o-phenylenediamine dihydrochloride (OPD).

In other words, the amount of test antigen bound to the antigen binding protein is determined by measuring the enzymatic conversion of a o-phenylenediamine dihydrochloride (OPD) as peroxidase substrate into a detectable product. In an embodiment the ODP peroxidase substrate is oxidized to the product 2,3-diaminophenazine. In an embodiment the oxidized product is detectable spectrophotometrically at 450 nm or 492 nm. In an embodiment, the oxidized product is detectable by measuring absorbance at 490 nm and 620 nm. In an embodiment the peroxidase (enzyme) and ODP (substrate) reaction can be stopped with HCl or H₂SO₄ for example the reaction can be stopped with 3M of HCl or H₂SO₄ or for example with 1N chloridic acid.

Other suitable detection systems include as follows; P-Nitrophenyl-phosphate (pNPP) for an alkaline phosphatase enzyme detection system, hydrogen peroxide for a horseradish peroxidase detection system, TMB (3,3′,5,5′-tetramethylbenzidine), and ABTS (2,2′-azino-di-[3-ethyl-benzothiazoline-6 sulfonic acid] diammonium salt). It is foreseen that any suitable detection system could be utilised, in order to quantify the ELISA. For example, any chromogenic, chemiluminescent, or fluorescent readout from the enzyme-substrate interaction or excited fluorophore could be utilised.

In an alternative embodiment the detection antibody is labeled with a detectable substance and wherein the amount of antigen binding protein bound to the test antigen is determined by measuring the amount of detectable substance associated with the test antigen sample upon exposure to the antigen binding protein.

In detail, the invention provides a binding immunoassay. The invention can use any ELISA format, including those conventionally known as direct ELISA, indirect ELISA, sandwich ELISA, and competitive ELISA. Step (i) of the ELISA assay of the invention involves permitting a Protein E, PilA or PE-PilA antigen within a sample to interact with an antibody (optionally a monoclonal antibody). The interaction between the antibody and the immunogen is then detected in step (ii). The interaction can be measured quantitatively, such that step (ii) provides a result which indicates the concentration of the antibody's target epitope within the vaccine sample.

By using a monoclonal antibody which binds to a bactericidal or conformational epitope, the result in step (ii) indicates the concentration of the corresponding functional epitope in the vaccine sample and can distinguish between immunogens which retain the relevant epitope (and function) and those which have lost the epitope (e.g. due to denaturation, aggregation or breakdown during storage or by mishandling). By comparison with values obtained with a standard vaccine of known potency, results from step (ii) can be used to calculate relative potency of a test vaccine.

Labelling of antibodies in an ELISA can take various forms. In an ELISA the antibody is labeled with an enzyme, which is then used to catalyze a reaction whose product is readily detectable. The linked enzyme can cause a detectable change in an enzyme substrate which is added to the labeled antibody after it becomes immobilized e.g. to modify a substrate in a manner which causes a colour change. For example, the enzyme may be a peroxidase (e.g. horseradish peroxidase, HRP), or a phosphatase (e.g. alkaline phosphatase, AP). Other enzymes can also be used e.g. laccase, β-galactosidase, etc.

The choice of substrate will depend on the choice of linked enzyme. Preferred substrates undergo a colorimetric change, a chemiluminescent change, or a chemifluorescent change when contacted with the linked enzyme. Colorimetric substrates (and their enzymatic partners) include but are not limited to: PNPP or p-Nitrophenyl Phosphate (AP); ABTS or 2,2′-Azinobis [3-ethylbenzothiazoline-6-sulfonic acid] (HRP); OPD or o-phenylenediamine dihydrochloride (HRP); and TMB or 3,3′,5,5′-tetramethylbenzidine (HRP). Chemiluminescent substrates include luminol or 5-amino-2,3-dihydro-I,4-phthalazinedione (HRP), particularly in the presence of modified phenols such as p-iodophenol. Chemifluorescent substrates include p-hydroxyhydrocinnamic acid. Various proprietary substrates are also available, and these can be used with the invention if desired e.g. QuantaBlu, QuantaRed, SuperSignal, Turbo TMB, etc.

Where an ELISA reagent is immobilized on a solid surface, this surface take various forms. Usually the reagent is immobilized on a plastic surface, such as a surface made from polystyrene, polypropylene, polycarbonate, or cyclo-olefin. The plastic will usually be transparent and colourless, particularly when using chromogenic enzyme substrates. White or black plastics may be preferred used when using luminescent or fluorescent substrates, as known in the art. The plastic will generally be used in the form of a microwell plate (microtiter plate) as known in the art for ELISA (a flat plate having multiple individual and reaction wells). Such plates include those with 6, 24, 96, 384 or 1536 sample wells, usually arranged in a 2:3 rectangular matrix. Microwell plates facilitate the preparation of dilution series and also the transfer of materials from one plate to another while maintaining spatial relationships e.g. in the step of transferring a mixture of antibody and vaccine into a different microwell plate for measuring the interaction between the antibody and vaccine.

During an ELISA it may be desirable to add a blocking reagent and/or detergent e.g. to reduce non-specific binding interactions which might distort the assay's results. Blocking procedures are familiar to people working in the ELISA field. In an embodiment the assay of the invention uses a 1% bovine serum albumin blocking solution to reduce non-specific binding.

In addition to the ELISA formats discussed above, the invention can also be extended to use alternatives to ELISA, such as flow injection immunoaffinity analysis (FIIAA), AlphaLISA or AlphaScreen [6], dissociation-enhanced lanthanide fluorescent immunoassay (DELFIA), ELAST, the BIO-PLEX Suspension Array System, MSD, etc. Any suitable antibody-antigen complex binding assays can be used.

In an embodiment the assay of the invention may be carried out using the GYROLAB system. The GYROLAB system is a fully automated nanoliter-scale immunoassay platform containing streptavidin-coated microfluidic columns in a compact-disc (CD) technology format. The GYROLAB Bioaffy CD contains 96 to 112 streptavidin-coated columns inside microstructures. Sequential addition of reagents and samples in each microstructure is fully automated. Added capture reagent is first stopped by hydrophobic breaks and centrifugal force due to the rotation of the CD drives reagent into colums and it binds to streptavidin-coated particles. Samples and detection reagent are then applied to activated columns and immuno-sandwiches are assembled. The GYROLAB system, including preparation of its microfluidic affinity columns is described on the www.gyros.com website.

The IVRP assay of the invention may be carried out using the GYROLAB system (i.e PE-PilA IVRP GYROLAB assay). The PE-PilA IVRP GYROLAB assay uses a biotinylated mouse anti-PE monoclonal antibody (mAb ProtE/5) as the capture antibody and an Alexa Fluor647-labeled mouse anti-PilA monoclonal antibody (mAb PEPilA/3) as the detection antibody.

The PE-PilA IVRP GYROLAB assay substantially comprises the following steps:
i) All reagents, reference standard and samples are diluted to defined working concentrations.
ii) The GYROLAB Bioaffy 1000 CD contains 96 streptavidin-coated columns inside microstructures. Sequential addition of reagents and samples in each microstructure is fully automated and performed according to a Bioaffy 1000 CD slow analyte spin 3-step (capture-analyte-detection) wizard method (1000-3W-005-Wash 2). Added biotinylated mAb ProtE/5 is first stopped by hydrophobic breaks. Then, centrifugal force generated by the rotation of CD drives reagent into columns which binds to streptavidin-coated particles.
iii) After column washing, reference standard and samples are then applied to activated columns.
iv) Alexa Fluor647-labeled mAb PEPilA/3 is then added to detect captured analyte and fluorescence is read by the laser. Intensity of each sample is calculated using a four logistic parameters curve to the standard curve.

In an embodiment there is therefore provided, an assay to determine potency with respect to PE-PilA using the PE antigen binding protein of the invention and the PilA antigen binding protein of the invention, wherein the assay is a sandwich ELISA assay and wherein the sandwich ELISA assay is conducted using the GYROLAB system.

In an embodiment the assay of the invention is conducted using the GYROLAB system wherein the relative potency of an UspA2 test antigen (for example SEQ ID NO: 157) and the relative potency of a Protein D antigen (for example SEQ ID NO: 139) is measured simultaneously to the relative potency of the PE-PilA antigen (for example SEQ ID NO: 122) (i.e. on the same GYROLAB CD).

As an alternative to using a conjugated enzyme as the label, other labeling is possible. For instance, other indirect labels {i.e. alternative to enzymes) can be used, but it is also possible to label the antibody by conjugation to a direct label such as a coloured particle, an electrochemically active reagent, a redox reagent, a radioactive isotope, a fluorescent label or a luminescent label. As a further alternative, the primary antibody can be conjugated to a high affinity tag such as biotin, avidin or streptavidin. An enzyme conjugated to a ligand for the tag, such as avidin, streptavidin or biotin can then be used to detect immobilized primary antibody. Any of these variations can be used within the scope and spirit of the overall invention.

In a further aspect of the invention there are provided functional antibodies for use in an in vitro potency assay.

In an embodiment the assay of the invention further comprises comparing the amount of antigen binding protein bound to the test antigen to the amount of antigen binding protein bound to a reference sample. In an embodiment the assay of the invention further comprises comparing the amount of the PilA antigen binding protein bound to the test antigen to the amount of PilA antigen binding protein bound to a reference sample (as the PilA antigen binding protein of the invention is the detection antibody). In an embodiment the reference sample is a PE-PilA fusion protein (e.g. LVL-735 of SEQ ID NO: 122) which has been tested in vivo. In an embodiment the reference sample is a PE-PilA fusion protein (e.g. LVL-735 of SEQ ID NO: 122) which has been tested in human. In an embodiment the reference sample is a PE-PilA fusion protein (e.g. LVL-735 of SEQ ID NO: 122) which has demonstrated clinical efficacy in human.

In an embodiment the assay of the invention is used to determine or measure the presence of a test antigen in its native conformation. In an embodiment the assay of the invention is used to determine or measure the potency of a test antigen. In an embodiment, the assay of the invention involves the antigen binding proteins of the invention binding to conformationally sensitive epitopes on the surface of the test antigen. The presence of such epitopes in the vaccine is expected to elicit protective antibodies in immunized patients. The assay of the invention is therefore believed to be predictive of clinical potency, and reduced binding of the neutralizing antibody reflects a subpotent vaccine. In an embodiment the assay of the invention is predictive of clinical potency.

In an embodiment the test antigen comprises Protein E and/or PilA. In an embodiment the test antigen comprises Protein E and PilA. In an embodiment, the test antigen comprises a fusion protein of Protein E and PilA. In an embodiment the fusion protein comprises a fragment of Protein E and a fragment of PilA. In an embodiment the fusion protein comprises SEQ ID NO: 55 (or sequences with at least 80% identity to SEQ ID NO: 55) and SEQ ID NO: 120 (or sequence with at least 80% identity of SEQ ID NO: 120). In an embodiment the test antigen is LVL-735 (SEQ ID NO: 122) or sequences with at least 80% identity to LVL-735 (SEQ ID NO: 122). In an embodiment the test antigen is a sequence with at least 85%, at least 90%, at least 91%, at least 92%, at least 93%, at least 94%, at least 95%, at least 96%, at least 97%, at least 98%, at least 99% or 100% identity to LVL-735 (SEQ ID NO: 122). In an embodiment the test antigen is LVL-735 (SEQ ID NO: 122). In an embodiment, the test antigen is diluted during the assay of the invention.

In a further aspect there is provided a binding assay for in vitro analysis of a Haemophilus influenzae antigen vaccine sample from a batch of final vaccine in the form in which it would be released to the public comprising the steps of:
i) permitting the H. influenzae protein antigen within the vaccine sample to interact with an antigen binding protein of the invention (optionally an antibody i.e. a monoclonal antibody) which either a) inhibits vitronectin and/or laminin binding, or b) recognises a conformational epitope in the H. influenzae antigen; then
ii) measuring the interaction between the H.influenzae antigen and antibody from step (i) wherein the binding assay is an ELISA (optionally a sandwich ELISA).

In a further aspect there is provided a binding assay for in vitro analysis of a vaccine sample comprising Protein E and/or PilA from a batch of final vaccine in the form in which it would be released to the public comprising the steps of:
i) permitting the H. influenzae protein antigen within the vaccine sample to interact with an antigen binding protein of the invention (optionally an antibody i.e. a monoclonal antibody) which either a) inhibits vitronectin and/or laminin binding, or b) recognises a conformational epitope in Protein E and/or PilA; then
ii) measuring the interaction between the H. influenzae antigen and antibody from step (i) wherein the binding assay is an ELISA (optionally a sandwich ELISA).

In an embodiment the sample is analysed in the form in which it is taken from the batch, either at full strength or after dilution. In an embodiment said vaccine sample includes H. influenzae antigen Protein E and PilA (for example the fusion protein LVL-725 of SEQ ID NO: 122) and wherein the monoclonal antibody used in step i) which recognises the H. influenzae Protein E antigen is the PE antigen binding protein of the invention. In an embodiment the conformational epitope is the epitope provided by amino acid residues I41 to Y49 and Y141 to A154 of Protein E (numbering according to SEQ ID NO: 1).

Kits

The invention further provides kits for use in the methods of the invention. There is provided a kit to (i) detect, measure the levels of, and/or measure a change in the conformation of a test antigen or (ii) determine potency of a test antigen, comprising: reagents for preparing an assay mixture, an antigen binding protein of the invention, and optionally instructions for use thereof.

In an embodiment the kits comprise all reagents and materials required in order (i) to detect, measure the levels of, and/or measure a change in conformation of a test antigen or (ii) determine potency of a test antigen. Alternatively, there is provided kits which comprise a subset of the reagents and materials required in order (i) to detect, measure the levels of, and/or measure a change in conformation of a test antigen or (ii) determine potency of a test antigen (for example wherein the kit comprises all essential buffers, reagents and consumables but does not comprise instrumentation, devices, probes etc). In an embodiment, the kit further comprises instructions for use.

The invention thus also provides a kit which is used (i) to detect, measure the levels of, and/or measure a change in conformation of a test antigen or (ii) to determine potency of a test antigen. Such kits include the antigen binding protein of the invention (e.g. a PE antigen binding protein which binds to Protein Eat one or more of amino acid residues within I41 to Y49 and Y141 to A154 of Protein E). The kit may further comprise the PilA antigen binding protein of the invention which binds to PilA at one or more of the amino acid residues within C62 to A81 of PilA.

The kit includes containers for storing reagents prior to use. Each reagent may have its own container, or several reagents may be pre-mixed and packaged together in a container.

The testing device is preferably a multi-well microtiter plate (e.g., 96 well microtiter plate), but can also be any type of receptacle such as petri dishes or plates with a plurality of wells in which an assay can be conducted. The reagents may be disposed in the wells of the testing device, although it will be appreciated that such reagents can instead be dispensed in the wells of the testing device by the end user just prior to conducting the assay. The kit may further include a set of instructions for using the kit in an assay. The kit may optionally be supplied frozen, suitable for storage at 2-8° C. or may be supplied at room temperature. In an embodiment the kit may be supplied in different components, each with different storage requirements. In an embodiment components of the kit may be supplied in lyophilized or biotinylated form and may require resuspension by the end-user prior to conducting the assay of the invention. In an embodiment the components of the kit are supplied sterile.

In an embodiment the kit requires the end user to dilute their test antigen prior to use (optionally 2-fold, optionally 10-fold, optionally 50-fold, optionally 100-fold, optionally 1000-fold, optionally 10,000-fold or greater). In an embodiment the kit further comprises a reference or internal standard which may be used to compare against the response observed with the test antigen.

The kit of the invention may further comprise an expiration date, after which the integrity of the kit can no longer be assured.

Method for Analysis

The invention further provides a method for in vitro analysis of a test antigen, comprising steps of: (i) performing the assay of the invention on a test antigen and a reference sample of known potency; and (ii) comparing the results from step (i) to determine the potency of the test antigen relative to the reference sample.

In an embodiment there is provided a method for analysing a batch of vaccine, comprising steps of: (i) assaying a test antigen taken from a batch of vaccine by the method of the invention and, if the results of step (i) indicate an acceptable relative potency, (ii) releasing further vaccines from the batch for in vivo use. In an embodiment the method of the invention is carried out in duplicate, triplicate or more. In an embodiment an acceptable relative potency will be demonstrated when the test antigen is within the specification limits of the assay, as compared to the reference sample, wherein the specification limit is set as approximately 75%-125% of the reference sample. In an embodiment an acceptable relative potency will be achieved when the ED₅₀ of the test antigen is above a threshold limit. In an embodiment an acceptable relative potency will be achieved when no statistically significant difference is observed between the data of the test antigen compared to the data of the reference sample. In an embodiment, the test antigen will fail is an acceptable relative potency is not achieved. In an embodiment, a test antigen which fails the assay of the invention will not be released to the public.

In an embodiment the test antigen will be diluted prior to or during the assay of the invention. In an embodiment the test antigen will be diluted optionally 2-fold, optionally 10-fold, optionally 50-fold, optionally 100-fold, optionally 1000-fold, optionally 10,000-fold or greater.

Embodiments of the invention are further described in the subsequent numbered paragraphs:
1. An antigen binding protein which binds to Protein E at one or more of amino acid residues within I41 to Y49 (e.g. SEQ ID NO: 133) and Y141 to A154 (e.g. SEQ ID NO: 134) of Protein E.
2. The antigen binding protein according to paragraph 1 which binds to Protein E at one or more of amino acid residues within I41 to Y49 of Protein E (e.g. SEQ ID NO: 133) and at one or more of amino acid residues within Y141 to A154 of Protein E (e.g. SEQ ID NO: 134).
3. The antigen binding protein according to paragraph 1 or paragraph 2 which binds to an epitope within or comprising amino acid residues I41 to Y49 and Y141 to A154 of Protein E (e.g. the amino acid residue of SEQ ID NO: 133 and SEQ ID NO: 134).
4. The antigen binding protein according to any of paragraphs 1-3 which binds to an epitope comprising or consisting of amino acid residues I41 to Y49 (e.g. SEQ ID NO: 133) and Y141 to A154 (e.g. SEQ ID NO: 134) of Protein E.
5. The antigen binding protein according to any of paragraphs 1-4 which binds to an epitope comprising or consisting of i) SEQ ID NO: 133 and SEQ ID NO: 134 or ii) variants of SEQ ID NO: 133 and 134, wherein said variants comprise 1, 2 or 3 amino acid modifications.
6. The antigen binding protein according to paragraphs 3-5 wherein the epitope is a conformational epitope.
7. The antigen binding protein according to paragraph 6 wherein the epitope is associated with an immunogenically active form of Protein E.
8. The antigen binding protein according to paragraph 6 or paragraph 7 wherein the epitope is associated with the native conformation of Protein E.
9. The antigen binding protein according to paragraph 8 which binds to Protein E in its native conformation with a higher specificity and/or affinity than to Protein E in a non-native conformation.
10. The antigen binding protein according to any of paragraphs 1 to 9 which inhibits laminin binding.
11. The antigen binding protein according to any of paragraphs 1 to 10 which is an antibody.
12. The antigen binding protein according to paragraph 11 which is a monoclonal antibody, optionally an IgG2a monoclonal antibody, optionally ProtE/5.
13. The antigen binding protein according to any of paragraphs 1-12 comprising: a VH region comprising a sequence at least 80% identical to the sequence of SEQ ID NO: 124; and/or a VL region comprising a sequence at least 80% identical to the sequence of SEQ ID NO: 129
14. The antigen binding protein according to paragraph 13 comprising: a VH region comprising SEQ ID NO: 124; and/or a VL region comprising SEQ ID NO: 129.
15. An antigen binding protein comprising any one ora combination of CDRs selected from CDR-H1, CDR-H2, CDR-H3 from SEQ ID NO: 124 , and/or CDR-L1, CDR-L2, CDR-L3 from SEQ ID NO: 129; or (ii) a CDR variant of (i), wherein the variant has 1, 2, or 3 amino acid modifications in each CDR, which is able to bind to Protein E at one or more of amino acid residues within I41 to Y49 and Y141 to A154 of Protein E (e.g. SEQ ID NO: 133 and SEQ ID NO: 134).
16. The antigen binding protein according to paragraph 15 comprising any one or a combination of CDRs selected from CDR-H1 (SEQ ID NO: 125), CDR-H2 (SEQ ID NO: 126) or CDR-H3 (SEQ ID NO: 127), and/or CDR-L1 (SEQ ID NO: 130), CDR-L2 (SEQ ID NO: 131) or CDR-L3 (SEQ ID NO: 132) or (ii) a CDR variant of (i), wherein the variant has 1, 2, or 3 amino acid modifications in each CDR, which is able to bind to Protein E at one or more of amino acid residues within I41 to Y49 and Y141 to A154 of Protein E (e.g. SEQ ID NO: 133 and SEQ ID NO: 134).
17. An antigen binding protein that binds to Protein E and competes for binding at one or more of amino acid residues within I41 to Y49 (e.g. SEQ ID NO: 133) and Y141 to A154 (e.g. SEQ ID NO: 134) of Protein E with reference to the antigen binding protein with a VH region comprising SEQ ID NO: 124 and a VL region comprising SEQ ID NO: 129.
18. An antigen binding protein which binds to PilA at one or more of amino acid residues within C62 to A81 (e.g. SEQ ID NO: 135) of PilA.
19. The antigen binding protein according to paragraph 18 which binds to an epitope within or comprising amino acid residues C62 to A81 of PilA (e.g. SEQ ID NO: 135).
20. The antigen binding protein according to paragraph 18 or paragraph 19 which binds to an epitope comprising or consisting of amino acid residues C62 to A81 of PilA (e.g. SEQ ID NO: 135).
21. The antigen binding protein according to any of paragraphs 18-20 which binds to an epitope comprising or consisting of i) SEQ ID NO: 135 or ii) variants of SEQ ID NO: 135 wherein said variants comprise 1, 2 or 3 amino acid modifications.
22. The antigen binding protein according to paragraphs 19-21 wherein the epitope is a conformational epitope.
23. The antigen binding protein according to paragraph 22 wherein the epitope is associated with an immunogenically active form of PilA.
24. The antigen binding protein according to paragraph 22 or paragraph 23 wherein the epitope is associated with the native conformation of PilA.
25. The antigen binding protein according to paragraph 24 which binds to PilA in its native conformation with a higher specificity and/or affinity than to PilA in a non-native conformation.
26. The antigen binding protein according to any of paragraphs 18-25 which inhibits biofilm formation.
27. The antigen binding protein according to any of paragraphs 18-26 which is an antibody.
28. The antigen binding protein according to paragraph 27 which is a monoclonal antibody, optionally an IgG2a monoclonal antibody, optionally PEPilA/3 mAb.
29. The antigen binding protein according to any of paragraphs 18-28 comprising: a VH region comprising a sequence at least 80% identical to the sequence of SEQ ID NO: 161; and/or a VL region comprising a sequence at least 80% identical to the sequence of SEQ ID NO: 163
30. The antigen binding protein according to paragraph 29 comprising: a VH region comprising SEQ ID NO: 161; and/or a VL region comprising SEQ ID NO: 163.
31. An antigen binding protein comprising any one or a combination of CDRs selected from CDR-H1, CDR-H2, CDR-H3 from SEQ ID NO: 161 , and/or CDR-L1, CDR-L2, CDR-L3 from SEQ ID NO: 163; or (ii) a CDR variant of (i), wherein the variant has 1, 2, or 3 amino acid modifications in each CDR, which is able to bind to PilA at one or more of amino acid residues within C62 to A81 of PilA (e.g. SEQ ID NO: 135).
32. The antigen binding protein according to paragraph 31 comprising any one or a combination of CDRs selected from CDR-H1 (SEQ ID NO: 164), CDR-H2 (SEQ ID NO: 165) or CDR-H3 (SEQ ID NO: 166), and/or CDR-L1 (SEQ ID NO: 167), CDR-L2 (SEQ ID NO: 168) or CDR-L3 (SEQ ID NO: 169) or (ii) a CDR variant of (i), wherein the variant has 1, 2, or 3 amino acid modifications in each CDR, which is able to bind to PilA at one or more of amino acid residues within C62 to A81 of PilA (e.g. SEQ ID NO: 135).
33. An antigen binding protein that binds to Protein E and competes for binding at one or more of amino acid residues within C62 to A81 (e.g. SEQ ID NO: 135) of PilA with reference to the antigen binding protein with a VH region comprising SEQ ID NO: 161 and a VL region comprising SEQ ID NO: 163.
34. An immunogenic composition comprising the antigen binding protein of any of paragraphs 1-17 and/or the antigen binding protein of any of paragraphs 18-33.
35. A vaccine comprising the antigen binding protein of any of paragraphs 1-17 and/or the antigen binding protein of any of paragraphs 18-33.
36. The vaccine according to paragraph 35 further comprising an adjuvant.
37. An antigen binding protein as defined in any of paragraphs 1-33, or an immunogenic composition as defined in paragraph 34, or a vaccine as defined in paragraph 35 or paragraph 36, for use in therapy.
38. An antigen binding protein as defined in any of paragraphs 1-33, or an immunogenic composition as defined in paragraph 34 or a vaccine as defined in paragraph 35 or paragraph 36, for use in treating or preventing an infection, disease or condition caused by H. influenzae.
39. An antigen binding protein as defined in any of paragraphs 1-33, or an immunogenic composition as defined in paragraph 34 or a vaccine as defined in paragraph 35 or paragraph 36, for use in treating or preventing an infection, disease or condition which is otitis media, pneumonia and/or acute exacerbations of chronic obstructive pulmonary disease (AECOPD).
40. An antigen binding protein as defined in any of paragraphs 1-33, or an immunogenic composition as defined in paragraph 34 or a vaccine as defined in paragraph 35 or paragraph 36, for use in treating or preventing an infection, disease or condition caused by H. influenzae, in a mammal, particularly a human.
41. An antigen binding protein as defined in any of paragraphs 1-33, or an immunogenic composition as defined in paragraph 34 or a vaccine as defined in paragraph 35 or paragraph 36, for use in the treatment or prevention of acute otitis media, pneumonia and/or acute exacerbations of chronic obstructive pulmonary disease (AECOPD).
42. An antigen binding protein as defined in any of paragraphs 1-33, or an immunogenic composition as defined in paragraph 34 or a vaccine as defined in paragraph 35 or paragraph 36, for use in the manufacture of a medicament for the treatment or prevention of an infection, disease or condition caused by H. influenzae.
43. An antigen binding protein as defined in any of paragraphs 1-33, or an immunogenic composition as defined in paragraph 34 or a vaccine as defined in paragraph 35 or paragraph 36, for use in the manufacture of a medicament for the treatment or prevention of pneumonia, otitis media and/or acute exacerbations of chronic obstructive pulmonary disease AECOPD.
44. A method of treatment or prevention of an infection, disease or condition caused by H. influenzae, in a subject in need thereof comprising administering to said subject a therapeutically effective amount of an antigen binding protein as defined in any of paragraphs 1-33, or an immunogenic composition as defined in paragraph 34 or a vaccine as defined in paragraph 35 or paragraph 36.
45. A method of treatment or prevention of acute exacerbations of chronic obstructive pulmonary disease (AECOPD), pneumonia and/or otitis media in a subject in need thereof comprising administering to said subject a therapeutically effective amount of an antigen binding protein as defined in any of paragraphs 1-33, or an immunogenic composition as defined in paragraph 34 or a vaccine as defined in paragraph 35 or paragraph 36.
46. The use of an antigen binding protein according to paragraphs 1-17 in the detection of, or measurement of a change in, the conformation of Protein E.
47. The use of an antigen binding protein according to paragraphs 18-33 in the detection of, or measurement of a change in, the conformation of PilA.
48. The use of both the antigen binding protein according to paragraphs 1-17 and the antigen binding protein according to paragraphs 18-33 in the detection of, or measurement of a change in, the conformation of a test antigen, optionally a PE-PilA fusion protein, optionally the PE-PilA fusion protein of SEQ ID NO: 122.
49. An assay comprising exposing a sample of a test antigen to an antigen binding protein according to any of paragraphs 1-33 and measuring the amount of antigen binding protein bound to the test antigen.
50. The assay of any of paragraphs 49 which is an in vitro assay.
51. The assay of any of paragraphs 49-50, wherein the assay is an ELISA, optionally a sandwich ELISA.
52. The assay of any of paragraphs 49-51 wherein the assay is a sandwich ELISA using the antigen binding protein according to paragraphs 1-17 and the antigen binding protein according to paragraph 18-33.
53. The assay of paragraph 52 wherein the antigen binding protein according to paragraphs 1-17 is the capture antibody.
54. The assay of paragraph 52 or paragraph 53 wherein the antigen binding protein according to paragraphs 18-33 is the detection antibody.
55. The assay of paragraph 54 wherein the detection antibody is biotinylated.
56. The assay of paragraphs 54-55, wherein the detection antibody is labeled with an enzyme wherein the amount of test antigen bound to the detection antibody is determined by measuring the conversion of a substrate into a detectable product by said enzyme.
57. The assay of paragraph 56, wherein the detection antibody is labeled with an enzyme that is conjugated to streptavidin.
58. The assay of paragraph 57 wherein the enzyme that is conjugated to streptavidin is peroxidase.
59. The assay of paragraphs 56-58 wherein a substrate is utilised which, upon interaction with peroxidase, causes a detectable change, optionally a change in absorbance or fluorescence.
60. The assay of paragraph 59 wherein the substrate is o-phenylenediamine dihydrochloride.
61. The assay of any of paragraphs 49-60 further comprising comparing the amount of antigen binding protein bound to the test antigen to the amount of antigen binding protein bound to a reference sample.
62. The assay of any of paragraphs 49-61 to determine or measure the presence of a test antigen in its native conformation.
63. The assay of any of paragraph 49-62 to determine or measure the potency of a test antigen.
64. The assay of any of paragraphs 49-63 wherein the test antigen comprises Protein E and/or PilA.
65. The assay of any of paragraphs 49-64 wherein the test antigen comprises a fusion protein of Protein E and PilA.
66. The assay of any of paragraphs 49-65 wherein the test antigen is LVL-735 (SEQ ID NO: 122) or sequences with at least 80% identity to LVL-735 (SEQ ID NO: 122).
67. The assay according to any of claims 49-66 wherein the assay is performed using the GYROLAB system.
68. A kit to (i) detect, measure the levels of, and/or measure a change in the conformation of a test antigen or (ii) determine potency of a test antigen, comprising: reagents for preparing an assay mixture, an antigen binding protein according to any of paragraphs 1-33, and optionally instructions for use thereof.
69. A method for in vitro analysis of a test antigen, comprising steps of: (i) performing the assay of any of paragraphs 49-67 on a test antigen and a reference sample of known potency; and (ii) comparing the results from step (i) to determine the potency of the test antigen relative to the reference sample.
70. A method for analysing a batch of vaccine, comprising steps of: (i) assaying a test antigen taken from a batch of vaccine by the method of paragraph 69; and, if the results of step (i) indicate an acceptable relative potency, (ii) releasing vaccine from the batch for in vivo use.

Embodiments of the invention are yet further described in the subsequent numbered paragraphs:
1. An antigen binding protein which binds to Protein E at one or more of amino acid residues within I41 to Y49 (e.g. SEQ ID NO: 133) and Y141 to A154 (e.g. SEQ ID NO: 134) of Protein E.
2. The antigen binding protein according to paragprah 1 which binds to Protein E in its native conformation with a higher specificity and/or affinity than to Protein E in a non-native conformation.
3. The antigen binding protein according to paragrpah 1 or paragraph 2 which inhibits laminin binding.
4. The antigen binding protein according to any of paragraphs 1-3 comprising: a VH region comprising a sequence at least 80% identical to the sequence of SEQ ID NO: 124; and/or a VL region comprising a sequence at least 80% identical to the sequence of SEQ ID NO: 129.
5. An antigen binding protein which binds to PilA at one or more of amino acid residues within C62 to A81 (e.g. SEQ ID NO: 135) of PilA.
6. The antigen binding protein according to paragraph 5 which binds to PilA in its native conformation with a higher specificity and/or affinity than to PilA in a non-native conformation.
7. The antigen binding protein according to paragraph 5 or paragraph 6 which inhibits biofilm formation.
8. The antigen binding protein according to any of paragraphs 5-7 comprising: a VH region comprising a sequence at least 80% identical to the sequence of SEQ ID NO: 161; and/or a VL region comprising a sequence at least 80% identical to the sequence of SEQ ID NO: 163.
9. An immunogenic composition comprising the antigen binding protein of any of paragraphs 1-4 and/or the antigen binding protein of any of paragraphs 5-8.
10. A vaccine comprising the antigen binding protein of any paragraphs 1-4 and/or the antigen binding protein of any of paragraph 5-8, optionally further comprising an adjuvant.
11. An antigen binding protein as defined in any of paragraphs 1-8, or an immunogenic composition as defined in paragraph 9 or a vaccine as defined in paragraph 10, for use in preventing or treating an infection, disease or condition caused by H. influenzae, optionally otitis media, pneumonia and/or acute exacerbations of chronic obstructive pulmonary disease (AECOPD).
12. The use of both the antigen binding protein according to paragraphs 1-4 and the antigen binding protein according to paragraphs 5-8 in the detection of, or measurement of a change in, the conformation of a test antigen optionally a PE-PilA fusion protein, optionally the PE-PilA fusion protein of SEQ ID NO: 122.
13. An assay comprising exposing a sample of a test antigen to an antigen binding protein according to any of paragraphs 1-8 and measuring the amount of antigen binding protein bound to the test antigen, optionally wherein the assay is an in vitro assay, optionally an ELISA, optionally a sandwich ELISA.
14. The assay of any of claim 13 further comprising comparing the amount of antigen binding protein bound to the test antigen to the amount of antigen binding protein bound to a reference sample, optionally wherein the assay is to determine or measure the presence of a test antigen in its native conformation, optionally wherein the assay is used to determine or measure the potency of a test antigen.
15. The assay of claim 13 or claim 14 wherein the test antigen comprises Protein E and/or PilA, optionally wherein the test antigen comprises a fusion protein of Protein E and PilA, optionally LVL-735 (SEQ ID NO: 122) or sequences with at least 80% identity to LVL-735 (SEQ ID NO: 122).
16. A method for in vitro analysis of a test antigen, comprising steps of: (i) performing the assay of any of claims 13-15 on a test antigen and a reference sample of known potency; and (ii) comparing the results from step (i) to determine the potency of the test antigen relative to the reference sample

In order that this invention may be better understood, the following examples are set forth. These examples are for purposes of illustration only and are not to be construed as limiting the scope of the invention in any manner.

### EXAMPLES

### Example 1

### Evaluation of Anti-PE and Anti-PilA mAbs in a Biofilm Inhibition Assay

After overnight incubation at 37° C. with 5% CO₂ on chocolate agar plate, bacteria were inoculated into preheated Brain Heart infusion (BHi) medium containing additive. The optical density at 490 nm was adjusted to 0.65 and after a 1/6 dilution, the culture was incubated for 3 hours at 37° C. with 5% CO₂ under static condition. Then the culture was diluted to have 4.10⁴ CFU/200 μl/well.

After an incubation of 1 hour at 37° C., serial two-fold serial dilutions of mAbs (covering concentrations from 400 μg/ml to 6.25 μg/ml) were added into the wells. The mix was incubated for 4 hours at 37° C. with 5% CO₂. After washing, 200 μl of fresh supplemented BHi were added and plates were incubated for 16 hours at 37° C. with 5% CO₂. After washing, bacteria were colored with a live and dead staining for 15 minutes in the darkness. After washing, fixing and drying, fluorescence was captured using the Axiovision system.

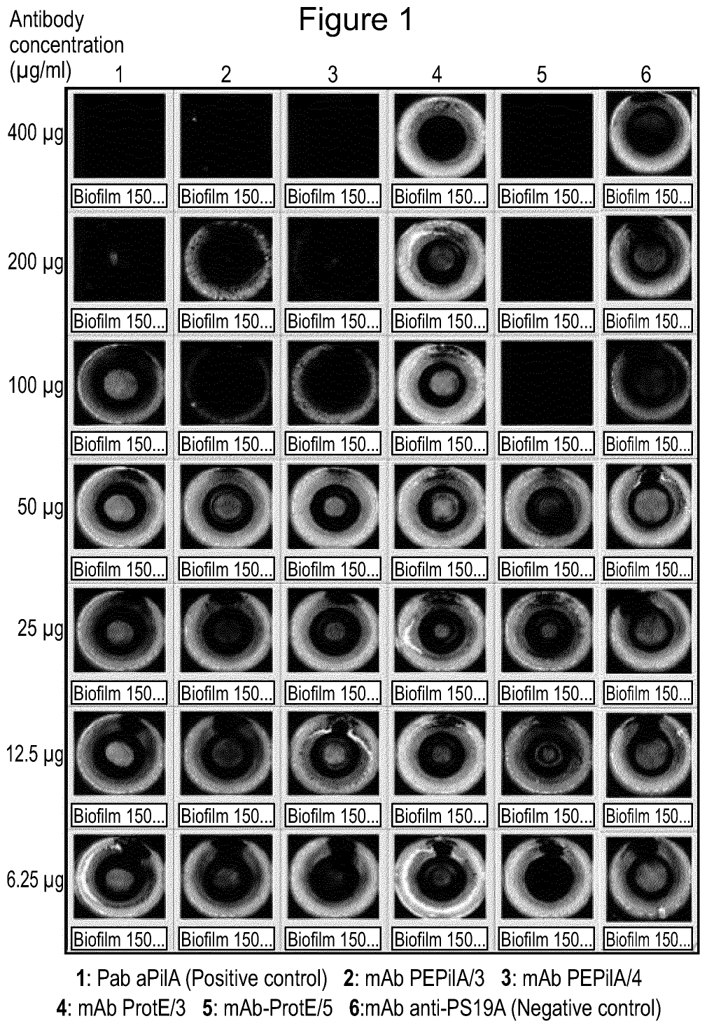

Both anti-PilA mAbs (PEPilA/3 and PEPilA/4), the anti-PE ProtE/5 mAb and a rabbit anti-PilA serum used as a positive control were shown able to prevent biofilm formation (FIG. 1).

No inhibition of biofilm formation was observed with anti-PE ProtE/3 and the negative control anti-PS19A from Streptococcus pneumoniae mAbs.

### Example 2

### ECM Binding Assays

Objectives: To analyse interactions between Protein E, PilA and PE-PilA with Vitronectin, Laminin and Fibronectin and to see if monoclonal Antibodies ProtE/5 mAb (PE) and PEPilA/3 mAb (PilA) interfere with these interactions.

Binding of Protein E and PilA and PE-PilA to ECM proteins was analysed using Biacore technology (CM-5 sensor chips) according to manufacturer's recommendations.

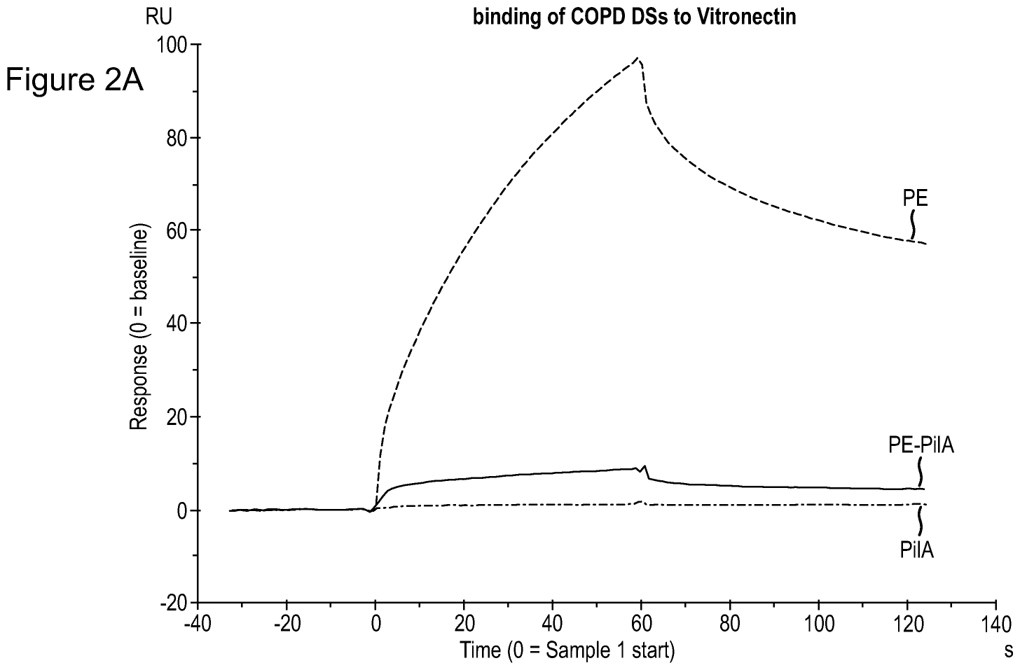

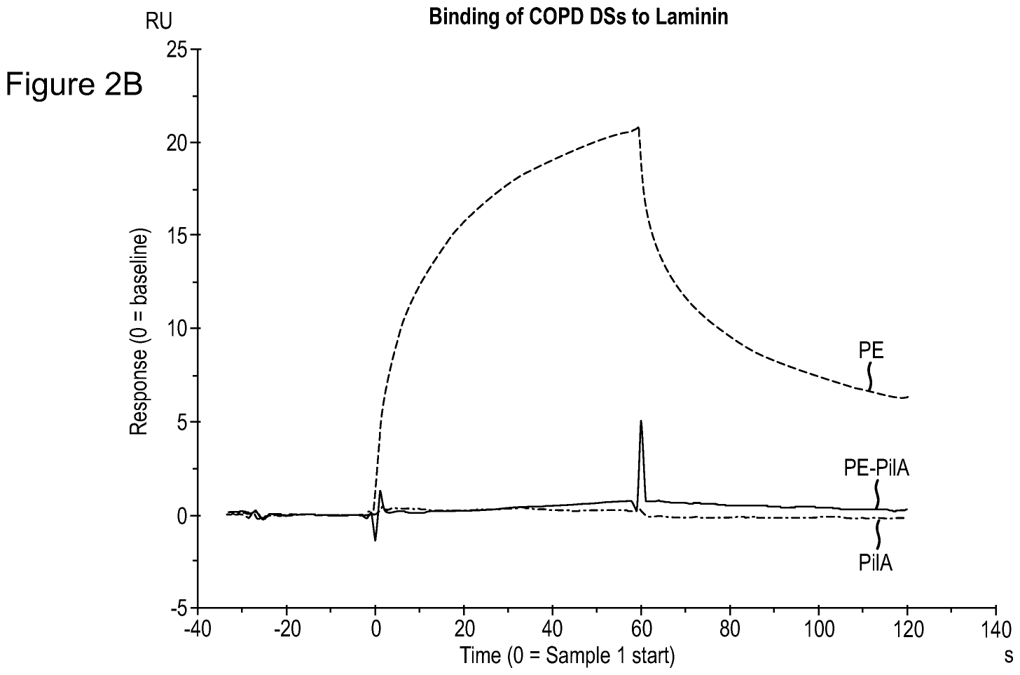

Results: PE was demonstrated to bind to Vitronectin and Laminin (FIG. 2) but not Fibrinogen (data not shown). Protein E complexes with Vitronectin were more stable than Protein E-Laminin complexes. No binding to Victronectin, Laminin or Fibrinogen (data not shown) was observed with either PilA or the PE-PilA fusion protein LVL-735 (SEQ ID NO: 122).

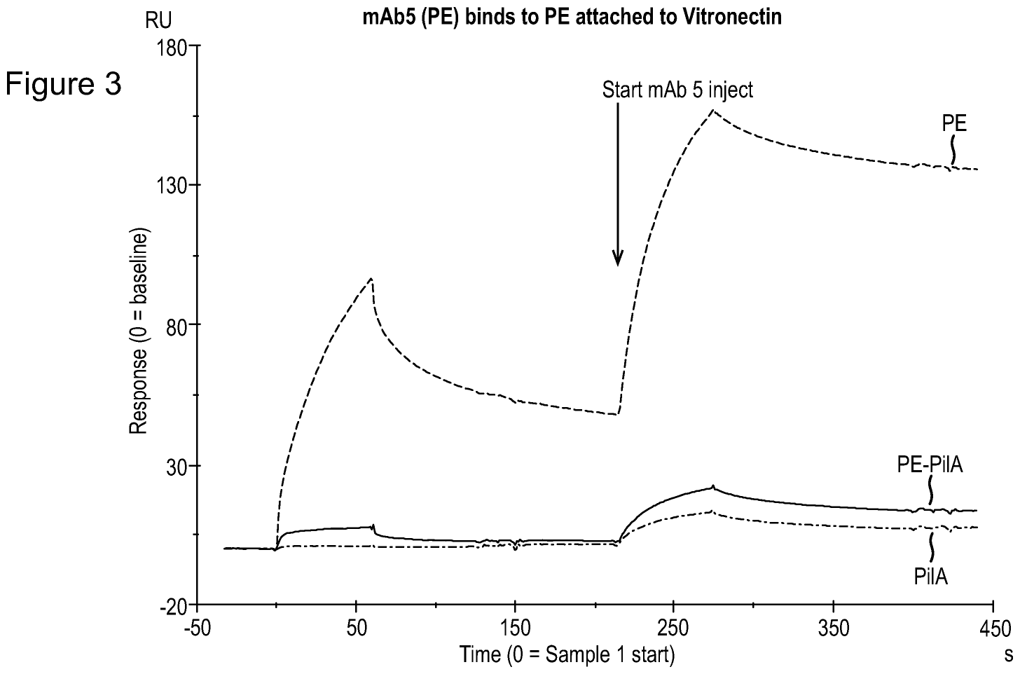

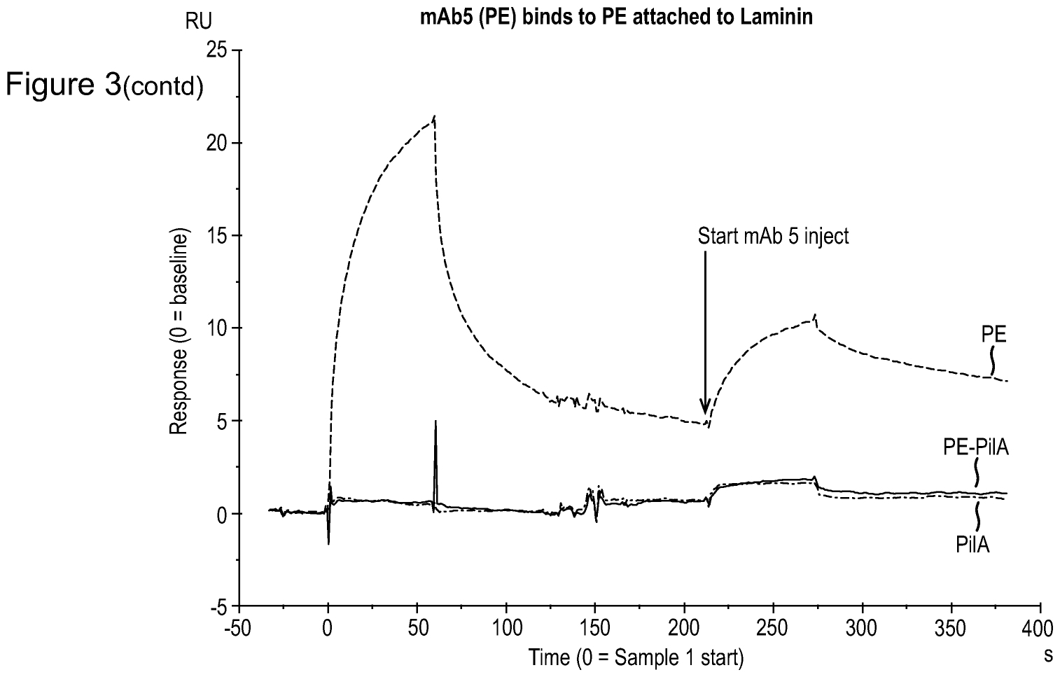

Protein E mAb5 (PE) binds to Protein E when attached either to Vitronectin or to Laminin (FIG. 3). This data suggests that, the PE dimer (i.e. Protein E attached to either VN or LM) still exposes an epitope to allow for mAb5 binding, explaining how in particular LM-attached PE can still bind mAb5.

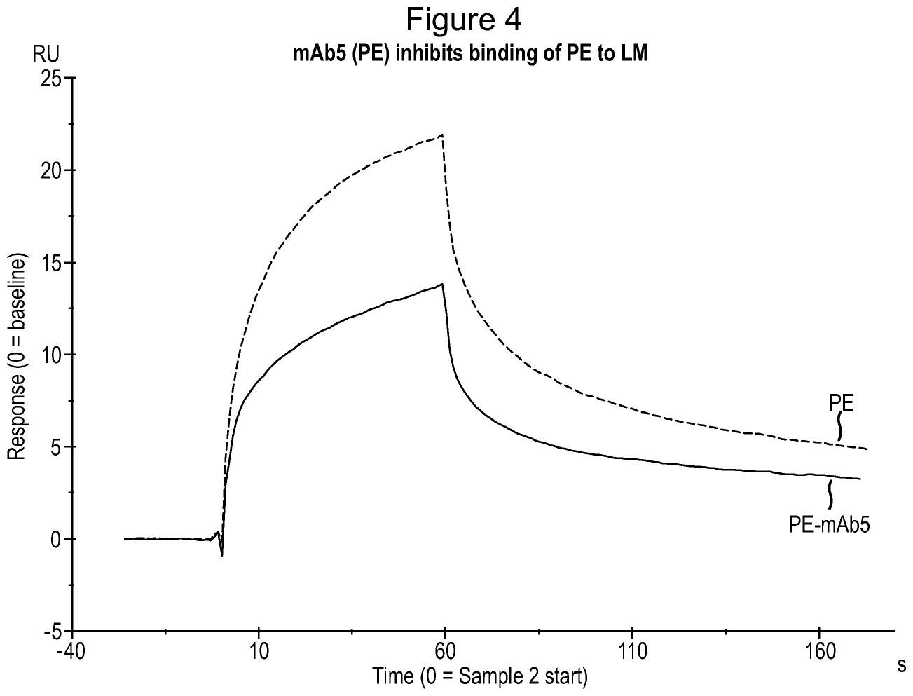

Finally, the ProtE/5 mAb successfully demonstrated inhibition of laminin binding (FIG. 4).

### Example 3

### Epitope Mapping

Materials

Deuterium oxide (99.9% D atoms), sodium deuteroxide, deuterium chloride, acetonitrile and Glu-fibrinogen peptide (GFP) were all purchased from Sigma-Aldrich and used without further purification. Poroszyme immobilised pepsin column was purchased from Thermo-Fisher.

Methods

Sample Preparation for HDX-MS Analyses.

1. The antibody/antigen complex was formed by adding 378 pmoles of PE-PilA fusion (LVL-735) to either the PE/5 antibody or the PE-PilA/3 using a molar ratio PE-PilA/mAb of 1:1 and incubated for 30 min at 25° C.

2. The labeling was initiated by adding deuterated PBS buffer (pD of 7.3), reaching a deuterium excess of 92.3% for the experiment with the mAb PE/5 and 90.8% for the experiment with the mAb PE-PilA/3, at 25° C. Over the time course of the experiment (ranging from 30 sec to 24 hours), 30 μL of the sample were removed and quenched with the same volume of an ice-cold quenching buffer (7M urea, 400 mM GuCl, 800 mM TCEP, 0.1% F.A., pH 2.4) to dissociate the antibody/antigen complex and to lower the pH to 2.4. The quenched aliquots were immediately frozen in liquid nitrogen and stored at −80° C. for less than 24 h.

A control experiment without antibody was prepared using the same conditions previously described (PBS was used instead of the antibody preparation). Labelled samples were immediately flash frozen in liquid nitrogen and stored at −80° C. for less than 24 h.

Local HDX-MS Analyses

Data processing Labelled samples were thawed rapidly to 0° C. and injected into a Waters nanoACQUITY UPLC with HDX Technology. The injector, switching valve, columns, solvents and all associated tubings were at 0° C. to limit back-exchange. For local HDX-MS, protein samples were on-line digested for 2.5 min at 20° C. with a flow rate of 200 μL/min using a Poroszyme Immobilized Pepsin Cartridge (2.1 mm×20 mm, Thermo-Fisher) equilibrated with 100% buffer A (2% acetonitrile, 0.1% formic acid in water). The generated peptides were immediately trapped, concentrated and desalted using a VanGuard BEH Pre-column (1.7 μm, 2.1×5 mm, Waters). The 2.5 min digestion and desalting step allows deuterons located at fast exchanging sites (i.e. side chains and amino/carboxy terminus) to be replaced with hydrogens. Peptides were then separated on an ACQUITY UPLC BEH C18 reverse phase column (1.7 μm, 1.0×100 mm, Waters) with a linear gradient from 10 to 40% buffer B (2% water, 0.1% formic acid in acetonitrile) over 6.8 min at 40 μL/min.

Mass spectra acquisition Mass spectra were acquired in resolution mode (m/z 300-2000) on a Waters SynaptG2 mass spectrometer equipped with a standard ESI source. The mass spectrometer SynaptG2 is calibrated before each analysis with a Caesium iodide solution (2 mg\mL in 50% isopropanol) infused through the reference probe of the ESI source. Mass accuracy was ensured by continuously infusing a GFP solution (600 fmol/μL in 50% acetonitrile, 0.1% formic acid) through the reference probe of the ESI source. The identity of each peptide was confirmed by MSE analyses. MSE was directly performed by a succession of low (6 V) and high collision (25 V) energies in the transfer region of the mass spectrometer. All fragmentations were performed using argon as collision gas. Data were processed using Protein Lynx Global Server 3.0.1 (Waters) and each fragmentation spectrum was manually inspected to confirm the assignment. The DynamX 3.0 software (Waters) was used to select the peptides considered for the analysis and to extract the centroid mass of each of them, and for each charge state, as a function of the labeling time. Only the peptic peptides present in at least four over five repeated digestions of the unlabeled proteins were considered for the analysis.

Synapt G2 Settings:

Source: ES+

Capillary: 3000 V

Sample Cone: 25 V

Extraction Cone: 4 V

Source Temperature: 80° C.

Cone gas: 20 L/h

The epitope mapping of the PE-PilA protein with both the PE/5 and the PE-PilA/3 antibody was performed using the Waters nanoACQUITY UPLC with HDX Technology and DynamX software.

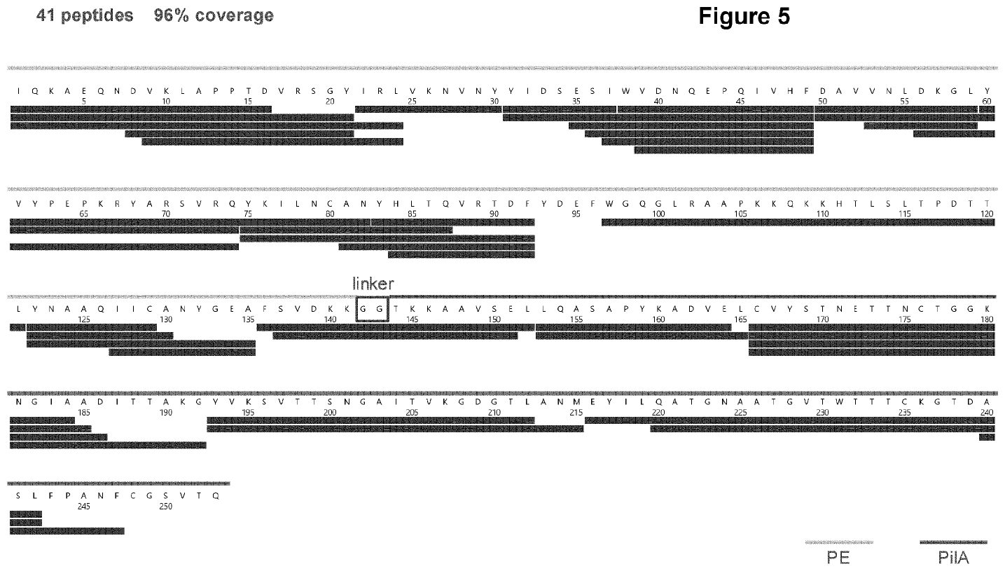

41 pepsin peptides, corresponding to 96% of the PE-PilA sequence were considered for this analysis (FIG. 5).

ProtE/5 mAb

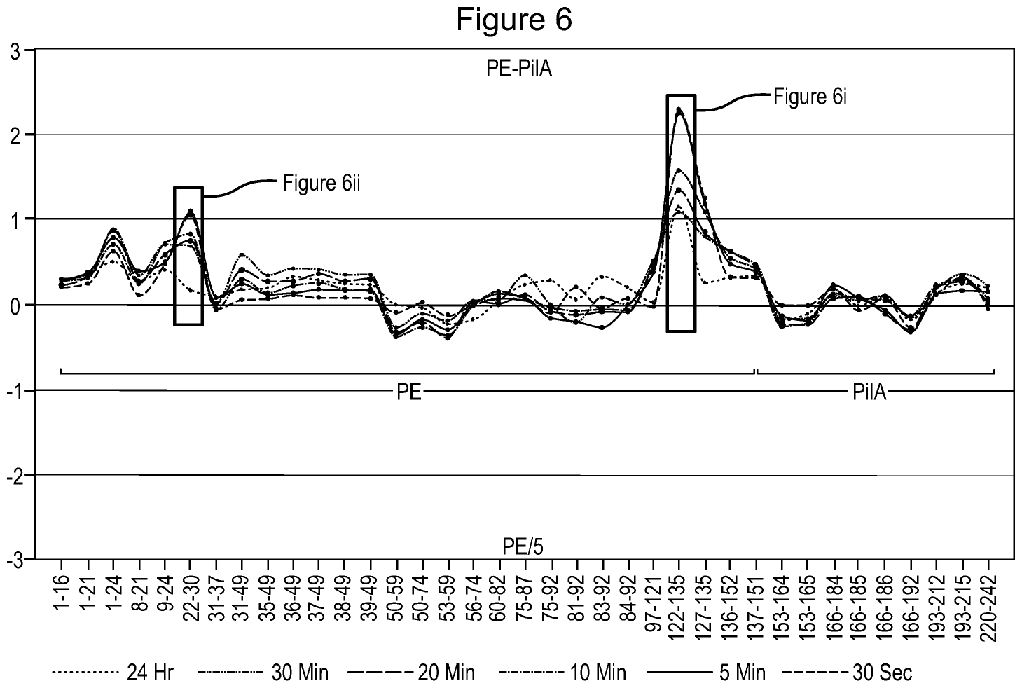

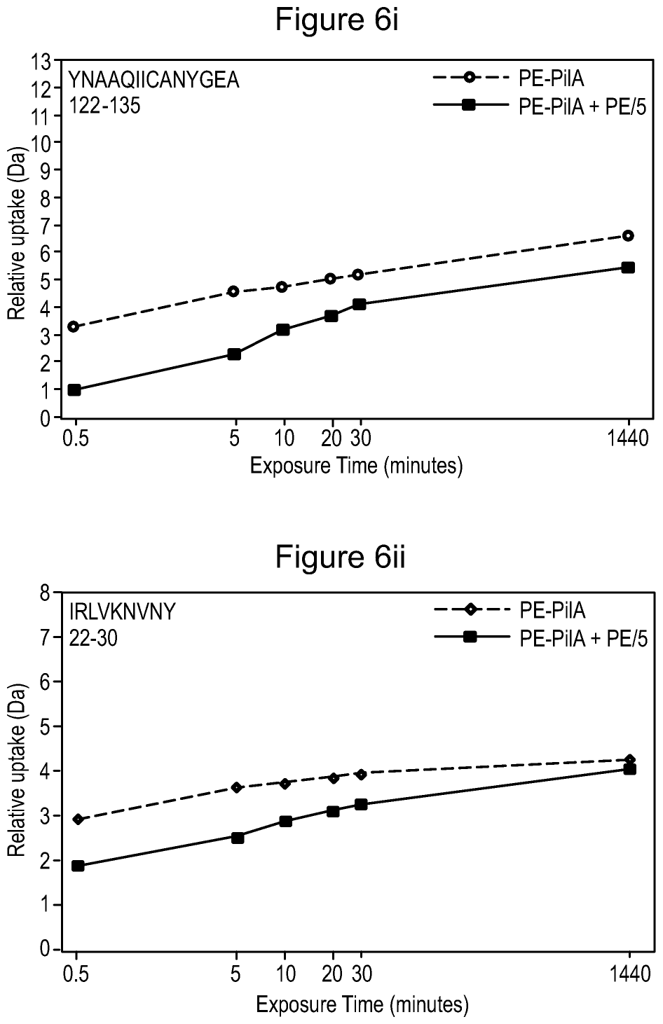

The difference in deuterium incorporation of these 41 peptides generated from the antigen (PE-PilA) alone or bound to the PE/5 mAb is reported in FIG. 6. The difference was considered significant when the averaged value of deuterium incorporation is superior to 1 Da.

Peptides 22-30 (IRLVKNVNY) and 122-135 (YNAAQIICANYGEA) showed a significant difference in deuterium uptake in presence of the mAb.

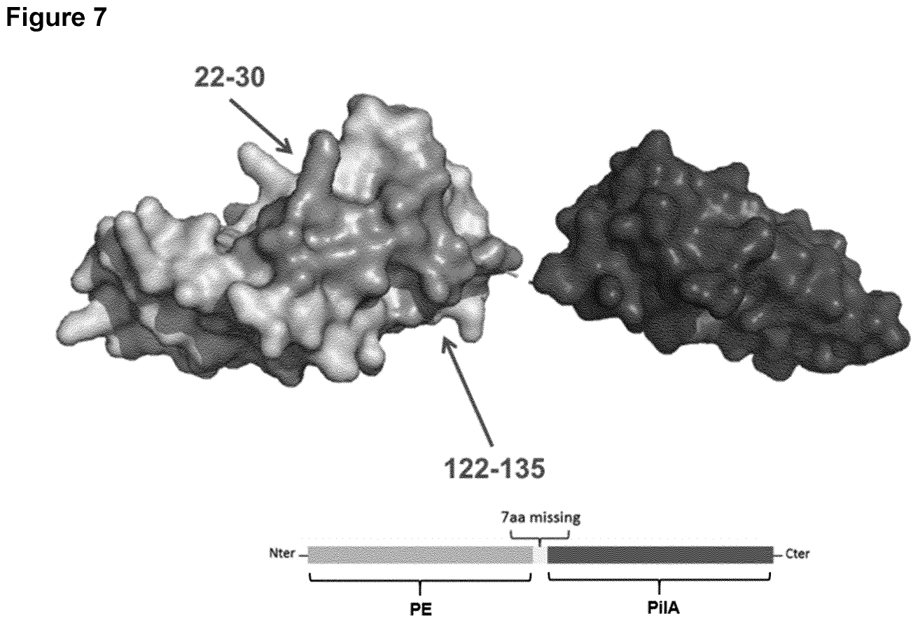

Peptides 22-30 (IRLVKNVNY) and 122-135 (YNAAQIICANYGEA) were subsequently mapped onto the 3D structure of the PE-PilA fusion protein LVL-735 (SEQ ID NO: 122). As can be observed in FIG. 7, both regions (i.e. 22-30 and 122-135) are surface exposed and structurally close.

PE-PilA/3 mAb

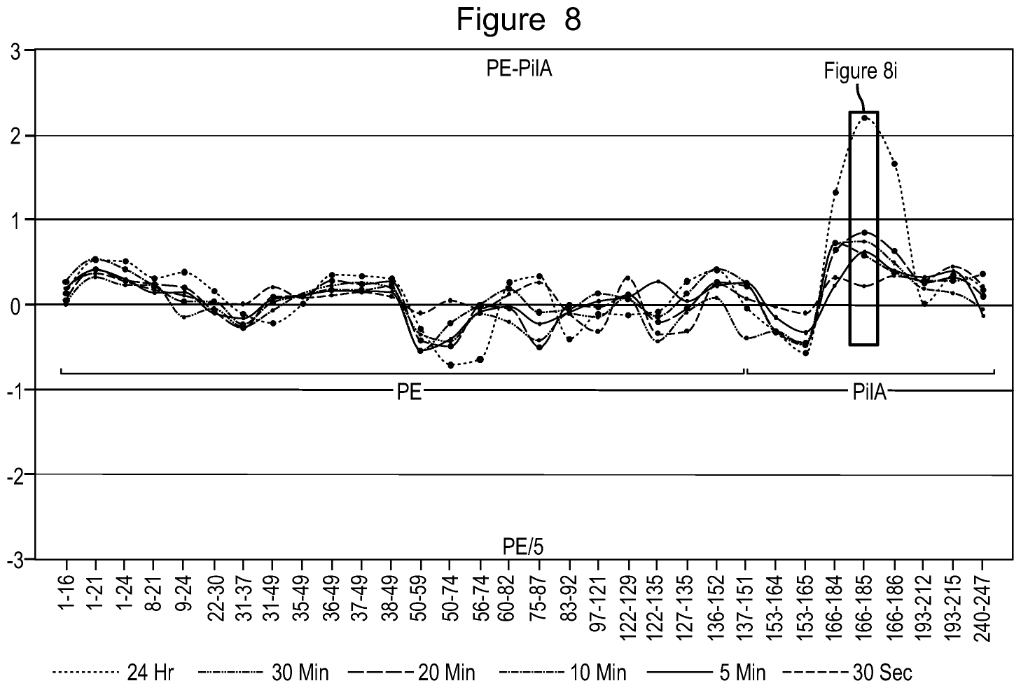

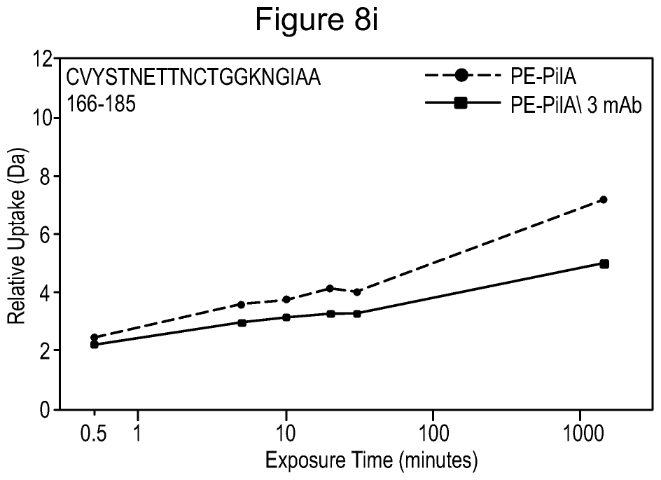

The difference in deuterium incorporation of these 41 peptides generated from the antigen (PE-PilA) alone or bound to the PE-PilA/3 mAb is reported in FIG. 8. The difference was considered significant when the averaged value of deuterium incorporation is superior to 1 Da.

Peptide 166-185 (CVYSTNETTNCTGGKNGIAA) showed a significant difference in deuterium uptake in presence of the mAb.

Peptide 166-185 was also shown to be surface exposed on the 3D PE-PilA fusion structure (see FIG. 9)

### Example 4

### Sequencing of the Anti-PE Monoclonal Antibody (mAb ProtE/5)

The nucleotide and protein sequences for the Variable Heavy and Variable Light chains of the ProtE/5 mAb were determined using the following methodology.

Aim: To obtain the nucleic and amino acid sequence of hybridoma-secreted antibody of ProtE/5 clone. The whole procedure aimed to sequence exclusively the variable regions of the light and heavy antibody chains (VL and VH). The sequencing strategy was designed to also obtain the sequence of a small region of the constant region (˜50-60 bp) for confirmation of the antibody class/subtype

Methods: The whole procedure can be summarized as follows:
1. Thawing and growth of hybridoma cell clone
2. RNA extraction
3. cDNA generation by retro-transcription
4. 3′ polyA tailing
5. 5′ Rapid Amplification of cDNA Ends (RACE) PCR
6. Cloning into commercial plasmid (TOPO PCR cloning)
7. Colony picking, bacterial growth and plasmid extraction
8. Sanger sequencing

Briefly, hybridoma cells were thawed and grown for 10 days;

Thawing of cells: 15 ml Falcon tubes were prepared containing 10 ml of warm DMEM medium. Cells were thawed by placing the cryotube rapidly in a 37° C. water bath. Cells were transferred into the Falcon tube and centrifuged for 10 minutes at 1000 rpm. The supernatant was carefully poured away, and the cells were resuspended with 10 ml of warm D-MEM and re-centrifuged for 10 minutes at 1000 rpm. In the meanwhile, 1 ml of thawing medium was transferred into each well of the first row of a 24-well plate.

The supernatant was again poured away, and the cells were resuspended with 1 ml of warm thawing media. The resuspension was transferred into the first well, mixed by gentle pipetting and then 1 ml was transferred into the near well. This 1:1 dilution was continued until the last well. 1 ml of media was added to each well, to reach 2 ml of cell culture in each well.

The plate was placed in an incubator at 37° C. with a 5% CO2 atmosphere. After few days, the cells were transferred from the well where they are not fully convergent into a T25 flask for adherent cells adding fresh thawing media to 10 ml total volume.

Cell Sub Culturing Protocol

Cells were recovered by shaking gently the T25 flask and pouring the resuspension into a 50 ml Falcon tube. The tube was then centrifuged for 10 minutes at 1000 rpm, and the pellet resuspend in warm D-MEM. This process was repeated although the second resuspension step was conducted in 50 ml of warm growing medium. The cells were then transferred into a T75 flask.

RNA was then extracted (4 samples of cells, each containing 7×10⁶ cells) using the Qiagen RNeasy Mini kit (according to manufacturer's instructions) followed by cDNA generation by retro transcription of 4.5 μg RNA. Retro transcription was performed using SuperScript IV first-strand synthesis system (Invitrogen) and a set of oligos specific for either the light chain or heavy chain amplification:

3′ polyA tailing was performed using between 680 and 200 ng of cDNA and Terminal Deoxynucleotidyl Transferase (ThermoScientific) and dATP (Invitrogen). This generated (after column purification) 400-800 ng of polyA cDNA, following which 5′ rapid amplification of cDNA ends (RACE) PCR was performed using either Q5 Hot Start polymerase (NEB) or Platinum SuperFi polymerase (Invitrogen), and a set of oligos specific for either the light chain or heavy chain amplification:

Cloning into commercial plasmid (and transformation) was performed using ZeroBlunt TOPO PCR Kits according to manufacturer's instructions (Invitrogen). Colonies were then picked, and plasmids extracted using the Qiaprep Miniprep Kit (Qiagen) and Sanger Sequences was performed.

100 ng of plasmid was used.

QIAquick Gel Extraction Kit and MinElute PCR Purification Kit (Qiagen) were used for the DNA purification steps.

Upon analysis, the sequences obtained for every tested clone share the following regions organization;
polyT sequence
5′ UTR (UnTranslated Region)+leader
Full variable heavy (VH) domain
Short region of CH1 domain
NGS adapters (5′/3′) [needed only if DNA submitted to NGS sequencing]

Using a sequence analysis software (DNASTAR LaserGene 12), everything was discarded but the antibody genes. Upon alignment, all sequences show complete homology.

Variable Heavy Chain Analysis (CDR Reqions
underlined)
DNA
(SEQ ID NO: 123)
CAGATCCAGTTGGTGCAGTCTGGACCTGAGCTGAAGAAGCCTGGAGAGAC
AGTCAAGATCTCCTGCAAGGCTTCTGGGTATACCTTCACAAACTATGGAA
TGAACTGGGTGAAGCAGGCTCCAGGAAAGGGTTTAAAGTGGATGGGCTGG
ATAAACACCTACACTGGAGAGCCAACATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGGGGAGGG
TACCCCTCCTCGCGGGCGCCCCCTTACTGGGGCCAAGGGACTCTGGTCAC
TGTCTCTGCAG
Protein
(SEQ ID NO: 124)
QIQLVQSGPELKKPGETVKISCKASGYTFTNYGMNWVKQAPGKGLKWMGW
INTYTGEPTYADDFKGRFAFSLETSASTAYLQINNLKNEDTATYFCARGG
YPSSRAPPYWGQGTLVTVSA
CDR-H1
(SEQ ID NO: 125)
NYGMN
CDR-H2
(SEQ ID NO: 126)
WINTYTGEPTYADDFKG
CDR-H3
(SEQ ID NO: 127)
GGYPSSRAPPY
Variable Liqht Chain Analysis (CDR Reqions
underlined)
DNA
(SEQ ID NO: 128)
GACATTGTGATGACCCAGTCTCACAAATTCATGTCCACATCAGTAGGAGA
CAGGGTCAGCATCACCTGCAAGGCCAGTCAGGATGTGGGTACTGCTGTAG
CCTGGTATCAACAGAAACCAGGGCAATCTCCTAAACTACTGATTTACTGG
GCATCCACCCGGCACACTGGAGTCCCTGATCGCTTCACAGGCAGTGGATC
TGGGACAGATTTCACTCTCACCATTAGCAATGTGCAGTCTGAAGACTTGG
CAGATTATTTCTGTCAGCAATATAGCAGCTATCCATTCACGTTCGGCTCG
GGGACAAAGTTGGAAATAAAA
Protein
(SEQ ID NO: 129)
DIVMTQSHKFMSTSVGDRVSITCKASQDVGTAVAWYQQKPGQSPKLLIYW
ASTRHTGVPDRFTGSGSGTDFTLTISNVQSEDLADYFCQQYSSYPFTFGS
GTKLEIK
CDR-L1
(SEQ ID NO: 130)
KASQDVGTAVA
CDR-L2
(SEQ ID NO: 131)
WASTRHT
CDR-L3
(SEQ ID NO: 132)
QQYSSYPFT

For both the heavy and light chain variable regions multiple chains were identified during sequence analysis. However only two (i.e. the variable heavy chain and the variable light chain shown above) were free of structural abnormalities e.g. abnormal cysteine content, early stop codons etc. Sequencing was also able to confirm that the ProtE/5 mAb is an IgG2A mAb.

Sequencing was conduced as described above for the PilA antigen binding protein of the invention (i.e. PEPilA/3 mAb). The sequences are as follows (CDR regions are underlined).

VH PEPilA/3
>DNA
(SEQ ID NO: 160)
CAGGGTCAGCTGCAGCAGTCTGGACCTGAGCTGGTGAAGCCTGGGGCTT
CAGTGAAGATGTCCTGCAAGGCTTCTGGATACACATTCACTGACTATGT
TATAAGCTGGGTGAAGCAGAGAATTGGACAGGGCCTTGAGTGGATTGGA
GAGATTCATCCTGGAAGTGGTAGTATTCACTACAATGAGAAGTTCAAGG
GCAAGGCCACACTGACTGCAGACAAATCCTCCAACACAGCCTACATGCA
GCTCAGCAGCCTGACATCTGAGGACTCTGCGGTCTATTTCTGTGCAAGA
AGGGGGTTACGACGTCCCTGGTTTGCTTACTGGGGCCAAGGGACTCTGG
TCACTGTCTCTGCAG
>Protein
(SEQ ID NO: 161)
QGQLQQSGPELVKPGASVKMSCKASGYTFTDYVISWVKQRIGQGLEWIG
EIHPGSGSIHYNEKFKGKATLTADKSSNTAYMQLSSLTSEDSAVYFCAR
RGLRRPWFAYWGQGTLVTVSA
VL PEPilA/3
>DNA
(SEQ ID NO: 162)
GACATTGTGCTGACACAGTCTCCTGCTTCCTTAGCTGTATCTCTGGGGC
AGAGGGCCACCATCTCATGCAGGGCCAGCAAAAGTGTCAGTACATCTGG
CTATAGTTATATGCACTGGTTCCAACAGAAACCAGGACAGCCACCCAAA
CTCCTCATCTATCTTGCATCCAACCTAGAATCTGGGGTCCCTGCCAGGT
TCAGTGGCAGTGGGTCTGGGACAGACTTCACCCTCAACATCCATCCTGT
GGAGGAGGAGGATGCTGCAACCTATTACTGTCAGCACAGTAGGGAGCTT
CCGCTCACGTTCGGTGCTGGGACCAAGCTGGAGCTGAAA
>Protein
(SEQ ID NO: 163)
DIVLIQSPASLAVSLGQRATISCRASKSVSTSGYSYMHWFQQKPGQPPK
LLIYLASNLESGVPARFSGSGSGTDFTLNIHPVEEEDAATYYCQHSREL
PLTFGAGTKLELK

### Example 5

### In Vitro Relative Potency (IVRP) Assay for PE-PilA Fusion Protein LVL-735

The PE-PilA sandwich ELISA uses a mouse anti-PE monoclonal antibody (mAb ProtE/5) as capture antibody and a purified biotinylated mouse anti-PilA monoclonal antibody (mAb PEPilA/3) as detection antibody.

96 well ELISA plates were coated with ProtE/5 antibody for 1 h at 37° C. After washing, the plate was saturated for 30 minutes at 25° C. with BSA 1%. Reference standard (RS), internal control (IC) and test samples (i.e. LVL-735 fusion protein) were then diluted in assay diluent to a starting concentration of 0.3 μg/ml based on UPLC concentration and added to the first well of the ELISA plate, followed by a seven three-fold serial dilution (from line A to H).

After 30 minutes at 25° C., the plate was washed four times with NaCl 0.9% Tween 0.05% and the anti-PilA antibody (mAb PEPilA/3) was added and incubated for 30 minutes at 25° C. After re-washing, peroxidase-conjugated streptavidin was added and incubated for another 30 minutes at 25° C. The antigen-antibody complex was revealed by addition of o-phenylenediamine dihydrochloride (OPD) as peroxidase substrate. The enzymatic reaction was then stopped with chloridric acid (1 N) and the absorbance at 490 nm and 620 nm was measured. The absorbance vs. log (concentration) sigmoidal dose response curves were generated for RS, IC and each test sample. The curves were analysed using four parameter logistical fit model by SoftMax Pro software. Relative potency was determined via EC₅₀ calculation from global fitting test sample curve to RS curve. The internal control was used to validate each ELISA plate.

### Example 6

### Use of In Vitro Relative Potency Assay to Detect Thermally Stressed PE-PilA Fusion Protein LVL-735

In order to assess the ability of the assay (described in Example 5) to detect thermally stressed material, a PE-PilA drug substance lot was incubated at 60° C. for 8 hours. Samples were collected at T0, 1, 2, 3, 6 and 8 hours of incubation to evaluate the potential kinetic of antigen binding. The incubations were repeated 8 times.

Method: Microtiter 96-well plates (MAXISORP™, Nunc Thermo Scientific) were coated 1 hour at 37° C. with 100 μl per well of ProtE/5 purified mAb at 5.8 μg/ml diluted in carbonate buffer (pH 9.6). The plates were then washed four times with NaCl 0.9% Tween 0.05% and blocked for 30 min at 25° C. with 200 μl of saturation buffer [Phosphate buffer saline (PBS)+Bovine Serum Albumine (BSA) 1%]. After washing, reference, internal control and samples were added at 0.3 μg/ml in first well then diluted from line A to H according a 3-fold serial dilution in PBS Tween 20 0.05%. Reference and internal control were included in each test. The plates were then incubated for 30 min at 25° C. After washing, biotinylated PEPilA/3 mAb was added at 0.296 μg/ml (100 μl per well) for 30 min at 25° C. After re-washing, peroxidase-conjugated streptavidin was added and incubated for another 30 minutes at 25° C. Plates were re-washed as above and the solution of revelation [4 mg of OPDA and 5 μl of H2O2 in 10 ml of citrate 0.1 M pH 4.5 buffer] was added to each well (100 μl per well) for 15 min in darkness. The reaction was stopped by addition of 50 μl of HCl 1N and the optical density (OD) is read at 490 nm (620 nm for the reference filter). Relative potency (% versus the reference sample) for each sample is determined by full logistic curve parallelism method using SoftMax Pro software.

Results: As illustrated in Table 3 below a decrease in PE-PilA antigenicity (compared to an un-stressed reference standard) is observed for all incubations. The antigenicity decreased gradually to reach very low levels (between 3 and 7%) at 8 hours of incubation.

TABLE 3
PE-PilA antigenicity in thermally stressed material. Incubation
was performed at +60° C. for 8 hours.

| | PE-PilA antigenicity in % (compared to reference standard) | | | | | | | |
|---|---|---|---|---|---|---|---|---|
| Hours of | Sam- | Sam- | Sam- | Sam- | Sam- | Sam- | Sam- | Sam- |
| incubation | ple 1 | ple 2 | ple 3 | ple 4 | ple 5 | ple 6 | ple 7 | ple 8 |
| 0 | 113 | 107 | 100 | 88 | 101 | 115 | 107 | 80 |
| 1 | 91 | 90 | 105 | 72 | 111 | 84 | 67 | 65 |
| 2 | 68 | 67 | 77 | 67 | 75 | 50 | 48 | 43 |
| 3 | 50 | 46 | 55 | 41 | 60 | 35 | 33 | 24 |
| 6 | 10 | 12 | 14 | 12 | 14 | 9 | 13 | 7 |
| 8 | 5 | 4 | 6 | 5 | 7 | 4 | 5 | 3 |

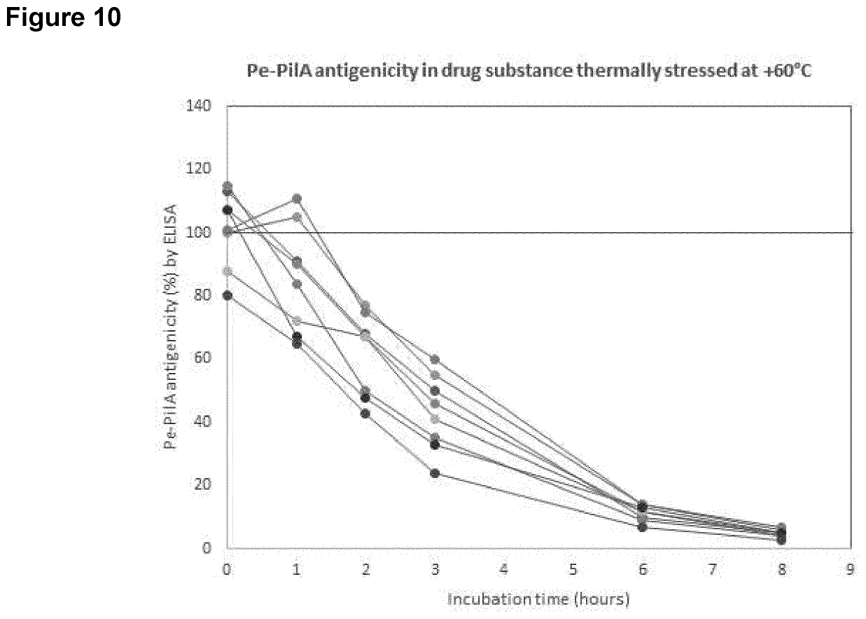

Graphically (see FIG. 10), a linear decrease in PE-PilA antigenicity is observed until 6 hours of incubation at +60° C. After 6 hours the slope of the kinetic decrease to reach the low levels of Pe-PilA antigenicity observed at 8 hours.

### Example 7

### Use of In Vitro Relative Potency Assay (IVRP) as a Stability-Indicating Method (SIM)

Aim: to further characterize the use of the IVRP assay (method described in Example 5) to evaluate stability-indicating properties of the PE-PilA fusion protein LVL-735 (SEQ ID NO: 122).

The LVL-735 fusion protein drug-substance (DS) used was a batch produced at final-scale (ENHPHPA009). The composition of the lot was identical to the final process: PE-PilA 1.25 mg/ml, 10 mM KH₂PO₄/K₂HPO₄, Poloxamer 188 0.2% (w/v), pH 6.5.

The LVL-735 drug substance was subjected to a first-screening which assessed the impact of the following stressors on antigenicity using the IVRP assay (as described in Example 5)
Heat stress (3 weeks at 37° C., 24-hours at either 50° C., 60° C. or 70° C.),
Light Exposure performed using the Accelerated Oxidative Test (AOT) where the drug substance is exposed to light within a chamber for 15 h, at 765 W/m2/320-380 nM),
Methionine oxidation (spike 0.06% H2O2 for 24 h at 25° C.),
pH (24 hours at pH 4 and pH 7 at 25° C.
Trypsin treatment (60-minute incubation at 37° with 1:100 ratio of trypsin to PE-PilA (i.e. 2.5 μg immobilized TPCK trypsin (ThermoFisher Cat #20230) to 250 μg PE-PilA drug substance)

The data is shown in table 4 below as unique values obtained relative to the reference standard (wherein the reference standard is the BSCPEPIL01 clinical lot as used in a proof-of-concept clinical trial (1193 μg/ml).

| Sample | Relative Potency (%) |
|---|---|
| ENHPGPA009 Control (Untreated) | 110 |
| ENHPGPA009 3 week 37° C. | 102 |
| ENHPGPA009 24-h 50° C. | 103 |
| ENHPGPA009 24-h 60° C. | 0 |
| ENHPGPA009 24-h 70° C. | 0 |
| ENHPGPA009 AOT | 94 |
| ENHPGPA009 H202 | 117 |
| ENHPGPA009 pH 4 | 97 |
| ENHPGPA009 pH 9 | 100 |
| ENHPGPA009 Trypsin | 18 |

Results: PE-PilA antigenic activity was not affected by the following treatments: 3 weeks at +37° C., 24 h at +50° C., AOT, H₂O₂, pH 4 and pH 9. No activity could be measured after 24 h of incubation at +60° C. or +70° C. After trypsin treatment, the antigenic activity was strongly reduced (18% residual activity).

### Example 8

### Further Assessment of the Antigenic Activity of Thermally Stressed PE-PilA Fusion Protein

The use of the IVRP assay (as described in Example 5) to evaluate the antigenicity of stressed material (i.e. samples of fusion protein LVL-735 (SEQ ID NO: 122)) was further evaluated.

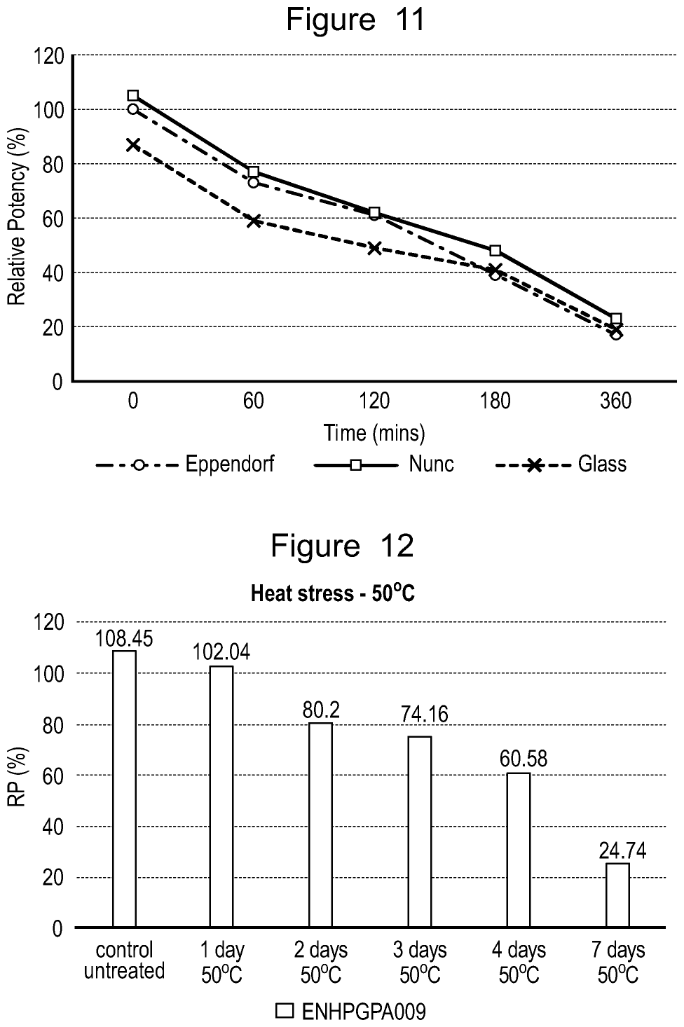

Incubation of PE-PilA fusion protein LVL-735 (batch ENHPGPA009) at +60° C. was performed in different containers (Eppendorf tubes, polypropylene NUNC tubes and glass vials), for durations between 1 and 6 hours, and the PE-PilA antigenic activity was measured as unique values by ELISA (see FIG. 11, n=1).

A progressive decrease of antigenic activity was observed in all types of containers. Slightly lower ELISA results were obtained for samples incubated at +60° C. in glass vials (at T0 and after 60 min and 120 min). Note that after 6 h of thermal stress at 60° C., there appeared to be precipitate in samples.

To further elucidate the utility of the IVRP assay for detecting decline in antigenicity due to heat stress, the PE-PilA fusion protein LVL-735 of SEQ ID NO: 122 (ENHPGPA009 lot i.e. batch of LVL-735 drug substance produced at final scale) was subjected to degradation at 50° C. for up to 7 days (see FIG. 12). Heat stress induced a loss of IVRP of 24.74% residual activity after 7-days at +50° C.

Sequence Listing

SEQ ID NO. 1 (Protein E) - Corresponding to SEQ ID NO. 4 of WO2012/139225A1)
MKKIILTLSL GLLTACSAQI QKAEQNDVKL APPTDVRSGY IRLVKNVNYY IDSESIWVDN
QEPQIVHFDA VVNLDKGLYV YPEPKRYARS VRQYKILNCA NYHLTQVRTD FYDEFWGQGL
RAAPKKQKKH TLSLTPDTTL YNAAQIICAN YGEAFSVDKK

| Strain Name | Protein E sequence |
|---|---|
| 3224A | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHTLSLTPDTTLYNAA |
| | QIICANYGEAFSVDKK (SEQ ID NO. 2) |
| RdKW20 | MKKIILTLSLGLLTACSAQIQKAEQNDVKLAPPTDVRSGYIRLVKNVNYYIDSESIWVDNQEPQIVHFDAVV |
| | NLDRGLYVYPEPKRYARSVRQYKILNCANYHLTQIRTDFYDEFWGQGLRAAPKKQKKHTLSLTPDTTLYNAA |
| | QIICANYGKAFSVDKK (SEQ ID NO. 3) |
| 86-028NP | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHTLSLTPDTTLYNAA |
| | QIICANYGEAFSVDKK (SEQ ID NO. 4) |
| R2846 | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHTLSLTPDTTLYNAA |
| | QIICANYGKAFSVDKK (SEQ ID NO. 5) |
| R2866 | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHTLSLTPDTTLYNAA |
| | QIICANYGKAFSVDKK (SEQ ID NO. 6) |
| 3655 | MKKIILTLSLGLLTACSAQIQKAEQNDMKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHTLSLTPDTTLYNAA |
| | QIICANYGKAFSVDKK (SEQ ID NO. 7) |
| PittAA | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHTLSLTPDTTLYNAA |
| | QIICANYGEAFSVDKK (SEQ ID NO. 8) |
| PittEE | MKKIILTLSLGLLTACSAQIQKAEQNDMKLAPPTDVRSGYIRLVKNVNYYIDSESI-VDNQEPQ |
| | IVHFDAVVNLDKGLYVYPEPKRYARSVRQYKILNCANYHLTQVRTDFYDEFWGQGLRAAPKKQKKHTLSLTP |
| | DTTLYNAAQIICANYGKAFSVDKK (SEQ ID NO. 9) |
| PittHH | MKKIILTLSLGLLTACSAQIQKAEQNDVKLAPPTDVRSGYIRLVKNVNYYIDSESIWVDNQEPQIVHFDTVV |
| | NLDKGLYVYPEPKRYARSVRQYKILNCANYHLTQVRTDFYDEFWGQGLRAAPKKQKKHTLSLTPDTTLYNAA |
| | QIICANYGKAFSVDKK (SEQ ID NO. 10) |
| PittII | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 11) |
| R3021 | MKKIILTLSLGLLTACSAQTQKAEQNDVKLIPPTDVQSGYVALVKNVNYYIDSESIWVDNQEPQIVHFDAVV |
| | NLDKGLYVYPEPKRYARSVRQYKILNCANYHLTQVRIDFYDEFWGQGLRAAPKKQKKHILSLTPDTTLYNAA |
| | QIICANYGKAFSVDKNKKICT-LISLNFIQLLGCREYSIFLQLLLFYC |
| | WHF (SEQ ID NO. 12) |
| 22.4-21 | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKKIKKICTLISLNFIQLLGCREYSIFLQLLLFYCWHF (SEQ ID NO. 13) |
| 3219C | MKKIILTLSLGLLTACSAQIQKAEQNDMKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 14) |
| 3185 | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 15) |
| 3241A | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGEAFSVDKK (SEQ ID NO. 16) |
| 038144S1 | MKKIILTLSLGLLTACSAQTQKVEQNDVKLTAPTDVRSGFVRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFLVDKK (SEQ ID NO. 17) |
| 810956 | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGEAFSVDKK (SEQ ID NO. 18) |
| 821246 | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQIRTDFYDEFWGQGLRAAPKKQKKHILSLTPDTTLYNAA |
| | QIICANYGKAFSVDKK (SEQ ID NO. 19) |
| 840645 | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGEAFSVDKK (SEQ ID NO. 20) |
| 902550Z19 | MKKIILTLSLGLLTACSAQTQKVEQNDVKLIPPTDVRSGYVALVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 21) |
| A840177 | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGEAFSVDKK (SEQ ID NO. 22) |
| A860514 | MKKIILTLSLGLLTACSAQTQKVEQNDVKLTAPTDVRSGYVALVKNANYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 23) |
| A950014 | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRIDFYDEFWGQGLRAAPKKQKKHILSLTPDTTLYNAA |
| | QIICANYGKAFSVDKK (SEQ ID NO. 24) |
| 306543X4 | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 25) |
| A930105 | MKKIILTLSLGLLTACSAQIQKAEQNDVKLAPPTDVRSGYIRLVKNVNYYIDSESIWVDNQEPQIVHFDTVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 26) |
| 901905U | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 27) |
| A920030 | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGEAFSVDKK (SEQ ID NO. 28) |
| 3221B | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 29) |
| 27W116791N | MKKIILTLSLGLLTACSAQTQKVEQNDVKLIPPTDVRSGYVALVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 30) |
| N218 | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 31) |
| N163 | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGEAFSVDKK (SEQ ID NO. 32) |
| N162 | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGEAFSVDKK (SEQ ID NO. 33) |
| N107 | MKKIILTLSLGLLTACSAQTQKAEQNDVKLAPPTDVRSGYIRLVKNVNYYIDSESIWVDNQEPQIVHFDAVV |
| | NLDKGLYVYPEPKRYARSVRQYKILNCANYHLTQIRTDFYDEFWGQGLRAAPKKQKKHILSLTPDTTLYNAA |
| | QIICANYGKAFSVDKK (SEQ ID NO. 34) |
| N91 | MKKIILTLSLGLLTACSAQTQKVEQNDVKLTAPADVRSGYVALVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 35) |
| D211PG | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVR-YKILNCANYHLTQVRTDFYDEFWGQGLRAAPKKQK |
| | KHILSLTPDTTLYNAAQIICANYGEAFSVDKK (SEQ ID NO. 36) |
| D211PD | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVR-YKILNCANYHLTQVRTDFYDEFWGQGLRAAPKKQK |
| | KHILSLTPDTTLYNAAQIICANYGEAFSVDKK (SEQ ID NO. 37) |
| D201PG | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 38) |
| D201PD | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 39) |
| D198PG | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 40) |
| D198PD | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 41) |
| D195PD | MKKIILTLSLGLLTACSAQIQKAEQNDVKLAPPTDVRSGYIRLVKNVNYYIDSESIWVDNQEPQIVHFDTVV |
| | NLDKGLYVYPEPKRYARSVRQYKILNCANYHLTQVRTDFYDEFWGQSLRAAPKKQKKHILSLTPDTTLYNAA |
| | QIICANYGKAFSVDKK (SEQ ID NO. 42) |
| D189PG | MKKIILTLSLGLLTACSAQTQKVEQNDVKLIPPTDVRSGYVALVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTIVYNAA |
| | QIICANYGKAFSVDKK (SEQ ID NO. 43) |
| D189PD | MKKIILTLSLGLLTACSAQTQKVEQNDVKLIPPTDVRSGYVALVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTIVYNAA |
| | QIICANYGKAFSVDKK (SEQ ID NO. 44) |
| D129CG | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGEAFSVDKK (SEQ ID NO. 45) |
| D124PG | MKKIILTLSLGLLTACSAQIQKAEQNDVKLAPPTDVRSGYIRLVKNVNYYIDSESIWVDNQEPQIVHFDTVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 46) |
| D124PD | MKKIILTLSLGLLTACSAQIQKAEQNDVKLAPPTDVRSGYIRLVKNVNYYIDSESIWVDNQEPQIVHFDTVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 47) |
| D58PG | MKKIILTLSLGLLTACSAQTQKAEQNDVKLIPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 48) |
| D33OD | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 49) |
| BS433 | MKKIILTLSLGLLTACSAQIQKAEQNDVKLAPPTDVRSGYIRLVKNVNYYIDSESIWVDNQEPQIVHFDTVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 50) |
| BS432 | MKKIILTLSLGLLTACSAQTQKAEQNDVKLAPPTDVRSGYIRLVKNVNYYIDSESIWVDNQEPQIVHFDAVV |
| | NLDKGLYVYPEPKRYARSVRQYKILNCANYHLTQIRTDFYDEFWGQGLRAAPKKQKKHILSLTPDTTLYNAA |
| | QIICANYGKAFSVDKK (SEQ ID NO. 51) |
| 1714 | MKKIILTLSLGLLTACSAQIQKAKQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGEAFSVDKK (SEQ ID NO. 52) |
| 1128 | MKKIILTLSLGLLTACSAQIQKAEQNDVKLAPPTDVRSGYIRLVKNVNYYIDSESIWVDNQEPQIVHFDAVV |
| | NLDKGLYVYPEPKRYARSVRQYKILNCANYHLTQVRTDFYDEFWGQGLRAAPKKQKKHILSLTPDTTLYNAA |
| | QIICANYGKAFSVDKK (SEQ ID NO. 53) |
| BS430 | MKKIILTLSLGLLTACSAQIQKAEQNDMKLAPPTDVRSGYIRLVKNVNYYIDSESI-VDNQEPQ |
| | IVHFDAVVNLDKGLYVYPEPKRYARSVRQYKILNCANYHLTQVRTDFYDEFWGQGLRAAPKKQKKHILSLIP |
| | DTTLYNAAQIICANYGKAFSVDKK (SEQ ID NO. 54) |

SEQ ID NO. 55: Amino acids 20-160 of Protein E
I QKAEQNDVKL APPTDVRSGY IRLVKNVNYY IDSESIWVDN QEPQIVHFDA VVNLDKGLYV YPEPKRYARS
VRQYKILNCA NYHLTQVRTD FYDEFWGQGL RAAPKKQKKH TLSLTPDTTL YNAAQIICAN YGEAFSVDKK
SEQ ID NO: 56 (corresponding to SEQ ID NO. 58 from WO2012/139225A1) - PlLA
MKLTTQQTLK KGFTLIELMI VIAIIAILAT IAIPSYQNYT KKAAVSELLQ ASAPYKADVE
LCVYSTNETT NCTGGKNGIA ADITTAKGYV KSVTTSNGAI TVKGDGTLAN MEYILQATGN
AATGVTWTTT CKGTDASLFP ANFCGSVTQ

| Strain Name | PilA sequence |
|---|---|
| 86-028NP | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 56) |
| NTHi3219C | MKLTTQQTLKKGFTLIELMIVIAIIAILATIAIPSYQNYTKKAAVSELLQASAPYKADVELCVYSTNETTKC |
| | TGGKNGIAADITTAKGYVKSVTTSNGAITVAGNGTLDGMSYTLTAEGDSAKGVTWKTTCKGTDASLFPANFC |
| | GSVTQ (SEQ ID NO. 57) |
| NTHi3224A | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 58) |
| NTHi12 | MKLTTQQTLKKGFTLIELMIVIAIIAILATIAIPSYKNYTKKAAVSELLQASAPYKADVELCVYSTGKPSSC |
| | SGGSNGIAADITTAKGYVASVITQSGGITVKGDGTLANMEYILQAAGNAAAGVTWTTTCKGTDASLFPANFC |
| | GSVTQ (SEQ ID NO. 59) |
| NTHi44 | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 60) |
| NTHi67 | MKLTTQQTLKKGFTLIELMIVIAIIAILATIAIPSYQNYTKKAAVSELLQASAPYKSDVELCVYSTGKPSTC |
| | SGGSNGIAADITTVKGYVKSVTTSNGAITVAGNGTLDGMSYTLTAEGDSAKGVTWTTTCKGTDASLFPANFC |
| | GSVTQ (SEQ ID NO. 61) |
| 1054MEE | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 62) |
| 1729MEE | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 63) |
| 1728MEE | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 64) |
| 1885MEE | MKLTTQQTLKKGFTLIELMIVIAIIAILATIAIPSYKNYTKKAAVSELLQASAPYKADVELCVYSTNEITNC |
| | MGGKNGIAADITTAKGYVASVKTQSGGITVKGDGTLANMEYILQATGNAAAGVTWTTTCKGTDASLFPANFC |
| | GSITQ (SEQ ID NO. 65) |
| 1060MEE | MKLTTQQTLKKGFTLIELMIVIAIIAILATIAIPSYQNYTKKASVSELLQASAPYKADVELCVYSTNETTNC |
| | TGGKNGIAADITTAKGYVASVKTQSGGITVKGNGTLANMEYILQAKGNATAGVTWTTTCKGTDASLFPANFC |
| | RSVTK (SEQ ID NO. 66) |
| RdKW20 | MKLTTLQTLKKGFTLIELMIVIAIIAILATIAIPSYQNYTKKAAVSELLQASAPYKADVELCVYSTNETTSC |
| | TGGKNGIAADIKTAKGYVASVITQSGGITVKGNGTLANMEYILQAKGNAAAGVTWTTTCKGTDASLFPANFC |
| | GSVTK (SEQ ID NO. 67) |
| 214NP | MKLTTQQTLKKGFTLIELMIVIAIIAILATIAIPSYQNYTKKAAVSELLQASAPYKADVELCVYSTGKPSSC |
| | SGGSNGIAADITTAKGYVKSVTTSNGAITVKGDGTLANMEYILQASGNAATGVTWTTTCKGTDASLFPANFC |
| | GSVTQ (SEQ ID NO. 68) |
| 1236MEE | MKLTTLQTLKKGFTLIELMIVIAIIAILATIAIPSYQNYTKKAAVSELLQASAPYKADVELCVYSTNETTSC |
| | TGGKNGIAADIKTAKGYVASVITQSGGITVKGNGTLANMEYILQAKGNAAAGVTWTTTCKGTDASLFPANFC |
| | GSVTK (SEQ ID NO. 69) |
| 1714MEE | MKLTTLQTLKKGFTLIELMIVIAIIAILATIAIPSYQNYTKKAAVSELLQASAPYKADVELCVYSTGKPSTC |
| | SGGSNGIAADITTAKGYVASVKTQSGGITVKGNGTLANMEYILQATGNAATGVTWTTTCKGTDASLFPANFC |
| | GSVTQ (SEQ ID NO. 70) |
| 1128MEE | MKLTTQQTLKKGFTLIELMIVIAIIAILATIAIPSYQNYTKKASVSELLQASAPYKSDVELCVYSTGKPSTC |
| | SGGSNGIAADITTAKGYVASVKTQSGGITVKGNGTLANMEYILQAKGNATAGVTWTTTCKGTDASLFPANFC |
| | RSVTK (SEQ ID NO. 71) |
| R2846 | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 72) |
| R2866 | MKLTTQQTLKKGFTLIELMIVIAIIAILATIAIPSYQNYTKKAAVSELLQASAPYKADVELCVYSTNETTNC |
| | TGGKNGIAADITTAKGYVASVKTQSGGITVKGDGTLANMEYILQATGNAATGVTWTTTCKGTEASLFPANFC |
| | GSVTQ (SEQ ID NO. 73) |
| 3655 | MKLTTQQTLKKGFTLIELMIVIAIIAILATIAIPSYQNYTKKASVSELLQASAPYKADVELCVYSTNETTNC |
| | TGGKNGIAADITTAKGYVASVKTQSGGITVKGNGTLANMEYILQAKGNATAGVTWTTTCKGTDASLFPANFC |
| | RSVTK (SEQ ID NO. 74) |
| PittAA | MKLTTLQTLKKGFTLIELMIVIAIIAILATIAIPSYQNYTKKAAVSELLQASAPYKADVELCVYSTGKPSTC |
| | SGGSNGIAADITTAKGYVASVKTQSGGITVKGNGTLANMEYILQATGNAATGVTWTTTCKGTDASLFPANFC |
| | GSVTQ (SEQ ID NO. 75) |
| PittGG | MKLTTQQTLKKGFTLIELMIVIAIIAILATIAIPSYQNYTKKAAVSELLQASAPYKADVELCVYSTGKPSTC |
| | SGGSNGIAADITTAKGYVASVKTQSGGITVKGNGTLANMEYILQAKGNATAGVTWTTTCKGTDASLFPANFC |
| | RSVTK (SEQ ID NO. 76) |
| PittII | MKLTTQQTLKKGFTLIELMIVIAIIAILATIAIPSYQNYTKKAAVSELLQASAPYKADVELCVYSTNETTNC |
| | TGGKNGIAADITTAKGYVASVKTQSGGITVKGDGTLANMEYILQATGNAATGVTWTTTCKGTEASLFPANFC |
| | GSVTQ (SEQ ID NO. 77) |
| R3021 | MKLTTQQTLKKGFTLIELMIVIAIIAILATIAIPSYQNYTKKAAVSELLQASAPYKADVELCVYSTNETTNC |
| | TGGKNGIAADITTAKGYVASVKTQSGGITVKGDGTLANMEYILQATGNAATGVTWTTTCKGTEASLFPANFC |
| | GSVTQ (SEQ ID NO. 78) |
| 22.4-21 | MKLTTQQTLKKGFTLIELMIVIAIIAILATIAIPSYQNYTKKAAVSELLQASAPYKSDVELCVYSTGKPSTC |
| | SGGSNGIAADITTAKGYVKSVTTSNGAITVAGNGTLDGMSYTLTAEGDSAKGVTWKTTCKGTDASLFPANFC |
| | GSVTK (SEQ ID NO. 79) |
| 3185A | MKLTTQQTLKKGFTLIELMIVIAIIAILATIAIPSYQNYTKKAAVSELLQASAPYKADVELCVYSTNEATKC |
| | TGGKNGIAADITTAKGYVKSVTTSNGAITVKGDGTLANMEYILQASGNAATGVTWTTTCKGTDASLFPANFC |
| | GSVTQ (SEQ ID NO. 80) |
| 3221B | MKLTTQQTLKKGFTLIELMIVIAIIAILATIAIPSYQNYTKKAAVSELLQASAPYKADVELCVYSTNEATKC |
| | TGGKNGIAADITTAKGYVKSVTTSNGAITVKGDGTLANMEYILQASGNAATGVTWTTTCKGTDASLFPANFC |
| | GSVTQ (SEQ ID NO. 81) |
| 3241A | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 82) |
| 038144S1 | MKLTTQQTLKKGFTLIELMIVIAIIAILATIAIPSYQNYTKKAAISELLQASAPYKSDVELCVYSTGKPSTC |
| | SGGSNGIAADITTAKGYVASVKTQSGGITVKGNGTLANMEYILQAKGNATAGVTWTTTCKGTDASLFPANFC |
| | RSVTK (SEQ ID NO. 83) |
| 821246 | MKLTTQQTLKKGFTLIELMIVIAIIAILATIAIPSYQNYTKKAAVSELLQASAPYKADVELCVYSTNETTNC |
| | TGGKNGIAADITTAKGYVASVKTQSGGITVKGDGTLANMEYILQATGNAATGVTWTTTCKGTEASLFPANFC |
| | GSVTQ (SEQ ID NO. 84) |
| 840645 | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 85) |
| 902550Z19 | MKLTTQQTLKKGFTLIELMIVIAIIAILATIAIPSYQNYTKKAAVSELLQASAPYKSDVELCVYSTGKPSTC |
| | SGGSNGIAADITTVKGYVKSVTTSNGAITVAGNGTLDGMSYTLTAEGDSAKGVTWTTTCKGTDASLFPANFC |
| | GSVTQ (SEQ ID NO. 86) |
| A840177 | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 87) |
| A920030 | MKLTTQQTLKKGFTLIELMIVIAIIAILATIAIPSYQNYTKKAAVSELLQASAPYKADVELCVYSTNETTNC |
| | TGGKNGIAADITTAKGYVASVKTQSGGITVKGNGTLANMEYILQATGNAATGVTWTTTCKGTDASLFPANFC |
| | GSVTQ (SEQ ID NO. 88) |
| A950014 | MKLTTQQTLKKGFTLIELMIVIAIIAILATIAIPSYQNYTKKAAVSELLQASAPYKADVELCVYSTGKPSTC |
| | SGGSNGIAADITTAKGYVKSVTTSNGAITVAGNGTLDRMSYTLTAEGDSAKGVTWTTTCKGTDASLEPANFC |
| | GSVTQ (SEQ ID NO. 89) |
| 901905U | MKLTTQQTLKKGFTLIELMIVIAIIAILATIAIPSYQNYTKKAAVSELLQASAPYKADVELCVYSTGKPSSC |
| | SGGSNGIAADITTAKGYVKSVTTSNGAITVKGDGTLANMEYILQASGNAATGVTWTTTCKGTDASLFPANFC |
| | GSVTQ (SEQ ID NO. 90) |
| A920029 | MKLTTQTTLKKGFTLIELMIVIAIIAILATIAIPSYQNYTKKAAVSELLQASAPYKSDVELCVYSTNETTNC |
| | TGGKNGIAADITTAKGYVASVITQSGGITVKGNGTLTNMEYILQATGNAATGVTWTTTCKGTDASLFPANFC |
| | GSITQ (SEQ ID NO. 91) |
| A930105 | MKLTTLQTLKKGFTLIELMIVIAIIAILATIAIPSYQNYTKKAAVSELLQASAPYKADVELCVYSTGKPSTC |
| | SGGNNGIAADIKTAKGYVASVKTQSGGITVKGDGTLANMEYILQATGNAATGVTWTTTCKGTDASLFPANFC |
| | GSVTQ (SEQ ID NO. 92) |
| 306543X4 | MKLTTQQTLKKGFTLIELMIVIAIIAILATIAIPSYQNYTKKAAVSELLQASAPYKADVELCVYSTGKPSSC |
| | SGGSNGIAADITTAKGYVKSVTTSNGAITVKGDGTLANMEYILQASGNAATGVTWTTTCKGTDASLFPANFC |
| | GSVTQ (SEQ ID NO. 93) |
| N218 | MKLTTQQTLKKGFTLIELMIVIAIIAILATIAIPSYQNYTKKAAVSELLQASAPYKADVELCVYSTNEATKC |
| | TGGKNGIAADITTAKGYVKSVTTSNGAITVKGDGTLANMEYILQASGNAATGVTWTTTCKGTDTSLFPANFC |
| | GSVTQ (SEQ ID NO. 94) |
| N163 | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 95) |
| N162 | MKLTTQQTLKKGFTLIELMIVIAIIAILATIAIPSYQNYTKKAAVSELLQASAPYKADVELCVYSTNETTNC |
| | TGGKNGIAADITTAKGYVASVKTQSGGITVKGNGTLANMEYILQATGNAATGVTWTTTCKGTDASLFPANFC |
| | GSVTQ (SEQ ID NO. 96) |
| N120 | MKLTTQQTLKKGFTLIELMIVIAIIAILATIAIPSYQNYTKKAAVSELLQASAPYKADVELCVYSTGKPSTC |
| | SGGSNGIAADITTAKGYVASVKTQSGGITVKGNGTLANMEYILQAKGNATAGVTWTTTCKGTDASLFPANFC |
| | RSVTK (SEQ ID NO. 97) |
| N107 | MKLTTQQTLKKGFTLIELMIVIAIIAILATIAIPSYQNYTKKAAVSELLQASAPYKADVELCVYSTGKPSTC |
| | SGGSNGIAADITTAKGYVASVKTQSGGITVKGNGTLANMEYILQAKGNATAGVTWTTTCKGTDASLFPANFC |
| | RSVTK (SEQ ID NO. 98) |
| N92 | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 99) |
| N91 | MKLTTQQTLKKGFTLIELMIVIAIIAILATIAIPSYQNYTKKAAVSELLQASAPYKADVELCVYSTGKPSTC |
| | SGGSNGIAADITTAKGYVASVKTQSGGITVKGNGTLANMEYILQAKGNATAGVTWTTTCKGTDASLFPANFC |
| | RSVTK (SEQ ID NO. 100) |
| D219PG | MKLTTQQTLKKGFTLIELMIVIAIIAILATIAIPSYQNYTKKAAVSELLQASAPYKADVELCVYSTNEATKC |
| | TGGKNGIAADITTAKGYVKSVTTSNGAITVKGDGTLANMEYILQASGNAATGVTWTTTCKGTDASLFPANFC |
| | GSVTQ (SEQ ID NO. 101) |
| D211PG | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 102) |
| D211PD | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 103) |
| D204CD | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILXATGNAATGVTWTTTCKGTDASLFPANFC |
| | GSVTQ (SEQ ID NO. 104) |
| D198PG | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 105) |
| D198PD | MKLTTQQTLKKGFTLIELMIVIAIIAILATIAIPSYQNYTKKAAVSELLQASAPYKADVELCVYSTNETTNC |
| | TGGKNGIAADITTAKGYVKSVTTSNGAITVKGDGTLANMEYILQATGNAATGVTWTTTCKGTDASLFPANFC |
| | GSVTQ (SEQ ID NO. 106) |
| D195PD | MKLTTLQTLKKGFTLIELMIVIAIIAILATIAIPSYQNYTKKAAVSELLQASAPYKADVELCVYSTGKPSTC |
| | SGGNNGIAADIKTAKGYVASVKTQSGGITVKGDGTLANMEYILQATGNAATGVTWTTTCKGTDASLFPANFC |
| | GSVTQ (SEQ ID NO. 107) |
| D195CD | MKLTTLQTLKKGFTLIELMIVIAIIAILATIAIPSYQNYTKKAAVSELLQASAPYKADVELCVYSTGKPSTC |
| | SGGNNGIAADIKTAKGYVASVKTQSGGITVKGDGTLANMEYILQATGNAATGVTWTTTCKGTDASLFPANFC |
| | GSVTQ (SEQ ID NO. 108) |
| D189PG | MKLTTQQTLKKGFTLIELMIVIAIIAILATIAIPSYQNYTKKAAVSELLQASAPYKADVELCVYSTNETTSC |
| | TGGKNGIAADITTAKGYVKSVTTSNGAITVAGNGTLDGMSYTLTAEGDSAKGVTWKTTCKGTDASLFPANFC |
| | GSVTQ (SEQ ID NO. 109) |
| D189PD | MKLTTQQTLKKGFTLIELMIVIAIIAILATIAIPSYQNYTKKAAVSELLQASAPYKADVELCVYSTNETTSC |
| | TGGKNGIAADITTAKGYVKSVTTSNGAITVAGNGTLDGMSYTLTAEGDSAKGVTWKTTCKGTDASLFPANFC |
| | GSVTQ (SEQ ID NO. 110) |
| D124PG | MKLTTLQTLKKGFTLIELMIVIAIIAILATIAIPSYQNYTKKAAVSELLQASAPYKADVELCVYSTGKPSTC |
| | SGGNNGIAADIKTAKGYVASVKTQSGGITVKGDGTLANMEYILQATGNAATGVTWTTTCKGTDASLFPANFC |
| | GSVTQ (SEQ ID NO. 111) |
| D124PD | MKLTTLQTLKKGFTLIELMIVIAIIAILATIAIPSYQNYTKKAAVSELLQASAPYKADVELCVYSTGKPSTC |
| | SGGNNGIAADIKTAKGYVASVKTQSGGITVKGDGTLANMEYILQATGNAATGVTWTTTCKGTDASLFPANFC |
| | GSVTQ (SEQ ID NO. 112) |
| D124CG | MKLTTLQTLKKGFTLIELMIVIAIIAILATIAIPSYQNYTKKAAVSELLQASAPYKADVELCVYSTGKPSTC |
| | SGGNNGIAADIKTAKGYVASVKTQSGGITVKGDGTLANMEYILQATGNAATGVTWTTTCKGTDASLFPANFC |
| | GSVTQ (SEQ ID NO. 113) |
| D58PG | MKLTTQQTLKKGFTLIELMIVIAIIAILATIAIPSYQNYTKKAAVSELLQASAPYKADVELCVYSTNETTNC |
| | TGGKNGIAADITTAKGYVASVKTQSGGITVKGDGTLANMEYILQATGNAATGVTWTTTCKGTEASLFPANFC |
| | GSVTQ (SEQ ID NO. 114) |
| BS433 | MKLTTLQTLKKGFTLIELMIVIAIIAILATIAIPSYQNYTKKAAVSELLQASAPYKADVELCVYSTGKPSTC |
| | SGGNNGIAADIKTAKGYVASVKTQSGGITVKGDGTLANMEYILQATGNAATGVTWTTTCKGTDASLFPANFC |
| | GSVTQ (SEQ ID NO. 115) |
| BS432 | MKLTTQQTLKKGFTLIELMIVIAIIAILATIAIPSYQNYTKKAAVSELLQASAPYKADVELCVYSTGKPSTC |
| | SGGSNGIAADITTAKGYVASVKTQSGGITVKGNGTLANMEYILQAKGNATAGVTWTTTCKGTDASLFPANFC |
| | RSVTK (SEQ ID NO. 116) |
| BS430 | MKLTTQQTLKKGFTLIELMIVIAIIAILATIAIPSYQNYTKKAAVSELLQASAPYKADVELCVYSTNEATKC |
| | TGGKNGIAADITTAKGYVKSVTTSNGAITVKGDGTLANMEYILQASGNAATGVTWTTTCKGTDASLFPANFC |
| | GSVTQ (SEQ ID NO. 117) |
| 1714 | MKLTTLQTLKKGFTLIELMIVIAIIAILATIAIPSYQNYTKKAAVSELLQASAPYKADVELCVYSTGKPSTC |
| | SGGSNGIAADITTAKGYVASVKTQSGGITVKGNGTLANMEYILQATGNAATGVTWTTTCKGTDASLFPANFC |
| | GSVTQ (SEQ ID NO. 118) |
| 1128 | MKLTTQQTLKKGFTLIELMIVIATIAILATIAIPSYQNYTKKASVSELLQASAPYKSDVELCVYSTGKPSTC |
| | SGGSNGIAADITTAKGYVASVKTQSGGITVKGNGTLANMEYILQAKGNATAGVTWTTTCKGTDASLEPANFC |
| | RSVTK (SEQ ID NO. 119) |

SEQ ID NO. 120 (corresponding to SEQ ID NO: 127 of WO2012/139225A1):
Amino acids 40-149 of PilA from H. influenzae strain 86-028NP
T KKAAVSELLQ ASAPYKADVE LCVYSTNETT NCTGGKNGIA ADITTAKGYV KSVTTSNGAI TVKGDGTLAN
MEYILQATGN AATGVTWTTT CKGTDASLFP ANFCGSVTQ.
SEQ ID NO. 121: LVL735 (protein): (pelB sp)(ProtE aa 20-160)(GG)(PilA aa40-149):
MKYLLPTAAA GLLLLAAQPA MAIQKAEQND VKLAPPTDVR SGYIRLVKNV NYYIDSESIW VDNQEPQIVH
FDAVVNLDKG LYVYPEPKRY ARSVRQYKIL NCANYHLTQV RTDFYDEFWG QGLRAAPKKQ KKHTLSLTPD
TTLYNAAQII CANYGEAFSV DKKGGTKKAA VSELLQASAP YKADVELCVY STNETTNCTG GKNGIAADIT
TAKGYVKSVT TSNGAITVKG DGTLANMEYI LQATGNAATG VTWTTTCKGT DASLFPANFC GSVTQ
SEQ ID NO. 122: PE-PilA fusion protein without signal peptide
IQKAEQND VKLAPPTDVR SGYIRLVKNV NYYIDSESIW VDNQEPQIVH FDAVVNLDKG LYVYPEPKRY
ARSVRQYKIL NCANYHLTQV RTDFYDEFWG QGLRAAPKKQ KKHTLSLTPD TTLYNAAQII CANYGEAFSV
DKKGGTKKAA VSELLQASAP YKADVELCVY STNETTNCTG GKNGIAADIT TAKGYVKSVT TSNGAITVKG
DGTLANMEYI LQATGNAATG VTWTTTCKGT DASLFPANFC GSVTQ
SEQ ID NO: 123 - ProtE/5 Variable Heavy (DNA)
CAGATCCAGTTGGTGCAGTCTGGACCTGAGCTGAAGAAGCCTGGAGAGACAGTCAAGATCTCCTGCAAGGCTTCTGGGTAT
ACCTTCACAAACTATGGAATGAACTGGGTGAAGCAGGCTCCAGGAAAGGGTTTAAAGTGGATGGGCTGGATAAACACCTAC
ACTGGAGAGCCAACATATGCTGATGACTTCAAGGGACGGTTTGCCTTCTCTTTGGAAACCTCTGCCAGCACTGCCTATTTG
CAGATCAACAACCTCAAAAATGAGGACACGGCTACATATTTCTGTGCAAGGGGAGGGTACCCCTCCTCGCGGGCGCCCCCT
TACTGGGGCCAAGGGACTCTGGTCACTGTCTCTGCAG
SEQ ID NO: 124 - ProtE/5 Variable Heavy (Protein)
QIQLVQSGPELKKPGETVKISCKASGYTFTNYGMNWVKQAPGKGLKWMGWINTYTGEPTYADDFKGRFAFSLETSASTAYL
QINNLKNEDTATYFCARGGYPSSRAPPYWGQGTLVTVSA
SEQ ID NO: 125 - Prot/E Variable Heavy (CDR-H1)
NYGMN
SEQ ID NO: 126 Prot/E Variable Heavy (CDR-H2)
WINTYTGEPTYADDFKG
SEQ ID NO: 127 Prot/E Variable Heavy (CDR-H3)
GGYPSSRAPPY
SEQ ID NO: 128 ProtE/5 Variable Light (DNA)
GACATTGTGATGACCCAGTCTCACAAATTCATGTCCACATCAGTAGGAGACAGGGTCAGCATCACCTGCAAGGCCAGTCAG
GATGTGGGTACTGCTGTAGCCTGGTATCAACAGAAACCAGGGCAATCTCCTAAACTACTGATTTACTGGGCATCCACCCGG
CACACTGGAGTCCCTGATCGCTTCACAGGCAGTGGATCTGGGACAGATTTCACTCTCACCATTAGCAATGTGCAGTCTGAA
GACTTGGCAGATTATTTCTGTCAGCAATATAGCAGCTATCCATTCACGTTCGGCTCGGGGACAAAGTTGGAAATAAAA
SEQ ID NO: 129 ProtE/5 Variable Light (Protein)
DIVMTQSHKFMSTSVGDRVSITCKASQDVGTAVAWYQQKPGQSPKLLIYWASTRHTGVPDRFTGSGSGTDFTLTISNVQSE
DLADYFCQQYSSYPFTFGSGTKLEIK
SEQ ID NO: 130 Prot/E Variable Light (CDR-L1)
KASQDVGTAVA
SEQ ID NO: 131 Prot/E Variable Light (CDR-L2)
WASTRHT
SEQ ID NO: 132 Prot/E Variable Light (CDR-L3)
QQYSSYPFT
SEQ ID NO: 133 - Protein E epitope region (I41 to Y49 of SEQ ID NO: 1)
IRLVKNVNY
SEQ ID NO: 134 - Protein E epitope region (Y141 to A154 of SEQ ID NO: 1)
YNAAQIICANYGEA
SEQ ID NO: 135 - PilA epitope region (C62 to A81 of SEQ ID NO: 56)
CVYSTNETTNCTGGKNGIAA
SEQ ID NO: 136 - Protein E Vitronectin Binding Domain (Corresponding to amino
acids 84-108 of SEQ ID NO: 1)
PKRYARSVRQYKILNCANYHLTQVR
SEQ ID NO: 137 - Protein E Laminin Binding Domain (Corresponding to amino acids
41-68 of SEQ ID NO: 1)
IRLVKNVNYYIDSESIWVDNQEPQIVHF
SEQ ID NO: 138 - Protein D
Met Lys Leu Lys Thr Leu Ala Leu Ser Leu Leu Ala Ala Gly Val Leu Ala Gly Cys Ser
Ser His Ser Ser Asn Met Ala Asn Thr Gln Met Lys Ser Asp Lys Ile Ile Ile Ala His
Arg Gly Ala Ser Gly Tyr Leu Pro Glu His Thr Leu Glu Ser Lys Ala Leu Ala Phe Ala
Gln Gln Ala Asp Tyr Leu Glu Gln Asp Leu Ala Met Thr Lys Asp Gly Arg Leu Val Val
Ile His Asp His Phe Leu Asp Gly Leu Thr Asp Val Ala Lys Lys Phe Pro His Arg His
Arg Lys Asp Gly Arg Tyr Tyr Val Ile Asp Phe Thr Leu Lys Glu Ile Gln Ser Leu Glu
Met Thr Glu Asn Phe Glu Thr Lys Asp Gly Lys Gln Ala Gln Val Tyr Pro Asn Arg Phe
Pro Leu Trp Lys Ser His Phe Arg Ile His Thr Phe Glu Asp Glu Ile Glu Phe Ile Gln
Gly Leu Glu Lys Ser Thr Gly Lys Lys Val Gly Ile Tyr Pro Glu Ile Lys Ala Pro Trp
Phe His His Gln Asn Gly Lys Asp Ile Ala Ala Glu Thr Leu Lys Val Leu Lys Lys Tyr
Gly Tyr Asp Lys Lys Thr Asp Met Val Tyr Leu Gln Thr Phe Asp Phe Asn Glu Leu Lys
Arg Ile Lys Thr Glu Leu Leu Pro Gln Met Gly Met Asp Leu Lys Leu Val Gln Leu Ile
Ala Tyr Thr Asp Trp Lys Glu Thr Gln Glu Lys Asp Pro Lys Gly Tyr Trp Val Asn Tyr
Asn Tyr Asp Trp Met Phe Lys Pro Gly Ala Met Ala Glu Val Val Lys Tyr Ala Asp Gly
Val Gly Pro Gly Trp Tyr Met Leu Val Asn Lys Glu Glu Ser Lys Pro Asp Asn Ile Val
Tyr Thr Pro Leu Val Lys Glu Leu Ala Gln Tyr Asn Val Glu Val His Pro Tyr Thr Val
Arg Lys Asp Ala Leu Pro Glu Phe Phe Thr Asp Val Asn Gln Met Tyr Asp Ala Leu Leu
Asn Lys Ser Gly Ala Thr Gly Val Phe Thr Asp Phe Pro Asp Thr Gly Val Glu Phe Leu
SEQ ID NO: 139 - Protein D fragment with MDP tripeptide from NS1
Met Asp Pro Ser Ser His Ser Ser Asn Met Ala Asn Thr Gln Met Lys Ser Asp Lys Ile
Ile Ile Ala His Arg Gly Ala Ser Gly Tyr Leu Pro Glu His Thr Leu Glu Ser Lys Ala
Leu Ala Phe Ala Gln Gln Ala Asp Tyr Leu Glu Gln Asp Leu Ala Met Thr Lys Asp Gly
Arg Leu Val Val Ile His Asp His Phe Leu Asp Gly Leu Thr Asp Val Ala Lys Lys Phe
Pro His Arg His Arg Lys Asp Gly Arg Tyr Tyr Val Ile Asp Phe Thr Leu Lys Glu Ile
Gln Ser Leu Glu Met Thr Glu Asn Phe Glu Thr Lys Asp Gly Lys Gln Ala Gln Val Tyr
Pro Asn Arg Phe Pro Leu Trp Lys Ser His Phe Arg Ile His Thr Phe Glu Asp Glu Ile
Glu Phe Ile Gln Gly Leu Glu Lys Ser Thr Gly Lys Lys Val Gly Ile Tyr Pro Glu Ile
Lys Ala Pro Trp Phe His His Gln Asn Gly Lys Asp Ile Ala Ala Glu Thr Leu Lys Val
Leu Lys Lys Tyr Gly Tyr Asp Lys Lys Thr Asp Met Val Tyr Leu Gln Thr Phe Asp Phe
Asn Glu Leu Lys Arg Ile Lys Thr Glu Leu Leu Pro Gln Met Gly Met Asp Leu Lys Leu
Val Gln Leu Ile Ala Tyr Thr Asp Trp Lys Glu Thr Gln Glu Lys Asp Pro Lys Gly Tyr
Trp Val Asn Tyr Asn Tyr Asp Trp Met Phe Lys Pro Gly Ala Met Ala Glu Val Val Lys
Tyr Ala Asp Gly Val Gly Pro Gly Trp Tyr Met Leu Val Asn Lys Glu Glu Ser Lys Pro
Asp Asn Ile Val Tyr Thr Pro Leu Val Lys Glu Leu Ala Gln Tyr Asn Val Glu Val His
Pro Tyr Thr Val Arg Lys Asp Ala Leu Pro Glu Phe Phe Thr Asp Val Asn Gln Met Tyr
Asp Ala Leu Leu Asn Lys Ser Gly Ala Thr Gly Val Phe Thr Asp Phe Pro Asp Thr Gly
Val Glu Phe Leu Lys Gly Ile Lys
SEQ ID NO: 140

| | |
|---|---|
| gcgaataccc aaatgaaatc agacaaaatc attattgctc accgtggtgc tagcggttat | 60 |
| ttaccagagc atacgttaga atctaaagca cttgcgtttg cacaacacgc agattattta | 120 |
| gagcaagatt tagcaatgac taaggatggt cgtttagtgg ttattcacga tcacttttta | 180 |
| gatggcttga ctgatgttgc gaaaaaattc ccacatcgtc accgtaaaga tggtcgttac | 240 |
| tatgtcatcg actttacctt aaaagaaatt caaagtttag aaatgactga aaactttgaa | 300 |
| accaaagacg gcaaacaagc gcaagtttat cctaatcgtt tcccactttg gaaatcacat | 360 |
| tttagaattc acacctttga agatgaaatt gagtttatcc aaggcttaga aaaatcgact | 420 |
| ggcagaaaag tagggattta tccagaaatc aaagcacctt ggttccacca tcaaaatggc | 480 |
| aaagatattg cagctgaaac gctcaaagtg ttaaaaaaat atggctatga taagaaaacc | 540 |
| gatatggttt acttacaaac tttcgatttt aatgaattaa aacgtatcaa aacggaatta | 600 |
| cttccacaaa tgggaatgga tttaaaatta gttcaattaa ttgcttatac agattggaaa | 660 |
| gaaacacaag aaaaagaccc aaagggttat tgggtaaact ataattacga ttggatgttt | 720 |
| aaacctggtg caatggcaga agtggttaaa tatgccgatg gtgttggccc aggttggtat | 780 |
| atgttagtta ataaagaaga atccaaacct gataatattg tgtacactcc gttggtaaaa | 840 |
| gaacttgcac aatataatgt ggaagtgcat ccttacaccg tgcgtaaaga tgcactgccc | 900 |
| gagtttttca cagacgtaaa tcaaatgtat gatgccttat tgaataaatc aggggcaaca | 960 |
| ggtgtattta ctgatttccc agatactgg | 989 |
| SEQ ID NO: 141 | |
| gcgaataccc aaatgaaatc agacaaaatc attattgctc accgtggtgc tagcggttat | 60 |
| ttaccagagc atacgttaga atctaaagca cttgcgtttg cacaacacgc agattattta | 120 |
| gagcaagatt tagcaatgac taaggatggt cgtttagtgg ttattcacga tcacttftta | 180 |
| gatggcttga ctgatgttgc gaaaaaattc ccacatcgtc accgtaaaga tggtcgttac | 240 |
| tatgtcatcg actttacctt aaaagaaatt caaagtttag aaatgactga aaactttgaa | 300 |
| accaaagacg gcaaacaagc gcaagtttat cctaatcgtt tcccactttg gaaatcacat | 360 |
| tttagaattc acacctttga agatgaaatt gagtttatcc aaggcttaga aaaatcgact | 420 |
| ggcagaaaag tagggattta tccagaaatc aaagcacctt ggttccacca tcaaaatggc | 480 |
| aaagatattg cagctgaaac gctcaaagtg ttaaaaaaat atggctatga taagaaaacc | 540 |
| gatatggttt acttacaaac tttcgatttt aatgaattaa aacgtatcaa aacggaatta | 600 |
| cttccacaaa tgggaatgga tttaaaatta gttcaattaa ttgcttatac agattggaaa | 660 |
| gaaacacaag aaaaagaccc aaagggttat tgggtaaact ataattacga ttggatgttt | 720 |
| aaacctggtg caatggcaga agtggttaaa tatgccgatg gtgttggccc aggttggtat | 780 |
| atgttagtta ataaagaaga atccaaacct gataatattg tgtacactcc gttggtaaaa | 840 |
| gaacttgcac aatataatgt ggaagtgcat ccttacaccg tgcgtaaaga tgcactgccc | 900 |
| gagtttttca cagacgtaaa tcaaatgtat gatgccttat tgaataaatc aggggcaaca | 960 |
| ggtgtattta ctgatttccc agatactgg | 989 |
| SEQ ID NO: 142 | |
| gcaaataccc aaatgaaatc tgacaaaatc atcattgctc atcgtggtgc tagcggttat | 60 |
| ttaccagagc atacgttaga atctaaagca cttgcgtttg cacagcacgc tgattactta | 120 |
| gagcaagatt tagcaatgac taaggatggt cgtttagtgg ttattcacga tcacttttta | 180 |
| gatggcttga ctgatgttgc gaaaaaattc ccacatcgtc accgtaaaga tggtcgttac | 240 |
| tatgtcatcg actttacctt aaaagaaatt caaagtttag aaatgacaga aaactttgaa | 300 |
| accaaagatg gcaaacagac acaagtttat cctaatcgtt tccccctttg gcaatcccat | 360 |
| ttccgtattc acacctttga agatgaaatt gaatttattc aaggtttaga aaaatcgacg | 420 |
| ggcaaaaaag tagggattta tccagaaatc aaagcacctt ggttccacca tcaaaatggc | 480 |
| aaagatattg ctgctgaaac gctcaaagtg ttaaaaaaat atggctatga taagaaaacc | 540 |
| gatatggttt acttacaaac tttcgatttt aatgaattaa aacgtatcaa aacggaatta | 600 |
| cttccacaaa tgggtatgga tttgaaatta gttcaattaa ttgcttatac agattggaaa | 660 |
| gaaacacaag aaaaagattc aaagggttat tgggtaaact ataattacga ttggatgttt | 720 |
| aaacctggtg caatggcaga agtggttaaa tatgccgatg gtgttggccc aggttggtat | 780 |
| atgttagtta ataaagaaga atccaaacct gataatattg tgtacactcc gttggtaaaa | 840 |
| gaacttgcac aatataatgt ggaagtgcat ccttacaccg tgcgtaaaga tgcactacct | 900 |
| gcgttifica cagacgtaaa tcaaatgtat gatgccttat tgaataaatc aggggcaaca | 960 |
| ggtgtattta ctgatttccc agatactgg | 989 |
| SEQ ID NO: 143 | |
| acctacggta ctaaataatt agcttaaaaa aggcggcggg caaattgctt agtcgccttt | 60 |
| tttgtaacta aaatctaaaa aaaaccataa aaatttaccg cactcttaag gagaaaatac | 120 |
| ttatgaaact taaaacttta gccctttctt tattagcagc tggcgtacta gcaggttgta | 180 |
| gcagccattc atcaaatatg gcgaataccc aaatgaaatc agacaaaatc attattgctc | 240 |
| accgtggtgc tagcggttat ttaccagagc atacgttaga atctaaagca cttgcgtttg | 300 |
| cacaacaggc tgattattta gagcaagatt tagcaatgac taaggatggt cgtttagtgg | 360 |
| ttattcacga tcacttttta gatggcttga ctgatgttgc gaaaaaattc ccacatcgtc | 420 |
| accgtaaaga tggccgttac tatgtcatcg actttacctt aaaagaaatt caaagtttag | 480 |
| aaatgacaga aaactttgaa accaaagatg gcaaacaagc gcaagtttat cctaatcgtt | 540 |
| tcccactttg gaaatcacat tttagaattc acacctttga agatgaaatt gaatttatcc | 600 |
| aaggcttaga aaaatccact ggcaaaaaag tagggattta tccagaaatc aaagcacctt | 660 |
| ggttccacca tcaaaatggt aaagatattg ctgctgaaac gctcaaagtg ttaaaaaaat | 720 |
| atggctatga taagaaaacc gatatggttt acttacaaac tttcgatttt aatgaattaa | 780 |
| aacgtatcaa aacggaatta cttccacaaa tgggtatgga tttgaaatta gttcaattaa | 840 |
| ttgcttatac agattggaaa gaaacacaag aaaaagatcc aaagggttat tgggtaaact | 900 |
| ataattacga ttggatgttt aaacctggag caatggcaga agtggttaaa tatgccgatg | 960 |
| gtgttggtcc aggttggtat atgttagtta ataaagaaga atccaaacct gataatattg | 1020 |
| tgtacactcc gttggtaaaa gaacttgcac aatataatgt ggaagtgcat ccttacaccg | 1080 |
| tgcgtaaaga tgcactaccc gcgtttttca cagatgtaaa tcaaatgtat gatgccttat | 1140 |
| tgaataaatc aggggcaaca ggtgtattta ctgatttccc agatactggc gtggaattct | 1200 |
| taaaaggaat aaaataatat ccctcacaac cgtgggtaaa catacccacg ttaactagg | 1259 |
| SEQ ID NO: 144 | |
| acttacggta ctaaataatt agcttaaaaa aggcggtggg taaattgctt agtcgccttt | 60 |
| tttgtaacta aaatctaaaa aaaccataaa aatttaccgc actcttaagg agaaaatact | 120 |
| tatgaaactt aaaactttag ccctttcttt attagcagct ggcgtactag caggttgtag | 180 |
| cagccattca tcaaatatgg cgaataccca aatgaaatca gacaaaatca ttattgctca | 240 |
| ccgtggtgct agcggttatt taccagagca tacgttagaa tctaaagcac ttgcgtttgc | 300 |
| acaacaggct gattatttag agcaagattt agcaatgact aaggatggtc gtttagtggt | 360 |
| tattcacgat cactttttag atggcttgac tgatgttgcg aaaaaattcc cacatcgtca | 420 |
| ccgtaaagat ggtcgttact atgtcatcga ctttacctta aaagaaattc aaagtttaga | 480 |
| aatgacagaa aactttgaaa ccaaagacgg caaacaagcg caagtttatc ctaatcgttt | 540 |
| cccactttgg aaatcacatt ttagaattca tacctttgaa gatgaaattg aatttatcca | 600 |
| aggcttagaa aaatccactg gcaaaaaagt agggatttat ccagaaatca aagcaccttg | 660 |
| gttccaccat caaaatggta aagatattgc tgctgaaacg ctcaaagtgt taaaaaaata | 720 |
| tggctatgat aagaaaaccg atatggttta cttacaaact ttcgatttta atgaattaaa | 780 |
| acgtatcaaa acggaattac ttccacaaat ggggatggat ttgaaattag ttcaattaat | 840 |
| tgcttataca gattggaaag aaacacaaga aaaagaccca aagggttatt gggtaaacta | 900 |
| taattacgat tggatgttta aacctggagc aatggcagaa gtggttaaat atgccgatgg | 960 |
| tgttggtcca ggttggtata tgttagttaa taaagaagaa tccaaacctg ataatattgt | 1020 |
| gtacactccg ttggtaaaag aacttgcaca atataatgtg gaagtgcatc cttacaccgt | 1080 |
| gcgtaaagat gcactgcccg agtttttcac agacgtaaat caaatgtatg atgtcttatt | 1140 |
| gaataaatca ggggcaacag gtgtatttac tgatttccca gatactggcg tggaattctt | 1200 |
| aaaaggaata aaataatatc cctcacaacc gtgggtaaac atacccacgt taactagg | 1258 |
| SEQ ID NO: 145 | |
| acttacggta ctaaataatt agcttaaaaa aggcggtggg caaattgctt agtcgccttt | 60 |
| tttgtaacta aaatctaaaa aaaccataaa aatttaccgc actttcaagg agaaaatact | 120 |
| tatgaaactt aaaactttag ccctttcttt attagcagct ggcgtactag caggttgtag | 180 |
| cagccattca tcaaatatgg cgaaaaccca aatgaaatca gacaaaatca ttattgctca | 240 |
| ccgtggtgct agcggttatt taccagagca tacgttagaa tctaaagcac ttgcgtttgc | 300 |
| acaacaggct gattatttag agcaagattt agcaatgact aaggatggtc gtttagtggt | 360 |
| tattcacgat cactttttag atggcttgac tgatgttgcg aaaaaattcc cacatcgtca | 420 |
| ccgtaaagat ggtcgttact atgtcatcga ctttacctta aaagaaattc aaagtttaga | 480 |
| aatgacagaa aactttgaaa ccaaagacgg caaacaagcg caagtttatc ctaatcgttt | 540 |
| ccccctttgg caatcccatt tccgtattca cacctttgaa gatgaaattg aatttatcca | 600 |
| aggcttagaa aaatcgactg gcagaaaagt agggatttat ccagaaatca aagcaccttg | 660 |
| gttccaccat caaaatggta aagatattgc tgctgaaacg ctcaaagtgt tgaaaaaata | 720 |
| tggctatgat aagaaaaccg atatggttta cttacaaact ttcgacttta atgaattaaa | 780 |
| acgtatcaaa acggaattac ttccacaaat gggtatggat ttgaaattag ttcaattaat | 840 |
| tgcttataca gattggaaag aaacacaaga aaaagattca aagggttatt gggtaaacta | 900 |
| taattacgat tggatgttta aacctggtgc aatggcagaa gtggttaaat atgccgatgg | 960 |
| tgttggccca ggttggtata tgttagttaa taaagaagaa tccaaacctg ataatattgt | 1020 |
| gtacactccg ttggtaaaag aacttgcaaa atataatgtg gaagtgcatc cttacaccgt | 1080 |
| gcgtaaagat gcactgcctg cgtttttcac agacgtaaat caaatgtatg atgctttatt | 1140 |
| gaataaatca ggggcaacag gtgtatttac tgatttccca gatactggcg tggaattctt | 1200 |
| aaaaggaata gaataatatc cctcacaacc gtgggtaaac atacccacgg tt | 1252 |
| SEQ ID NO: 146 | |
| gatcggcggt ggcgtattag cggtgttatt actcttaatc gtaatggttg aagaaggaaa | 60 |
| acacaaagcg aaattaggcg atacttacgg tactaaataa ttagcttaaa aaaggcggtg | 120 |
| ggcaaattgc ttagtcgcct tttttgtaac taaaatctaa aaactctata aaaatttacc | 180 |
| gcactcttaa ggagaaaata cttatgaaac ttaaaacttt agccctttct ttattagcag | 240 |
| ctggcgtact agcaggttgt agcagccatt catcaaatat ggcgaatacc caaatgaaat | 300 |
| cagacaaaat cattattgct caccgtggtg ctagcggtta tttaccagag catacgttag | 360 |
| aatctaaagc acttgcgttt gcacaacagg ctgattattt agagcaagat ttagcaatga | 420 |
| ctaaggatgg tcgtttagtg gttattcacg atcacttttt agatggcttg actgatgttg | 480 |
| cgaaaaaatt cccacatcgt catcgtaaag atggccgtta ctatgtcatc gactttacct | 540 |
| taaaagaaat tcaaagttta gaaatgacag aaaactttga aaccaaagat ggcaaacaag | 600 |
| cgcaagttta tcctaatcgt ttccctcttt ggaaatcaca ttttagaatt catacctttg | 660 |
| aagatgaaat tgaatttatc caaggcttag aaaaatccac tggcaaaaaa gtagggattt | 720 |
| atccagaaat caaagcacct tggttccacc atcaaaatgg taaagatatt gctgctgaaa | 780 |
| cgctcaaagt gttaaaaaaa tatggctatg ataagaaaac cgatatggtt tacttacaaa | 840 |
| ctttcgattt taatgaatta aaacgtatca aaacggaatt acttccacaa atgggaatgg | 900 |
| atttgaaatt agttcaatta attgcttata cagattggaa agaaacacaa gaaaaagacc | 960 |
| caaagggtta ttgggtaaac tataattacg attggatgtt taaacctggt gcaatggcag | 1020 |
| aagtggttaa atatgccgat ggtgttggcc caggttggta tatgttagtt aataaagaag | 1080 |
| aatccaaacc tgataatatt gtgtacactc cgttggtaaa agaacttgca caatataatg | 1140 |
| tggaagtgca tccttacacc gtgcgtaaag atgcactgcc cgagifittc acagacgtaa | 1200 |
| atcaaatgta tgatgcctta ttgaataaat caggggcaac aggtgtattt actgatttcc | 1260 |
| cagatactgg cgtggaattc ttaaaaggaa taaaataata tccctcacaa ccgtgggtaa | 1320 |
| acatacccac ggttaactag gtttctatat cgtagaaact aaaaatctac tctaacagag | 1380 |
| taacatcata atcaatctag gtgttctaac ctagaattca aataaggagg ctatttcaaa | 1440 |
| acactccgta ttcttffita ataaattctc ttccctttac ttagggaaaa cactcttcat | 1500 |
| ttcaaccgca cttctaagga gtgctctatg gataaatcat taaaagcgaa ctgtattggc | 1560 |
| gagtttttag gtacagcctt attgattttc tttggtgtgg gctgcgttgc agcactaaaa | 1620 |
| gtagcaggcg ctagttttgg cttgtgggaa atcagcatta tgtgggggat gggcgttgca | 1680 |
| cttgcagtat atgcaacagc gggtttatct ggcgcacatt taaaccctgc agtaaccatt | 1740 |
| gccctttgga aatttgcttg ctttgatggc aaaaaagtaa ttccttacat catttcacaa | 1800 |
| atgctcggcg cattctttgc tgccgcatta gtttatgcct tataccgcaa tgtttttatc | 1860 |
| gatc | 1864 |

SEQ ID NO: 147
SSHSSNMANT
SEQ ID NO: 148 - UspA2 ATCC 25238
MKTMKLLPLKIAVTSAMIIGLGAASTANAQAKNDITLEDLPYLIKKIDQNELEADIGDITALEKYLALSQYGN
ILALEELNKALEELDEDVGWNQNDIANLEDDVETLTKNQNALAEQGEAIKEDLQGLADFVEGQEGKILQNETS
IKKNTQRNLVNGFEIEKNKDAIAKNNESIEDLYDFGHEVAESIGEIHAHNEAQNETLKGLIINSIENTNNITK
NKADIQALENNVVEELFNLSGRLIDQKADIDNNINNIYELAQQQDQHSSDIKTLKKNVEEGLLELSGHLIDQK
TDIAQNQANIQDLATYNELQDQYAQKQTEAIDALNKASSENTQNIEDLAAYNELQDAYAKQQTEAIDALNKAS
SENTQNIEDLAAYNELQDAYAKQQTEAIDALNKASSENTQNIAKNQADIANNINNIYELAQQQDQHSSDIKTL
AKASAANTDRIAKNKADADASFETLTKNQNTLIEKDKEHDKLITANKTAIDANKASADTKFAATADAITKNGN
AITKNAKSITDLGTKVDGFDSRVTALDTKVNAFDGRITALDSKVENGMAAQAALSGLFQPYSVGKFNATAALG
GYGSKSAVAIGAGYRVNPNLAFKAGAAINTSGNKKGSYNIGVNYEF
SEQ ID NO: 149
MC-001 (protein) - (M)(UspA2 amino acids 30-540)(ASHHHHHH)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAIAK
NNESIEDLYDFGHEVAESIGEIHAHNEAQNETLKGLIINSIENTNNITKNKADIQALENNVVEELFNLSGR
LIDQKADIDNNINNIYELAQQQDQHSSDIKTLKKNVEEGLLELSGHLIDQKTDIAQNQANIQDLATYNELQ
DQYAQKQTEAIDALNKASSENTQNIEDLAAYNELQDAYAKQQTEAIDALNKASSENTQNIEDLAAYNELQD
AYAKQQTEAIDALNKASSENTQNIAKNQADIANNINNIYELAQQQDQHSSDIKTLAKASAANTDRIAKNKA
DADASFETLIKNQNTLIEKDKEHDKLITANKTAIDANKASADTKFAATADAITKNGNAITKNAKSITDLGT
KVDGFDSRVTALDTKASHHHHHH
SEQ ID NO: 150
MC-002 (Protein) - (M)(UspA2 amino acids 30-540)
MQAKNDITLEDLPYLIKKIDQNELEADIGDITALEKYLALSQYGNILALEELNKALEELDEDVGWNQNDIA
NLEDDVETLIKNQNALAEQGEAIKEDLQGLADEVEGQEGKILQNETSIKKNIQRNLVNGFEIEKNKDAIAK
NNESIEDLYDFGHEVAESIGEIHAHNEAQNETLKGLITNSIENTNNITKNKADIQALENNVVEELFNLSGR
LIDQKADIDNNINNIYELAQQQDQHSSDIKTLKKNVEEGLLELSGHLIDQKTDIAQNQANIQDLATYNELQ
DQYAQKQTEAIDALNKASSENTQNIEDLAAYNELQDAYAKQQTEAIDALNKASSENTQNIEDLAAYNELQD
AYAKQQTEAIDALNKASSENTQNIAKNQADIANNINNIYELAQQQDQHSSDIKTLAKASAANTDRIAKNKA
DADASFETLIKNQNTLIEKDKEHDKLITANKTAIDANKASADTKFAATADAITKNGNAITKNAKSITDLGT
KVDGFDSRVTALDTK
SEQ ID NO: 151
MC-003 (Protein) - (M)(UspA2 amino acids 30-540)(H)
MQAKNDITLEDLPYLIKKIDQNELEADIGDITALEKYLALSQYGNILALEELNKALEELDEDVGWNQNDIA
NLEDDVETLIKNQNALAEQGEAIKEDLQGLADEVEGQEGKILQNETSIKKNIQRNLVNGFEIEKNKDAIAK
NNESIEDLYDFGHEVAESIGEIHAHNEAQNETLKGLITNSIENTNNITKNKADIQALENNVVEELFNLSGR
LIDQKADIDNNINNIYELAQQQDQHSSDIKTLKKNVEEGLLELSGHLIDQKTDIAQNQANIQDLATYNELQ
DQYAQKQTEAIDALNKASSENTQNIEDLAAYNELQDAYAKQQTEAIDALNKASSENTQNIEDLAAYNELQD
AYAKQQTEAIDALNKASSENTQNIAKNQADIANNINNIYELAQQQDQHSSDIKTLAKASAANTDRIAKNKA
DADASFETLIKNQNTLIEKDKEHDKLITANKTAIDANKASADTKFAATADAITKNGNAITKNAKSITDLGT
KVDGFDSRVTALDTKH
SEQ ID NO: 152
MC-004 (Protein) - (M)(UspA2 amino acids 30-540)(HH)
MQAKNDITLEDLPYLIKKIDQNELEADIGDITALEKYLALSQYGNILALEELNKALEELDEDVGWNQNDIA
NLEDDVETLIKNQNALAEQGEAIKEDLQGLADEVEGQEGKILQNETSIKKNIQRNLVNGFEIEKNKDAIAK
NNESIEDLYDFGHEVAESIGEIHAHNEAQNETLKGLITNSIENTNNITKNKADIQALENNVVEELFNLSGR
LIDQKADIDNNINNIYELAQQQDQHSSDIKTLKKNVEEGLLELSGHLIDQKTDIAQNQANIQDLATYNELQ
DQYAQKQTEAIDALNKASSENTQNIEDLAAYNELQDAYAKQQTEAIDALNKASSENTQNIEDLAAYNELQD
AYAKQQTEAIDALNKASSENTQNIAKNQADIANNINNIYELAQQQDQHSSDIKTLAKASAANTDRIAKNKA
DADASFETLIKNQNTLIEKDKEHDKLITANKTAIDANKASADTKFAATADAITKNGNAITKNAKSITDLGT
KVDGFDSRVTALDTKHH
SEQ ID NO: 153
MC-005 (Protein) - (M)(UspA2 amino acids 30-519)(ASHHHHHH)
MQAKNDITLEDLPYLIKKIDQNELEADIGDITALEKYLALSQYGNILALEELNKALEELDEDVGWNQNDIA
NLEDDVETLIKNQNALAEQGEAIKEDLQGLADEVEGQEGKILQNETSIKKNIQRNLVNGFEIEKNKDAIAK
NNESIEDLYDFGHEVAESIGEIHAHNEAQNETLKGLITNSIENTNNITKNKADIQALENNVVEELFNLSGR
LIDQKADIDNNINNIYELAQQQDQHSSDIKTLKKNVEEGLLELSGHLIDQKTDIAQNQANIQDLATYNELQ
DQYAQKQTEAIDALNKASSENTQNIEDLAAYNELQDAYAKQQTEAIDALNKASSENTQNIEDLAAYNELQD
AYAKQQTEAIDALNKASSENTQNIAKNQADIANNINNIYELAQQQDQHSSDIKTLAKASAANTDRIAKNKA
DADASFETLIKNQNTLIEKDKEHDKLITANKTAIDANKASADTKFAATADAITKNGNAITKNAKSASHHHH
HH
SEQ ID NO: 154
MC-006 (Protein) - (M)(UspA2 amino acids 30-519)
MQAKNDITLEDLPYLIKKIDQNELEADIGDITALEKYLALSQYGNILALEELNKALEELDEDVGWNQNDIA
NLEDDVETLIKNQNALAEQGEAIKEDLQGLADEVEGQEGKILQNETSIKKNIQRNLVNGFEIEKNKDAIAK
NNESIEDLYDFGHEVAESIGEIHAHNEAQNETLKGLITNSIENTNNITKNKADIQALENNVVEELFNLSGR
LIDQKADIDNNINNIYELAQQQDQHSSDIKTLKKNVEEGLLELSGHLIDQKTDIAQNQANIQDLATYNELQ
DQYAQKQTEAIDALNKASSENTQNIEDLAAYNELQDAYAKQQTEAIDALNKASSENTQNIEDLAAYNELQD
AYAKQQTEAIDALNKASSENTQNIAKNQADIANNINNIYELAQQQDQHSSDIKTLAKASAANTDRIAKNKA
DADASFETLIKNQNTLIEKDKEHDKLITANKTAIDANKASADTKFAATADAITKNGNAITKNAKS
SEQ ID NO: 155
MC-007 (Protein) - (M)(UspA2 amino acids 30-564)(ASHHHHHH)
MQAKNDITLEDLPYLIKKIDQNELEADIGDITALEKYLALSQYGNILALEELNKALEELDEDVGWNQNDIA
NLEDDVETLIKNQNALAEQGEAIKEDLQGLADEVEGQEGKILQNETSIKKNIQRNLVNGFEIEKNKDAIAK
NNESIEDLYDFGHEVAESIGEIHAHNEAQNETLKGLITNSIENTNNITKNKADIQALENNVVEELFNLSGR
LIDQKADIDNNINNIYELAQQQDQHSSDIKTLKKNVEEGLLELSGHLIDQKTDIAQNQANIQDLATYNELQ
DQYAQKQTEAIDALNKASSENTQNIEDLAAYNELQDAYAKQQTEAIDALNKASSENTQNIEDLAAYNELQD
AYAKQQTEAIDALNKASSENTQNIAKNQADIANNINNIYELAQQQDQHSSDIKTLAKASAANTDRIAKNKA
DADASFETLIKNQNTLIEKDKEHDKLITANKTAIDANKASADTKFAATADAITKNGNAITKNAKSITDLGT
KVDGFDSRVTALDTKVNAFDGRITALDSKVENGMAAQAAASHHHHHH
SEQ ID NO: 156
MC-008 (Protein) - (M)(UspA2 30-564)(HH)
MQAKNDITLEDLPYLIKKIDQNELEADIGDITALEKYLALSQYGNILALEELNKALEELDEDVGWNQNDIA
NLEDDVETLIKNQNALAEQGEAIKEDLQGLADEVEGQEGKILQNETSIKKNIQRNLVNGFEIEKNKDAIAK
NNESIEDLYDFGHEVAESIGEIHAHNEAQNETLKGLITNSIENTNNITKNKADIQALENNVVEELFNLSGR
LIDQKADIDNNINNIYELAQQQDQHSSDIKTLKKNVEEGLLELSGHLIDQKTDIAQNQANIQDLATYNELQ
DQYAQKQTEAIDALNKASSENTQNIEDLAAYNELQDAYAKQQTEAIDALNKASSENTQNIEDLAAYNELQD
AYAKQQTEAIDALNKASSENTQNIAKNQADIANNINNIYELAQQQDQHSSDIKTLAKASAANTDRIAKNKA
DADASFETLIKNQNTLIEKDKEHDKLITANKTAIDANKASADTKFAATADAITKNGNAITKNAKSITDLGT
KVDGFDSRVTALDTKVNAFDGRITALDSKVENGMAAQAAHH
SEQ ID NO: 157
MC-009 (Protein) - (M)(UspA2 31-564)(HH)
MAKNDITLEDLPYLIKKIDQNELEADIGDITALEKYLALSQYGNILALEELNKALEELDEDVGWNQNDIAN
LEDDVETLTKNQNALAEQGEAIKEDLQGLADEVEGQEGKILQNETSIKKNTQRNLVNGFEIEKNKDATAKN
NESIEDLYDFGHEVAESIGEIHAHNEAQNETLKGLITNSIENTNNITKNKADIQALENNVVEELFNLSGRL
IDQKADIDNNINNIYELAQQQDQHSSDIKTLKKNVEEGLLELSGHLIDQKTDIAQNQANIQDLATYNELQD
QYAQKQTEAIDALNKASSENTQNIEDLAAYNELQDAYAKQQTEAIDALNKASSENTQNIEDLAAYNELQDA
YAKQQTEAIDALNKASSENTQNIAKNQADIANNINNIYELAQQQDQHSSDIKTLAKASAANTDRIAKNKAD
ADASFETLTKNQNTLIEKDKEHDKLITANKTAIDANKASADTKFAATADAITKNGNAITKNAKSITDLGTK
VDGFDSRVTALDTKVNAFDGRITALDSKVENGMAAQAAHH
SEQ ID NO: 158
MC-010 (Protein) - (M)(UspA2 amino acids 30-564)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADEVEGQEGKILQNETSIKKNTQRNLVNGFEIEKNKDATAK
NNESIEDLYDFGHEVAESIGEIHAHNEAQNETLKGLITNSIENTNNITKNKADIQALENNVVEELFNLSGR
LIDQKADIDNNINNIYELAQQQDQHSSDIKTLKKNVEEGLLELSGHLIDQKTDIAQNQANIQDLATYNELQ
DQYAQKQTEAIDALNKASSENTQNIEDLAAYNELQDAYAKQQTEAIDALNKASSENTQNIEDLAAYNELQD
AYAKQQTEAIDALNKASSENTQNIAKNQADIANNINNIYELAQQQDQHSSDIKTLAKASAANTDRIAKNKA
DADASFETLTKNQNTLIEKDKEHDKLITANKTAIDANKASADTKFAATADAITKNGNAITKNAKSITDLGT
KVDGFDSRVTALDTKVNAFDGRITALDSKVENGMAAQAA
SEQ ID NO: 159
MC-011 (Protein) - (M)(UspA2 amino acids 31-540)(ASHHHHHH)
MAKNDITLEDLPYLIKKIDQNELEADIGDITALEKYLALSQYGNILALEELNKALEELDEDVGWNQNDIAN
LEDDVETLTKNQNALAEQGEAIKEDLQGLADEVEGQEGKILQNETSIKKNTQRNLVNGFEIEKNKDATAKN
NESIEDLYDFGHEVAESIGEIHAHNEAQNETLKGLITNSIENTNNITKNKADIQALENNVVEELFNLSGRL
IDQKADIDNNINNIYELAQQQDQHSSDIKTLKKNVEEGLLELSGHLIDQKTDIAQNQANIQDLATYNELQD
QYAQKQTEAIDALNKASSENTQNIEDLAAYNELQDAYAKQQTEAIDALNKASSENTQNIEDLAAYNELQDA
YAKQQTEAIDALNKASSENTQNIAKNQADIANNINNIYELAQQQDQHSSDIKTLAKASAANTDRIAKNKAD
ADASFETLTKNQNTLIEKDKEHDKLITANKTAIDANKASADTKFAATADAITKNGNAITKNAKSITDLGTK
VDGFDSRVTALDTKASHHHHHH
SEQ ID NO: 160: PilA mAb VH Region (DNA)
CAGGGTCAGCTGCAGCAGTCTGGACCTGAGCTGGTGAAGCCTGGGGCTTCAGTGAAGATGTCCTGCAAGGCTT
CTGGATACACATTCACTGACTATGTTATAAGCTGGGTGAAGCAGAGAATTGGACAGGGCCTTGAGTGGATTGG
AGAGATTCATCCTGGAAGTGGTAGTATTCACTACAATGAGAAGTTCAAGGGCAAGGCCACACTGACTGCAGAC
AAATCCTCCAACACAGCCTACATGCAGCTCAGCAGCCTGACATCTGAGGACTCTGCGGTCTATTTCTGTGCAA
GAAGGGGGTTACGACGTCCCTGGTTTGCTTACTGGGGCCAAGGGACTCTGGTCACTGTCTCTGCAG
SEQ ID NO: 161 - PilA mAb VH Region (Protein)
QGQLQQSGPELVKPGASVKMSCKASGYTFTDYVISWVKQRIGQGLEWIGEIHPGSGSIHYNEKFKGKATLTAD
KSSNTAYMQLSSLTSEDSAVYFCARRGLRRPWFAYWGQGTLVTVSA
SEQ ID NO: 162 - PilA mAb VL Region (DNA)
GACATTGTGCTGACACAGTCTCCTGCTTCCTTAGCTGTATCTCTGGGGCAGAGGGCCACCATCTCATGCAGGG
CCAGCAAAAGTGTCAGTACATCTGGCTATAGTTATATGCACTGGTTCCAACAGAAACCAGGACAGCCACCCAA
ACTCCTCATCTATCTTGCATCCAACCTAGAATCTGGGGTCCCTGCCAGGTTCAGTGGCAGTGGGTCTGGGACA
GACTTCACCCTCAACATCCATCCTGTGGAGGAGGAGGATGCTGCAACCTATTACTGTCAGCACAGTAGGGAGC
TTCCGCTCACGTTCGGTGCTGGGACCAAGCTGGAGCTGAAA
SEQ ID NO: 163 - PilA mAb VL Region (Protein)
DIVLTQSPASLAVSLGQRATISCRASKSVSTSGYSYMHWFQQKPGQPPKLLIYLASNLESGVPARFSGSGSGT
DFTLNIHPVEEEDAATYYCQHSRELPLTFGAGTKLELK
SEQ ID NO: 164 - CDR-H1
DYVIS
SEQ ID NO: 165 - CDR-H2
EIHPGSGSIHYNEKFKG
SEQ ID NO: 166 - CDR-H3
RGLRRPWFAY
SEQ ID NO: 167 - CDR-L1
RASKSVSTSGYSYMH
SEQ ID NO: 168 - CDR-L2
LASNLES
SEQ ID NO: 169 - CDR-L3
QHSRELPLT

1-16. (canceled)

17. An antigen binding protein which binds to Protein E at one or more of amino acid residues within I41 to Y49 (e.g. SEQ ID NO: 133) and Y141 to A154 (e.g. SEQ ID NO: 134) of Protein E, said antigen binding protein comprising a VH region comprising a sequence at least 80% identical to the sequence of SEQ ID NO: 124; and/or a VL region comprising a sequence at least 80% identical to the sequence of SEQ ID NO: 129.

18. The antigen binding protein according to claim 17 which binds to Protein E in its native conformation with a higher specificity and/or affinity than to Protein E in a non-native conformation.

19. The antigen binding protein according to claim 17 which inhibits laminin binding.

20. An antigen binding protein comprising:
(i) any one or a combination of CDRs selected from CDR-H1 (SEQ ID NO: 125), CDR-H2 (SEQ ID NO: 126) or CDR-H3 (SEQ ID NO: 127), and/or CDR-L1 (SEQ ID NO: 130), CDR-L2 (SEQ ID NO: 131) or CDR-L3 (SEQ ID NO: 132) or
(ii) a CDR variant of (i), wherein the variant has 1, 2, or 3 amino acid modifications in each CDR, which is able to bind to Protein E at one or more of amino acid residues within I41 to Y49 and Y141 to A154 of Protein E (e.g. SEQ ID NO: 133 and SEQ ID NO: 134).

21. The antigen binding protein of claim 20, comprising CDR-H1 (SEQ ID NO: 125), CDR-H2 (SEQ ID NO: 126) and CDR-H3 (SEQ ID NO: 127) and/or CDR-L1 (SEQ ID NO: 130), CDR-L2 (SEQ ID NO: 131) and CDR-L3 (SEQ ID NO: 132).

22. The antigen binding protein of claim 20, comprising the VH as set forth in SEQ ID NO:123 and the VL as set forth in SEQ ID NO:124.

23. The antigen binding protein of claim 20, wherein the antigen binding protein is an isolated monoclonal antibody or fragment thereof that binds to Protein E and comprises CDR-H1 (SEQ ID NO: 125), CDR-H2 (SEQ ID NO: 126) and CDR-H3 (SEQ ID NO: 127) and CDR-L1 (SEQ ID NO: 130), CDR-L2 (SEQ ID NO: 131) and CDR-L3 (SEQ ID NO: 132).

24. An assay comprising exposing a sample of a test antigen to an antigen binding protein according to claim 20 and measuring the amount of antigen binding protein bound to the test antigen.

25. The assay of claim 24 wherein the assay is an in vitro assay.

26. The assay of claim 25 wherein the assay is an ELISA or a sandwich ELISA.

27. The assay of claim 24 further comprising comparing the amount of antigen binding protein bound to the test antigen to the amount of antigen binding protein bound to a reference sample.

28. The assay of claim 24 wherein the assay is to determine or measure the presence of a test antigen in its native conformation.

29. The assay of claim 24, wherein the assay detects or measures the change in the conformation of Protein E as compared to its native conformation.

30. The assay of claim 24 wherein the assay determines or measures the potency of a test antigen.

31. The assay of claim 24 wherein the test antigen comprises Protein E and/or PilA.

32. The assay of claim 24 wherein the test antigen comprises a fusion protein of Protein E and PilA.

33. The assay of claim 24 wherein the test antigen is LVL-735 (SEQ ID NO: 122) or sequences with at least 80% identity to LVL-735 (SEQ ID NO: 122).

34. A method for in vitro analysis of a test antigen, comprising steps of: (i) performing the assay of claim 24 on a test antigen and a reference sample of known potency; and (ii) comparing the results from step (i) to determine the potency of the test antigen relative to the reference sample.

35. A method for analysing a batch of vaccine, comprising steps of: (i) assaying a test antigen taken from a batch of vaccine by the method of claim 34 and, if the results of step (i) indicate an acceptable relative potency, (ii) releasing vaccine from the batch for in vivo use.

36. The antigen binding protein according to claim 20 which inhibits laminin binding.
